(19) United States
(12) Patent Application Publication
(10) Pub. No.: US 2010/0196347 A1
(43) Pub. Date: Aug. 5, 2010

(54) BONE GRAFTS WITH REDUCED PROTEASE ACTIVITY AND METHODS OF SELECTION AND USE

(76) Inventors: Vladimir Kery, Franklin, TN (US); Conan S. Young, Franklin, TN (US); Leo B. Snel, Franklin, TN (US); Charles E. Hart, Brentwood, TN (US)

Correspondence Address: Biomimetic Therapeutics Inc.;C/O MoFo Palo Alto, 755 Page Mill Road, Palo Alto, CA, 94304-1018, US

(21) Appl. No.: 12/642,677

(22) Filed: Dec. 18, 2009

Related U.S. Application Data
(60) Provisional application No. 61/139,448, filed on Dec. 19, 2008.
(60) Provisional application No. 61/149,998, filed on Feb. 4, 2009.
(60) Provisional application No. 61/154,311, filed on Feb. 20, 2009.

(51) Int. Cl. A61K 38/48; C12Q 1/37; C12N 9/99; A61K 35/32

(52) U.S. Cl. 424/94.63; 435/23; 435/184; 424/549

(57) ABSTRACT

The invention features bone grafts (such as bone allografts) with reduced protease activity. These bone grafts are useful, for example, in conjunction with a polypeptide of interest (such as platelet derived growth factor) for treating, stabilizing, preventing, and/or delaying a bone, periodontium, ligament, cartilage, or tendon condition in an individual (such as a human). Additionally, the invention provides methods of measuring the protease activity associated with a bone graft, reducing the level of protease activity associated with a bone graft, selecting a bone graft with an acceptable level of protease activity, and methods of administering a bone graft and a polypeptide of interest to an individual.

### CROSS-REFERENCE TO RELATED APPLICATIONS

This application claims the benefit under 35 U.S.C. §119(e) of U.S. Provisional Application Ser. Nos. 61/139,448, filed Dec. 19, 2008, 61/149,998, filed Feb. 4, 2009, and 61/154,311 filed Feb. 20, 2009, the contents of which are incorporated herein by reference in their entirety.

### BACKGROUND OF THE INVENTION

Bone grafts (such as bone allografts) can be transplanted into an individual to facilitate the healing of bone, to strengthen bone, and/or to improve bone function. In some cases, bone from a human donor is transplanted into another human. Exemplary human bone allografts are pieces of bone skeleton isolated post mortem from human donors.

One or more polypeptides (such as growth factors) may be administered in conjunction with the bone graft to increase the effectiveness of the bone graft. For example, recombinant human platelet-derived growth factor BB (rhPDGF-BB) is a potent wound healing polypeptide and stimulator of the proliferation and recruitment of bone cells. In particular, a combination of human bone graft and PDGF can be used for bone regeneration in bone healing of fractures and other bone injuries (see, for example, U.S. App. Pub. No. 2007/0207185, filed Feb. 9, 2007).

Improved bone grafts (such as bone allografts) are needed for administration in conjunction with a polypeptide of interest (such as a polypeptide that promotes bone repair, healing, or growth) for treating, stabilizing, preventing, and/or delaying a bone, periodontium, ligament, cartilage, or tendon condition, disease, or defect. Desirably, the bone graft has minimal effect on the biological function and/or structure of the polypeptide of interest.

### BRIEF SUMMARY OF THE INVENTION

In one aspect, the invention features methods of selecting a bone graft (such as a bone allograft) for administration to an individual in conjunction with a polypeptide of interest (e.g., platelet derived growth factor (PDGF)). In some embodiments, the method involves measuring the protease activity associated with a bone graft, whereby the amount of protease activity associated with the bone graft determines whether the bone graft is selected for administration to the individual in conjunction with the polypeptide of interest. In some embodiments, the method involves selecting a bone graft with an acceptable level of protease activity for administration to the individual in conjunction with the polypeptide of interest. In some embodiments, the method of selecting a bone graft for administration to an individual in conjunction with PDGF includes selecting a bone graft with a protease activity of less than about 50 trypsin equivalents (wherein 1 trypsin equivalent is the amount of protease activity equivalent to 1 ng of trypsin using a protease substrate, e.g. succinylated casein in a QuantiCleave protease assay kit (Pierce, Rockford, Ill.)) for administration to the individual in conjunction with PDGF. In some embodiments, the method of selecting a bone graft for administration to an individual in conjunction with PDGF includes selecting a bone graft with a protease activity of between about 50 to about 65 trypsin equivalents (such as about 50 to about 55, about 55 to about 60, or about 60 to about 65 trypsin equivalents) for administration to the individual in conjunction with PDGF. In some embodiments, the method of selecting a bone graft for administration to an individual in conjunction with PDGF includes selecting a bone graft with a protease activity of less than about 50 trypsin equivalents (such as less than about 45, less that about 40, less than about 35, less that about 30, less than about 25, less than about 20, less than about 15, less than about 10, less than about 5, about 0 trypsin equivalents) for administration to the individual in conjunction with PDGF. In some embodiments, the method of selecting a bone graft for administration to an individual in conjunction with PDGF includes selecting a bone graft with a protease activity of about any of 50, 55, 60, or 65 trypsin equivalents for administration to the individual in conjunction with PDGF. In some embodiments of any of the methods, the method includes administering the selected bone graft and the polypeptide of interest to the individual. In some embodiments, the protease activity of two or more bone grafts is measured, and the bone graft with the lowest protease activity is administered to the individual in conjunction with the polypeptide of interest. In some embodiments, the protease activity of the selected bone graft is less than about 50 trypsin equivalents. In some embodiments, the protease activity of the selected bone graft is between about 50 to about 65 trypsin equivalents (such as about 50 to about 55, about 55 to about 60, or about 60 to about 65 trypsin equivalents). In some embodiments, the protease activity of the selected bone graft is about any of 50, 55, 60, or 65 trypsin equivalents. In some embodiments, the protease activity of the selected bone graft is less than about 50 trypsin equivalents (such as less than about 45, less that about 40, less than about 35, less that about 30, less than about 25, less than about 20, less than about 15, less than about 10, less than about 5, about 0 trypsin equivalents).

In some embodiments of any of the methods, measuring the protease activity associated with a bone graft comprises (a) removing at least a portion of the total amount of a protease associated with the bone graft from the bone graft; and (b) measuring the amount of a polypeptide substrate that is cleaved by the removed protease, thereby determining the amount of protease activity associated with the bone graft. In some embodiments, step (a) comprises increasing the ionic strength of the solution comprising the bone graft and protease. In some embodiments, step (a) comprises incubating the bone graft and protease in a salt solution. In some embodiments, the salt solution is a NaCl solution. In some embodiments, the salt solution contains between about 0.15 M NaCl and about 1.5 M NaCl or between about 0.3 M NaCl and about 1.5 M NaCl. In some embodiments, the salt solution contains about 0.3 M NaCl. In some embodiments, step (b) comprises separating the cleaved polypeptide substrate, the uncleaved polypeptide substrate, and the bone graft. In some embodiments, high-performance liquid chromatography is used to separate the cleaved polypeptide substrate, the uncleaved polypeptide substrate, and the bone graft. In some embodiments, size exclusion chromatography is used to separate the cleaved polypeptide substrate, the uncleaved polypeptide substrate, and the bone graft. In some embodiments, another separation method is used to separate the cleaved polypeptide substrate, the uncleaved polypeptide substrate, and/or the bone graft. Other exemplary separation methods include simple centrifugation, ion exchange chromatography, and electrophoresis.

In some embodiments of any of the methods, measuring the protease activity associated with a bone graft comprises measuring the amount of a polypeptide substrate that is cleaved by a protease activity associated with the bone graft. In some embodiments, measuring the amount of cleaved polypeptide substrate comprises (a) incubating the polypeptide substrate with the bone graft, (b) removing at least a portion of the total amount of cleaved polypeptide substrate from the bone graft, and (c) measuring the amount of cleaved polypeptide substrate. In some embodiments, step (b) comprises increasing the ionic strength of the solution comprising the bone graft and the polypeptide substrate. In some embodiments, step (b) comprises incubating the bone graft and the polypeptide substrate in a salt solution. In some embodiments, the salt solution is a NaCl solution. In some embodiments, the salt solution contains between about 0.15 M and about 2.0 M NaCl. In some embodiments, the salt solution contains about 0.6 M NaCl. In some embodiments, step (c) comprises separating the cleaved polypeptide substrate, the uncleaved polypeptide substrate, and the bone graft. In some embodiments, high-performance liquid chromatography is used to separate the cleaved polypeptide substrate, the uncleaved polypeptide substrate, and the bone graft. In some embodiments, size exclusion chromatography is used to separate the cleaved polypeptide substrate, the uncleaved polypeptide substrate, and the bone graft. In some embodiments, another separation method is used to separate the cleaved polypeptide substrate, the uncleaved polypeptide substrate, and/or the bone graft. Other exemplary separation methods include simple centrifugation, ion exchange chromatography, and electrophoresis.

In some embodiments of any of the methods, the polypeptide of interest and the polypeptide substrate are the same. In some embodiments, the polypeptide of interest and the polypeptide substrate are different. In some embodiments, the polypeptide of interest is PDGF. In some embodiments, the bone graft includes calcium phosphate (such as β-tricalcium phosphate) that has been added to the bone graft. In some embodiments, the bone graft includes one or more other compounds (such as glycerin) that have been added to the bone graft.

In one aspect, the invention features methods for measuring the protease activity associated with a bone graft (such as a bone allograft). In some embodiments, the method includes (a) removing at least a portion of the total amount of a protease associated with the bone graft from the bone graft; and (b) measuring the amount of a polypeptide substrate that is cleaved by the removed protease, thereby determining the amount of protease activity associated with the bone graft. In some embodiments, the method for measuring the protease activity associated with a bone graft includes (a) removing at least a portion of the total amount of a protease associated with the bone graft from the bone graft by incubating the bone graft in a salt solution containing about 0.15 M NaCl to about 1.5 M NaCl (such as between about 0.3 M NaCl and about 1.5 M NaCl); and (b) measuring the amount of PDGF that is cleaved by the removed protease, thereby determining the amount of protease activity associated with the bone graft. In some embodiments of any of the methods, step (a) comprises increasing the ionic strength of the solution comprising the bone graft and protease. In some embodiments, step (a) comprises incubating the bone graft and protease in a salt solution. In some embodiments, the salt solution is a NaCl solution. In some embodiments, the salt solution contains between about 0.15 M NaCl and about 1.5 M NaCl or between about 0.3 M NaCl and about 1.5 M NaCl. In some embodiments, the salt solution contains about 0.3 M NaCl. In some embodiments, step (b) comprises separating the cleaved polypeptide substrate, the uncleaved polypeptide substrate, and the bone graft. In some embodiments, high-performance liquid chromatography is used to separate the cleaved polypeptide substrate, the uncleaved polypeptide substrate, and the bone graft. In some embodiments, size exclusion chromatography is used to separate the cleaved polypeptide substrate, the uncleaved polypeptide substrate, and the bone graft. In some embodiments, another separation method is used to separate the cleaved polypeptide substrate, the uncleaved polypeptide substrate, and/or the bone graft. Other exemplary separation methods include simple centrifugation, ion exchange chromatography, and electrophoresis. In some embodiments, the method for measuring the protease activity associated with a bone graft includes (i) removing at least a portion of the total amount of a protease associated with the bone graft from the bone graft; and (ii) measuring the amount or concentration of one or more proteases removed from the bone graft, thereby determining the amount of protease activity associated with the bone graft. In some embodiments, the polypeptide substrate is PDGF. In some embodiments, the bone graft includes calcium phosphate (such as β-tricalcium phosphate) that has been added to the bone graft. In some embodiments, the bone graft includes one or more other compounds (such as glycerin) that have been added to the bone graft.

In some embodiments of any of the methods, the method for measuring the protease activity associated with a bone graft includes measuring the amount of a polypeptide substrate that is cleaved by a protease activity associated with the bone graft. In some embodiments, measuring the amount of cleaved polypeptide substrate includes (i) incubating the polypeptide substrate with the bone graft, (ii) removing at least a portion of the total amount of cleaved polypeptide substrate from the bone graft, and (iii) measuring the amount of cleaved polypeptide substrate. In some embodiments, the method for measuring the protease activity associated with a bone graft includes (i) incubating PDGF with the bone graft, (ii) removing at least a portion of the total amount of cleaved PDGF from the bone graft, and (iii) measuring the amount of cleaved PDGF. In some embodiments, step (ii) comprises increasing the ionic strength of the solution comprising the bone graft and the polypeptide substrate. In some embodiments, step (ii) comprises incubating the bone graft and the polypeptide substrate in a salt solution. In some embodiments, the salt solution is a NaCl solution. In some embodiments, the salt solution contains between about 0.15 M and about 2.0 M NaCl. In some embodiments, the salt solution contains about 0.6 M NaCl. In some embodiments, step (iii) comprises separating the cleaved polypeptide substrate, the uncleaved polypeptide substrate, and the bone graft. In some embodiments, high-performance liquid chromatography is used to separate the cleaved polypeptide substrate, the uncleaved polypeptide substrate, and the bone graft. In some embodiments, size exclusion chromatography is used to separate the cleaved polypeptide substrate, the uncleaved polypeptide substrate, and the bone graft. In some embodiments, another separation method is used to separate the cleaved polypeptide substrate, the uncleaved polypeptide substrate, and/or the bone graft. Other exemplary separation methods include simple centrifugation, ion exchange chromatography, and electrophoresis. In some embodiments, the polypeptide substrate is PDGF. In some embodiments, the bone graft includes calcium phosphate (such as β-tricalcium phosphate) that has been added to the bone graft. In some embodiments, the bone graft includes one or more other compounds (such as glycerin) that have been added to the bone graft.

In one aspect, the invention features methods for decreasing the protease activity associated with a bone graft (such as a bone allograft). In some embodiments, the method includes removing at least a portion of the total amount of a protease associated with the bone graft from the bone graft. In some embodiments, the method for decreasing the protease activity associated with a bone graft involves removing at least a portion of the total amount of a protease associated with the bone graft from the bone graft by incubating the bone graft in a salt solution containing between about 0.15 M NaCl and about 1.5 M NaCl or between about 0.3 M NaCl and about 1.5 M NaCl. In some embodiments, the method includes measuring the amount of protease that remains associated with the bone graft. In some embodiments, removing the protease comprises increasing the ionic strength of the solution comprising the bone graft and protease. In some embodiments, removing the protease comprises incubating the bone graft and protease in a salt solution. In some embodiments, the salt solution is a NaCl solution. In some embodiments, the salt solution contains between about 0.15 M NaCl and about 1.5 M NaCl or between about 0.3 M NaCl and about 1.5 M NaCl. In some embodiments, the salt solution contains about 0.3 M NaCl. In some embodiments, the bone graft includes calcium phosphate (such as β-tricalcium phosphate) that has been added to the bone graft. In some embodiments, the bone graft includes one or more other compounds (such as glycerin) that have been added to the bone graft. In some embodiments, the method comprises adding a protease inhibitor to the bone graft.

In one aspect, the invention features methods for preparing a bone graft (such as a bone graft or bone allograft for use in the treatment of a bone, periodontium, ligament, cartilage, or tendon condition in an individual). In some embodiments, the method includes the improvement comprising removing at least a portion of the total amount of a protease associated with the a bone graft (such as a bone graft that has undergone one or more treatment steps to make it suitable for use in humans). In some embodiments, removing the protease comprises increasing the ionic strength of a solution comprising the bone graft and the protease. In some embodiments, the method includes the improvement comprising increasing the ionic strength of a solution comprising the bone graft (such as a bone graft that has undergone one or more treatment steps to make it suitable for use in humans) and the protease. In some embodiments, the method includes the improvement comprising washing the bone graft (such as a bone graft that has undergone one or more treatment steps to make it suitable for use in humans) with a salt solution. In some embodiments, the method includes incubating the bone graft and protease in a salt solution. In some embodiments, the salt solution is a NaCl solution, such as between about 0.15 M NaCl and about 1.5 M NaCl or between about 0.3 M NaCl and about 1.5 M NaCl. In some embodiments, the salt solution is about 0.3 M NaCl. In some embodiments, the bone graft includes calcium phosphate (such as β-tricalcium phosphate) that has been added to the bone graft. In some embodiments, the bone graft includes one or more other compounds (such as glycerin) that have been added to the bone graft. In some embodiments, the method comprises adding a protease inhibitor to the bone graft.

In one aspect, the invention provides methods for treating an individual. In some embodiments, the method includes administering a bone graft (such as a bone allograft) and a polypeptide of interest (e.g., PDGF) to an individual. In some embodiments, the bone graft has been selected based on the level of protease activity. In some embodiments, the method involves (a) selecting a bone graft that has an acceptable level of protease activity, and (b) administering the bone graft and a polypeptide of interest to the individual. In some embodiments, the method comprising administering a bone graft and PDGF to an individual, wherein the protease activity of the bone graft is less than about 50 trypsin equivalents. In some embodiments, the method comprising administering a bone graft and PDGF to an individual, wherein the protease activity of the bone graft is between about 50 to about 65 trypsin equivalents (such as about 50 to about 55, about 55 to about 60, or about 60 to about 65 trypsin equivalents). In some embodiments, the method comprising administering a bone graft and PDGF to an individual, wherein the protease activity of the bone graft is about any of 50, 55, 60, or 65 trypsin equivalents. In some embodiments, the method comprises administering a bone graft and PDGF to an individual, wherein the protease activity of the bone graft is less than about 50 trypsin equivalents (such as less than about 45, less that about 40, less than about 35, less that about 30, less than about 25, less than about 20, less than about 15, less than about 10, less than about 5, about 0 trypsin equivalents). In some embodiments, at least a portion of the total amount of a protease associated with the bone graft has been removed from the bone graft prior to administering the bone graft to the individual. In some embodiments, the method includes removing at least a portion of the total amount of a protease associated with the bone graft from the bone graft prior to step (b). In some embodiments, the method comprises adding a protease inhibitor to the bone graft prior to step (b). In some embodiments, the protease activity of two or more bone grafts is measured, and the bone graft with the lowest protease activity is administered to the individual in conjunction with the polypeptide of interest. In some embodiments, the protease activity of the selected bone graft is less than about 50 trypsin equivalents. In some embodiments, the protease activity of the selected bone graft is between about 50 to about 65 trypsin equivalents (such as about 50 to about 55, about 55 to about 60, or about 60 to about 65 trypsin equivalents). In some embodiments, the protease activity of the selected bone graft is about any of 50, 55, 60, or 65 trypsin equivalents. In some embodiments, the protease activity of the selected bone graft is less than about 50 trypsin equivalents (such as less than about 45, less that about 40, less than about 35, less that about 30, less than about 25, less than about 20, less than about 15, less than about 10, less than about 5, about 0 trypsin equivalents).

In some embodiments of any of the methods, selecting the bone graft with an acceptable level of protease activity comprises (i) removing at least a portion of the total amount of a protease associated with the bone graft from the bone graft; and (ii) measuring the amount of a polypeptide substrate that is cleaved by the removed protease, thereby determining the amount of protease activity associated with the bone graft. In some embodiments, step (i) comprises increasing the ionic strength of the solution comprising the bone graft and protease. In some embodiments, step (i) comprises incubating the bone graft and protease in a salt solution. In some embodiments, the salt solution is a NaCl solution. In some embodiments, the salt solution contains between about 0.15 M NaCl and about 1.5 M NaCl or between about 0.3 M NaCl and about 1.5 M NaCl. In some embodiments, the salt solution contains about 0.3 M NaCl. In some embodiments, step (ii) comprises separating the cleaved polypeptide substrate, the uncleaved polypeptide substrate, and the bone graft. In some embodiments, high-performance liquid chromatography is used to separate the cleaved polypeptide substrate, the uncleaved polypeptide substrate, and the bone graft. In some embodiments, size exclusion chromatography is used to separate the cleaved polypeptide substrate, the uncleaved polypeptide substrate, and the bone graft. In some embodiments, another separation method is used to separate the cleaved polypeptide substrate, the uncleaved polypeptide substrate, and/or the bone graft. Other exemplary separation methods include simple centrifugation, ion exchange chromatography, and electrophoresis.

In some embodiments of any of the methods, selecting the bone graft with an acceptable level of protease activity comprises measuring the amount of the polypeptide substrate that is cleaved by a protease activity associated with the bone graft. In some embodiments, measuring the amount of cleaved polypeptide substrate comprises (i) incubating a polypeptide substrate with the bone graft, (ii) removing at least a portion of the total amount of cleaved polypeptide substrate from the bone graft, and (iii) measuring the amount of cleaved polypeptide substrate. In some embodiments, step (ii) comprises increasing the ionic strength of the solution comprising the bone graft and the polypeptide substrate. In some embodiments, step (ii) comprises incubating the bone graft and the polypeptide substrate in a salt solution. In some embodiments, the salt solution is a NaCl solution. In some embodiments, the salt solution contains between about 0.15 M and about 2.0 M NaCl. In some embodiments, the salt solution contains about 0.6 M NaCl. In some embodiments, step (iii) comprises separating the cleaved polypeptide substrate, the uncleaved polypeptide substrate, and the bone graft. In some embodiments, high-performance liquid chromatography is used to separate the cleaved polypeptide substrate, the uncleaved polypeptide substrate, and the bone graft. In some embodiments, size exclusion chromatography is used to separate the cleaved polypeptide substrate, the uncleaved polypeptide substrate, and the bone graft. In some embodiments, another separation method is used to separate the cleaved polypeptide substrate, the uncleaved polypeptide substrate, and/or the bone graft. Other exemplary separation methods include simple centrifugation, ion exchange chromatography, and electrophoresis.

In some embodiments of any of the methods, the polypeptide of interest and the polypeptide substrate are the same. In some embodiments, the polypeptide of interest and the polypeptide substrate are different. In some embodiments, the polypeptide of interest is PDGF. In some embodiments, the bone graft includes calcium phosphate (such as β-tricalcium phosphate) that has been added to the bone graft. In some embodiments, the bone graft includes one or more other compounds (such as glycerin) that have been added to the bone graft.

In some embodiments of any of the methods, the treatment method comprises administering a bone graft and a polypeptide of interest (e.g., PDGF) to an individual. In some embodiments, at least a portion of the total amount of a protease associated with the bone graft has been removed from the bone graft. In some embodiments, the method for treating an individual includes (a) removing at least a portion of the total amount of a protease associated with the bone graft from the bone graft, and (b) administering the bone graft and a polypeptide of interest to the individual. In some embodiments, the method includes administering a bone graft and PDGF to an individual, wherein at least a portion of the total amount of a protease associated with the bone graft has been removed from the bone graft by incubating the bone graft in a salt solution (such as a salt solution that contains between about 0.3 M NaCl and about 1.5 M NaCl). In some embodiments, a protease inhibitor is added to the bone graft.

In some embodiments of any of the methods, the method includes measuring the amount of protease that remains associated with the bone graft. In some embodiments, removing at least a portion of the total amount of a protease associated with the bone graft comprises increasing the ionic strength of the solution comprising the bone graft and protease. In some embodiments, removing at least a portion of the total amount of a protease associated with the bone graft comprises incubating the bone graft and protease in a salt solution. In some embodiments, the salt solution is a NaCl solution. In some embodiments, the salt solution contains between about 0.15 M NaCl and about 1.5 M NaCl or between about 0.3 M NaCl and about 1.5 M NaCl. In some embodiments, the salt solution contains about 0.3 M NaCl. In some embodiments, the polypeptide of interest is PDGF. In some embodiments, the bone graft includes calcium phosphate (such as β-tricalcium phosphate) that has been added to the bone graft. In some embodiments, the bone graft includes one or more other compounds (such as glycerin) that have been added to the bone graft.

In one aspect, the invention features any of the bone grafts (such as bone allografts) described herein for use as a medicament. In some embodiments, the invention features any of the bone grafts described herein in conjunction with any polypeptide of interest for use as a medicament. In some embodiments, the invention features a bone graft described herein in conjunction with any polypeptide of interest for use in a method of treating an individual (such as an individual with a bone, periodontium, ligament, cartilage, or tendon condition). In some embodiments, the invention features the use of any of the bone grafts described herein in conjunction with any polypeptide of interest for the manufacture of a medicament, such as a medicament for treating an individual (such as an individual with a bone, periodontium, ligament, cartilage, or tendon condition).

The invention also features a composition comprising or consisting of bone graft (such as bone allograft) and salt (such as a solution of NaCl). In some embodiments, the composition also comprises a polypeptide of interest (such as PDGF). In some embodiment, the composition includes between about 0.00001 M and about 1.5 M salt, about 0.01 M and about 1.5 M salt, about 0.01 and about 0.15 M salt, about 0.15 M and about 1.5 M salt, or about 0.3 M and about 1.5 M salt. In some embodiment, the composition includes between about 0.00001 M and about 1.5 M NaCl, about 0.01 M and about 1.5 M NaCl, about 0.01 and about 0.15 M NaCl, about 0.15 M and about 1.5 M NaCl, or about 0.3 M and about 1.5 M NaCl.

The invention also features a composition produced by any of the methods described herein, such as a composition comprising bone graft (such as bone allograft) and a salt solution. In some embodiments, the composition also comprises a polypeptide of interest (such as PDGF).

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1 is a summary of an exemplary protocol for studying the binding and release of PDGF from bone graft.

FIG. 2 is a graph showing the PDGF release at 1 hour and 24 hours from bone graft using increasing salt concentrations. The graph shows the amount of PDGF released using PBS solution (far left bar) or increasing concentrations of NaCl solution up to 1.5 M (far right bar) after either 1 hour or 24 hours.

FIG. 3 is a graph showing that PDGF is rapidly eluted from bone graft with 40-60% recovery and that PDGF recovery depends little on mixing time. The mixing times were 10 minutes (left bar), 60 minutes (middle bar), and overnight (right bar) while elution was instant by adding the salt, spinning for 2 minutes at 15,330×g and taking supernatants for analysis. PDGF in a control experiment was taken as 100%. This graph suggests that some PDGF remains associated with the bone graft.

FIG. 4A is a chromatogram showing the use of size exclusion chromatography (SEC) to quantify the amount of PDGF eluted from the human bone graft matrix and to analyze its native size and/or aggregation. SE-HPLC shows the native size of PDGF and its interactions, PDGF aggregation, and other components of the sample. FIG. 4B shows the size exclusion column calibration using protein standards of indicated different molecular sizes.

FIG. 4C is a graph showing the calibration of the SEC column using different concentrations of PDGF.

FIGS. 5A and 5B are chromatograms showing that the SEC profile is temperature and release time dependent. These chromatograms show significant elution of non-specific polypeptides at higher temperatures and longer release times. Elution of PDGF does not change much under the conditions tested.

FIGS. 6A and 6B are chromatograms showing that the SEC profile is sample dependent.

FIG. 7 is a graph showing the protease activity measured using the QuantiCleave™ protease assay (Pierce, Rockford, Ill.). Human bone graft 07-0720-A weighted into 50, 25, and 12.5 mg aliquots in suspension (A), same bone graft incubated with 0.66 M NaCl for 60 minutes at room temperature and then washed three times with 20 mM sodium acetate (AW), same bone graft incubated with 0.66 M NaCl for 60 minutes and then washed with the sodium acetate and protease activity measured in the presence of 5 mM of EDTA (AWE), bone graft supernatant obtained from incubation of the bone graft with 0.66 M NaCl for 60 minutes at room temperature (AWS), the same but assayed in the presence of 5 mM EDTA (AWSE). Data shown are averages of three experiments normalized per mg of dry bone graft.

FIG. 8 is a summary of an exemplary protocol for studying the binding and release of PDGF from bone graft for different lots of bone graft.

FIGS. 9A and 9B are a table and a graph showing that there is no statistically significant age/gender effect of human bone graft on PDGF release.

FIG. 10 is a graph showing the recovery of PDGF from human bone grafts measured by ELISA (left bar) or SEC (right bar) for various bone graft samples. The original amount of PDGF used in the experiment was normalized to 100% compared to the recovered amounts of PDGF.

FIG. 11 is a graph summarizing the PDGF recovery from human bone grafts by ELISA (left bar) or SEC (right bar) for 10 different bone graft samples.

FIG. 12 is a chromatogram showing the use of reversed phase HPLC to quantify the amount of PDGF and to detect changes in its chemical structure due to proteolytic cleavage and/or chemical modification.

FIGS. 13A-13E are chromatograms showing reversed phase HPLC profiles of PDGF released from various bone graft samples (FIGS. 13A-13D) and a PDGF control (FIG. 13E). Triplicate runs are shown. New peaks BC and CD indicate proteolytic cleavage products of PDGF in a case where the bone graft contains proteolytic activity.

FIGS. 14A and 14B is a total ion current (TIC) profile of the PDGF sample and a table showing the identification of PDGF cleavage products by ESI LC/MS.

FIG. 15 is an amino acid sequence of PDGF showing exemplary proteolytic cleavage sites of PDGF isolated from human platelets (Hart et al., Purification of PDGF-AB and PDGF-BB from human platelet extracts and identification of all three PDGF dimers in human platelets Biochemistry, 29:166-172, 1990, which is hereby incorporated by reference in its entirety, particularly with respect to PDGF polypeptides). The same cleavage of PDGF was induced by human bone graft containing a proteolytic activity as shown in FIG. 14.

FIG. 16 is a summary of an exemplary protocol for studying the removal of protease activity from bone graft.

FIGS. 17A-17E are chromatograms showing reversed phase HPLC profiles of PDGF incubated for different times with human bone graft 07-0720-A extract (supernatant).

FIGS. 18A and 18B are graphs showing the time dependence of PDGF proteolytic cleavage.

FIGS. 19A-19C are chromatograms showing reversed phase HPLC profiles of PDGF incubated for different times with solid human bone graft 07-0720-A after 0.3 M salt elution.

FIG. 20 is a graph showing the cumulative release of PDGF from DMFDBA following 5 minute washes with sterile water (left bar), sterile saline (center bar), or sterile elution buffer (right bar).

FIGS. 21A-21D show molecular function comparison of the proteins/peptides contained in various allograft lots.

FIG. 22 shows a comparison of the top proteins comprising 5% or more of any one allograft in various allograft lots.

### DETAILED DESCRIPTION OF THE INVENTION

The present invention is based in part on the surprising discovery that bone grafts (such as human bone allografts) can have residual protease activity even after they have been treated to reduce the amount of endogenous polypeptides to minimize adverse reactions when transplanted into humans. In particular, it was discovered that the amount of residual protease activity associated with human bone grafts is variable. This residual protease activity is undesirable for bone grafts that are administered in conjunction with a polypeptide of interest (such as PDGF) to an individual because the protease activity can cleave the polypeptide of interest (either before or after the polypeptide of interest is administered to the individual). The cleavage of the polypeptide of interest produces variability in the structure of the polypeptide of interest because a mixture of full length and cleaved polypeptide is produced. In addition, the percentage of cleaved polypeptide may increase over time. In contrast, a more uniform composition of the polypeptide of interest is desirable to minimize or prevent changes in biological activity or stability that may occur due to changes in the structure of the polypeptide of interest. In particular, it is desirable to minimize changes to the structure (e.g., its primary, secondary, or tertiary structure) and/or function of a polypeptide of interest due to interaction with a bone graft matrix for therapeutic methods involving the administration of the polypeptide of interest and a bone graft to an individual (such as a human with a bone, periodontium, ligament, cartilage, or tendon condition).

The invention also provides methods of measuring the protease activity associated with a bone graft. This measurement enables one to determine whether a particular bone graft should be administered to an individual (such as a human with a bone, periodontium, ligament, cartilage, or tendon condition) in combination with a polypeptide of interest. Additionally, the invention features methods of reducing the level of protease activity associated with a bone graft. These methods allow bone grafts with an acceptable level of protease activity (or no protease activity) to be generated for therapeutic applications.

In the following sections, exemplary bone grafts, polypeptides of interest, and polypeptide substrates are first described. Then, exemplary methods for characterizing bone grafts and/or selecting bone grafts with desirable properties are explained. Next, methods for decreasing the level of protease activity associated with a bone graft are described. Exemplary treatment methods and kits are then disclosed.

### Exemplary Bone Grafts

Exemplary bone grafts include bone allografts, isografts, autografts, and xenografts. Bone allografts include bone or bone cells from a donor that can be transplanted into a genetically non-identical member of the same species. Transplanted bone or bone cells from a genetically identical donor, i.e., an identical twin, is termed an isograft. When a cell or tissue is transplanted from one site to another in the same individual, it is termed an autograft. In contrast, a transplant from another species is called a xenograft. In some cases, bone from a human donor is transplanted into another human. Exemplary human bone allografts are pieces of bone skeleton isolated post mortem from human donors. The bone graft may be mineralized or partially or completely demineralized using standard methods (˜4% residual is usually the most demineralization used). In particular embodiments, the bone graft is non-demineralized. In particular embodiments, the bone grafts is deorganified using standard methods. In particular embodiments, the bone grafts is non-demineralized and deorganified. In some embodiments, the bone graft contains a combination of (i) mineralized bone and (ii) partially or completely demineralized bone. If desired, the bone graft may be partially or completely deproteinized using standard methods, such as a deproteinized bovine or human bone block. Exemplary bone grafts include a demineralized freeze-dried bone graft (DFDBA), a freeze-dried bone graft (FDBA), a fresh frozen bone allograft, a particulate demineralized bone matrix (DBM), or a bone block (see, for example, U.S. Ser. No. 60/890,763, filed Feb. 20, 2007; U.S. Pub. No. 2007/0207185, filed Feb. 9, 2007; U.S. Pub. No. 2007/0129807; filed Nov. 17, 2006, which are each hereby incorporated by reference in their entireties, particularly with respect to bone grafts). In some embodiments, the bone graft is an autologous cortical, cancellous, or cortico-cancellous bone block. In some embodiments, the bone graft is a deorganified xenogeneic material, e.g., BioOss (Geistlich Biomaterials, Inc.). In some embodiments, the bone graft is a commercially prepared bone graft for use in humans. In some embodiments, the bone graft has been treated so that it is suitable for use in humans. Exemplary treatment steps to make a bone graft suitable for use in humans include, but are not limited to, bioburden control, bioburden assessment, minimized contamination, rigorous cleaning, disinfection and rinsing, milling, freeze drying, aliquotting, packaging, terminal sterilization, mineralization, demineralization, freeze drying, aseptic preparation, bone block or granulate formation, or any combination of two or more of the foregoing. In some embodiments, the bone graft undergoes Allowash XG™ (bioburden control, bioburden assessment, minimized contamination, rigorous cleaning, disinfection, and rinsing). In various embodiments, the bone allograft is milled or not milled, freeze-dried or not freeze-dried, sterilized or just aseptically prepared. In some embodiments, the bone graft is treated with one or more chemicals (such as hydrogen peroxide, detergent surfactants such as nonoxynyl-9, or isopropyl alcolol) or antibiotics (such as polymyxin or bacitracin). In some embodiments, the bone graft is exposed either to gamma radiation or to ethylene oxide for sterilization. Not all bone grafts are terminally sterilized. In some embodiments, the bone graft is treated to remove viruses and/or bacteria.

In some embodiments, the bone graft has been washed (such as washed in water, saline, or elution buffer) prior to the addition of a polypeptide of interest. Exemplary bone grafts are derived from one or more of the following types of bone: humerus, ulna, radius, femur, tibia, fibula, patella, ankle bones, wrist bones, carpals, metacarpals, phalanges, tarsals, metatarsals, ribs, sternum, vertebrae, scapula, clavicle, pelvis, sacrum, and craniofacial bones. Exemplary donors for bone grafts include a primate (e.g., a human, monkey, gorilla, ape, lemur, etc.), a bovine, an equine, a porcine, an ovine, a canine, and a feline.

In some embodiments, the bone graft includes particles, blends, meshes, or blocks. Any appropriate overall size of bone graft can be used, such as an overall size useful to treat a bone defect or injury of a particular size. In some embodiments, the bone graft consists of particulates of any appropriate size, such as between about 50 and about 750 um, about 50 and about 500 um, about 125 and about 500 um, about 250 and about 710 um, or about 125 and about 1000 um. In some embodiments, the bone graft consists of a bone block with an average diameter between about 50 um and about 100 mm or about 0.1 mm and about 100 mm. In some embodiments, the particulates are less than about 100 um (such as between about 50 and about 90 um) or greater than 355 um (such as between about 360 and about 1000 um) since bone graft with a particle size between about 100 and about 355 um may be less flowable than desired for some applications. Flowability refers to the ability to pass the material through a cannula or small gauge tube as a homogeneous mixture, that is, without the separation of the liquid from the particulate. In some embodiments, a broad size range (such as between about 250 and about 710 um) is used to maximize the yield from the bone graft. For example, ground cortical bone is processed on standard grinding equipment that produces particulate in a range of sizes. The broader the size range that is allowable, the greater the yield.

In particular embodiments, the bone graft comprises or consists of a ratio of about 1:1 of freeze dried ground cortical bone to demineralized freeze dried ground cortical bone. In some such embodiments, no exogenous calcium phosphate is added.

Porous bone grafts, according to some embodiments, can comprise pores having diameters ranging from about 1 um to about 1 mm. In one embodiment, a bone graft comprises macropores having diameters ranging from about 100 um to about 1 mm. In another embodiment, a bone graft comprises mesopores having diameters ranging from about 10 um to about 100 um. In a further embodiment, a bone graft comprises micropores having diameters less than about 10 um. Embodiments of the present invention contemplate bone grafts comprising macropores, mesopores, micropores, or any combination thereof. A porous bone graft, in one embodiment, includes a bone graft with a porosity of about or greater than any of 25, 30, 40, 50, 60, 70, 75, 80, 85, 90, or 95%. In some embodiments, the porous structure of the bone graft allows for infiltration of cells (such as osteoblasts) into pores of the matrix. In some embodiments, a bone graft comprises a porous structure having multidirectional and/or interconnected pores. In other embodiments, a bone graft comprises a porous structure having pores that are not interconnected. In some embodiments, a bone graft comprises a porous structure having a mixture of interconnected pores and pores that are not interconnected. In some embodiments, a bone graft is porous and able to absorb water in an amount ranging from about 1 to about 15 times the mass of the bone graft.

In some embodiments, the bone graft includes calcium phosphate that has been added to the bone graft (such as exogenous calcium phosphate). For example, any of the calcium phosphates disclosed in U.S. Pub. No. 2007/0207185, filed Feb. 9, 2007, can be used (which is hereby incorporated by reference in its entirety, particularly with respect to calcium phosphates). Calcium phosphates suitable for use in conjunction with a bone graft, in some embodiments of the present invention, have a calcium to phosphorus atomic ratio ranging from about 0.5 to about 2.0. Non-limiting examples of calcium phosphates suitable for use in conjunction with a bone graft comprise amorphous calcium phosphate, monocalcium phosphate monohydrate (MCPM), monocalcium phosphate anhydrous (MCPA), dicalcium phosphate dihydrate (DCPD), dicalcium phosphate anhydrous (DCPA), octacalcium phosphate (OCP), α-tricalcium phosphate, β-tricalcium phosphate (β-TCP), hydroxyapatite (OHAp), poorly crystalline hydroxyapatite, tetracalcium phosphate (TTCP), heptacalcium decaphosphate, calcium metaphosphate, calcium pyrophosphate dihydrate, carbonated calcium phosphate, and calcium pyrophosphate. In some embodiments, the matrix includes about any of 1, 2, 3, 4, 5, or 6 times more bone graft by weight than the weight of added calcium phosphate, such as β-TCP. In some embodiments, the matrix includes about 80% by weight bone graft (such as bone allograft) and about 20% by weight of another calcium phosphate, such as β-TCP. In some embodiments, the calcium phosphate (such as β-TCP) has a porosity of about or greater than any of 40, 50, 60, 70, 75, 80, 85, 90, or 95%.

In some embodiments, a biocompatible binder is added to the bone graft (such as bone graft alone or a mixture of a bone graft and an exogenous calcium phosphate). For example, any of the biocompatible binders disclosed in U.S. Pub. No. 2007/0207185, filed Feb. 9, 2007, can be used (which is hereby incorporated by reference in its entirety, particularly with respect to biocompatible binders). Biocompatible binders, in some embodiments, can comprise collagen, elastin, polysaccharides, nucleic acids, carbohydrates, proteins, polypeptides, poly(α-hydroxy acids), poly(lactones), poly(amino acids), poly(anhydrides), polyurethanes, poly(orthoesters), poly(anhydride-co-imides), poly(orthocarbonates), poly(α-hydroxy alkanoates), poly(dioxanones), poly(phosphoesters), polylacetic acid, poly(L-lactide) (PLLA), poly(D,L-lactide) (PDLLA), polyglycolide (PGA), poly(lactide-co-glycolide (PLGA), poly(L-lactide-co-D,L-lactide), poly(D,L-lactide-co-trimethylene carbonate), polyglycolic acid, polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate)polyamide, and copolymers and mixtures thereof. Biocompatible binders, in other embodiments, can comprise alginic acid, arabic gum, guar gum, xantham gum, gelatin, chitin, chitosan, chitosan acetate, chitosan lactate, chondroitin sulfate, N,O-carboxymethyl chitosan, a dextran (e.g., α-cyclodextrin, β-cyclodextrin, γ-cyclodextrin, or sodium dextran sulfate), fibrin glue, lecithin, phosphatidylcholine derivatives, glycerol, hyaluronic acid, sodium hyaluronate, a cellulose (e.g., methylcellulose, carboxymethylcellulose, hydroxypropyl methylcellulose, or hydroxyethyl cellulose), a glucosamine, a proteoglycan, a starch (e.g., hydroxyethyl starch or starch soluble), lacetic acid, pluronic acids, sodium glycerophosphate, glycogen, a keratin, silk, and derivatives and mixtures thereof.

In some embodiments, the porous structure of the bone graft allows for release of greater than or about any of 20, 30, 40, 50, 60, or 70% of a polypeptide of interest (such as PDGF) after about 1 hour (based on the amount of the polypeptide of interest measured using an appropriate assay such as an ELISA or size exclusion chromatography assay described herein). In other embodiments, the porous structure of the bone graft allows for release of greater than or about any of 20, 30, 40, 50, 60, or 70% of the polypeptide of interest (such as PDGF) after about 8 hours. In other embodiments, the porous structure of the bone graft allows for release of greater than or about any of 20, 30, 40, 50, 60, or 70% of the polypeptide of interest (such as PDGF) after about 24 hours.

In some embodiments, the bone graft is bioresorbable. “Bioresorbable” refers to the ability of a bone graft to be resorbed or remodeled in vivo. The resorption process involves degradation and elimination of the original material through the action of body fluids, enzymes, or cells. The resorbed material may be used by the treated individual in the formation of new tissue, or it may be otherwise re-utilized by the treated individual, or it may be excreted. A bone graft, in some embodiments, can be resorbed within one year of in vivo administration. In other embodiments, a bone graft can be resorbed within 1, 3, 6, or 9 months of in vivo administration. Bioresorbability is dependent on: (1) the nature of the matrix material (i.e., its chemical make up, physical structure, and size); (2) the location within the body in which the matrix is placed; (3) the amount of matrix material that is used; (4) the metabolic state of the individual being treated (diabetic/non-diabetic, osteoporotic, smoker, age, steroid use, etc.); (5) the extent and/or type of injury or condition treated; and (6) the use of other materials in addition to the matrix such as other bone anabolic, catabolic, and anti-catabolic factors.

### Exemplary Polypeptides of Interest

Any polypeptide of interest can be used with the bone grafts described herein. The terms “polypeptide” and “protein” are used interchangeably to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In some embodiments, the polypeptide of interest is a polypeptide capable of being cleaved by one or more proteases associated with a bone graft. If desired, the ability of a polypeptide of interest to be cleaved by one or more proteases associated with a bone graft can be measured using any of the methods described herein (such as by incubating the polypeptide of interest with a bone graft, separating the polypeptide of interest from the bone graft, and measuring the amount of the polypeptide of interest that was cleaved). Desirably, the methods described herein reduce the amount of a protease associated with a bone graft that can cleave the polypeptide of interest. In some embodiments, more than one polypeptide of interest (such as 2, 3, 4, 5, or more different polypeptides) are used in the methods, compositions, or kits described herein. In some embodiments, the polypeptide of interest promotes bone repair, healing, or growth.

In some embodiments, the polypeptide of interest is PDGF, which is a growth factor naturally released from platelets at sites of injury. PDGF synergizes with VEGF to promote neovascularization, and stimulates chemotaxis and proliferation of mesenchymally-derived cells including tenocytes, osteoblasts, chondrocytes, and vascular smooth muscle cells. In some embodiments, PDGF comprises PDGF homodimers and heterodimers, including PDGF-AA, PDGF-BB, PDGF-AB, PDGF-CC, PDGF-DD, and mixtures and derivatives thereof. In some embodiments, PDGF comprises PDGF-BB. In other embodiments, PDGF comprises a recombinant human PDGF, such as rhPDGF-BB. In some embodiments, PDGF comprises PDGF fragments. In one embodiment, rhPDGF-B comprises the following fragments: amino acid sequences 1-31, 1-32, 33-108, 33-109, and/or 1-108 of the entire B chain. The complete amino acid sequence (amino acids 1-109) of the B chain of PDGF is provided in FIG. 15 of U.S. Pat. No. 5,516,896 (which is hereby incorporated by reference in its entirety, particularly with respect to PDGF polypeptides). It is to be understood that the rhPDGF compositions of the present invention may comprise a combination of intact rhPDGF-B (amino acids 1-109) and fragments thereof. Other fragments of PDGF may be employed such as those disclosed in U.S. Pat. No. 5,516,896. In accordance with some embodiments, the rhPDGF-BB comprises greater than or about any of 65%, 75%, 80%, 85%, 90%, 95%, or 99% of intact rhPDGF-B (amino acids 1-109).

In some embodiments, the polypeptide of interest is a polypeptide that is cleaved by one or more proteases (such as aminopeptidases, carboxypeptidases, and metalloproteases) associated with a bone graft that also cleaves PDGF. For example, the polypeptide of interest can have one or more of the cleavage sites shown in FIG. 15 for PDGF: cleavage of the peptide bond after Ser1, Leu5, or Arg32. In some embodiments, the polypeptide of interest contains at least about any of 2, 3, 4, 5, 6, 7, or more contiguous amino acids that are identical to 2, 3, 4, 5, 6, 7, or more contiguous amino acids of a PDGF polypeptide that include Ser1, Leu5, or Arg32. In some embodiments, the polypeptide of interest contains at least about any of 4, 5, 6, 7, or more contiguous amino acids that are greater than or about 80, 85, 95, 99, or 100% identical to contiguous amino acids of a PDGF polypeptide that include Ser1, Leu5, or Arg32. Sequence identity can be measured, for example, using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various amino acids replacements, deletions, and other modifications.

In some embodiments, the polypeptide of interest is obtained from natural sources. In other embodiments, the polypeptide of interest is produced by recombinant DNA techniques. In some embodiments, the polypeptide of interest or fragments thereof may be produced using peptide synthesis techniques known to one of skill in the art, such as solid phase peptide synthesis.

When obtained from natural sources, the polypeptide of interest can be, for example, derived from biological fluids. Biological fluids, according to some embodiments, can comprise any treated or untreated fluid associated with living organisms, including blood. Biological fluids can also comprise blood components including platelet concentrate, apheresed platelets, platelet-rich plasma, plasma, serum, fresh frozen plasma, and buffy coat. Biological fluids can comprise platelets separated from plasma and resuspended in a physiological fluid.

When produced by recombinant DNA techniques, a DNA sequence encoding a single monomer (e.g., PDGF B-chain or A-chain) can be inserted into cultured prokaryotic or eukaryotic cells for expression to subsequently produce the homodimer (e.g., PDGF-BB or PDGF-AA). The homodimer PDGF produced by recombinant techniques may be used in some embodiments. In some embodiments, a homodimer of PDGF is produced in engineered yeast cells such as Saccharomyces cerevisiae. In other embodiments, a PDGF heterodimer can be generated by inserting DNA sequences encoding for both monomeric units of the heterodimer into cultured prokaryotic or eukaryotic cells and allowing the translated monomeric units to be processed by the cells to produce the heterodimer (e.g., PDGF-AB). Commercially available recombinant polypeptides of interest (such as human PDGF-BB) may be obtained from a variety of sources.

In some embodiments, the polypeptide of interest is in a highly purified form. “Purified polypeptide,” as used herein, comprises compositions having greater than or about 95% by weight of the polypeptide of interest prior to incorporation into solutions of the present invention. The solution may be prepared using any pharmaceutically acceptable buffer or diluent. In other embodiments, the polypeptide of interest can be substantially purified. “Substantially purified polypeptide,” as used herein, comprises compositions having about 5% to about 95% by weight of the polypeptide of interest prior to incorporation into solutions of the present invention. In one embodiment, substantially purified polypeptide comprises compositions having about 65% to about 95% by weight of the polypeptide of interest prior to incorporation into solutions of the present invention. In other embodiments, substantially purified polypeptide of interest comprises compositions having about 70% to about 95%, about 75% to about 95%, about 80% to about 95%, about 85% to about 95%, or about 90% to about 95%, by weight of the polypeptide of interest, prior to incorporation into solutions of the present invention. Purified polypeptide of interest and substantially purified polypeptide of interest may be incorporated into the bone graft.

In a further embodiment, the polypeptide of interest can be partially purified. Exemplary partially purified polypeptides (such as PDGF) comprise compositions having the polypeptide of interest in the context of platelet-rich plasma, fresh frozen plasma, or any other blood product that requires collection and separation to produce the polypeptide of interest. Embodiments of the present invention contemplate that any of the polypeptide isoforms provided herein, including homodimers and heterodimers, can be purified or partially purified. Compositions of the present invention comprising polypeptide mixtures may comprise isoforms, variants, or fragments of the polypeptide of interest in partially purified proportions. Partially purified and purified PDGF, in some embodiments, can be prepared as described in U.S. Pub. No. 2006/0084602, filed Jun. 23, 2005 (which is hereby incorporated by reference in its entirety, particularly with respect to PDGF polypeptides).

### Exemplary Polypeptide Substrates

Any polypeptide capable of being cleaved by a protease can be used as a polypeptide substrate in any of the methods described herein for measuring the protease activity associated with a bone graft or selecting a bone graft with an acceptable level of protease activity. Exemplary polypeptide substrates include any of the polypeptides of interest described herein. In some embodiments, the polypeptide substrate is a polypeptide known to be cleaved by one or more proteases (such as aminopeptidases, carboxypeptidases, and/or metalloproteases). In some embodiments, the polypeptide substrate is a commercially available polypeptide (e.g., succinylated casein in the QuantiCleave™ protease assay kit (Pierce, Eockfrd, Ill.), bovine hemoglobin (cat. # H2625, Sigma-Aldrich, St. Louis, Mo.), gelatin, (Cat. # G7765, Sigma, St Louis, Mo.), or a casein fluorescein isotiocyanate from bovine milk Type I (Cat. # C0403, Sigma-Aldrich, St. Louis, Mo.)).

Exemplary Methods for Characterizing and/or Selecting Bone Grafts

If desired, any of the bone grafts described herein can be analyzed to determine the amount of protease activity (such as the activity of one or more aminopeptidases, carboxypeptidases, and/or metalloproteases) associated with the bone graft (for example, to predict how much of a polypeptide of interest will be cleaved by the protease activity associated with the bone graft when the polypeptide of interest is administered in conjunction with the bone graft). In one such aspect, the invention features methods for measuring the protease activity associated with a bone graft. In some embodiments, the method includes measuring the amount of a polypeptide substrate (such as PDGF) that is cleaved by a protease activity associated with the bone graft. In some embodiments, measuring the amount of cleaved polypeptide substrate includes (i) incubating the polypeptide substrate with the bone graft, (ii) removing at least a portion of the total amount of cleaved polypeptide substrate from the bone graft, and (iii) measuring the amount of cleaved polypeptide substrate. In some embodiments, step (ii) comprises increasing the ionic strength of the solution comprising the bone graft and the polypeptide substrate. In some embodiments, step (ii) comprises incubating the bone graft and the polypeptide substrate in a salt solution. In some embodiments, the salt solution is a NaCl solution, such as between about 0.15 M and about 2.0 M NaCl or about 0.6 M NaCl. In some embodiments, step (iii) comprises separating the cleaved polypeptide substrate, the uncleaved polypeptide substrate, and the bone graft. In some embodiments, high-performance liquid chromatography (HPLC) is used to separate the cleaved polypeptide substrate, the uncleaved polypeptide substrate, and the bone graft. In some embodiments, size exclusion chromatography (SEC) is used to separate the cleaved polypeptide substrate, the uncleaved polypeptide substrate, and the bone graft. In some embodiments, the polypeptide substrate is PDGF.

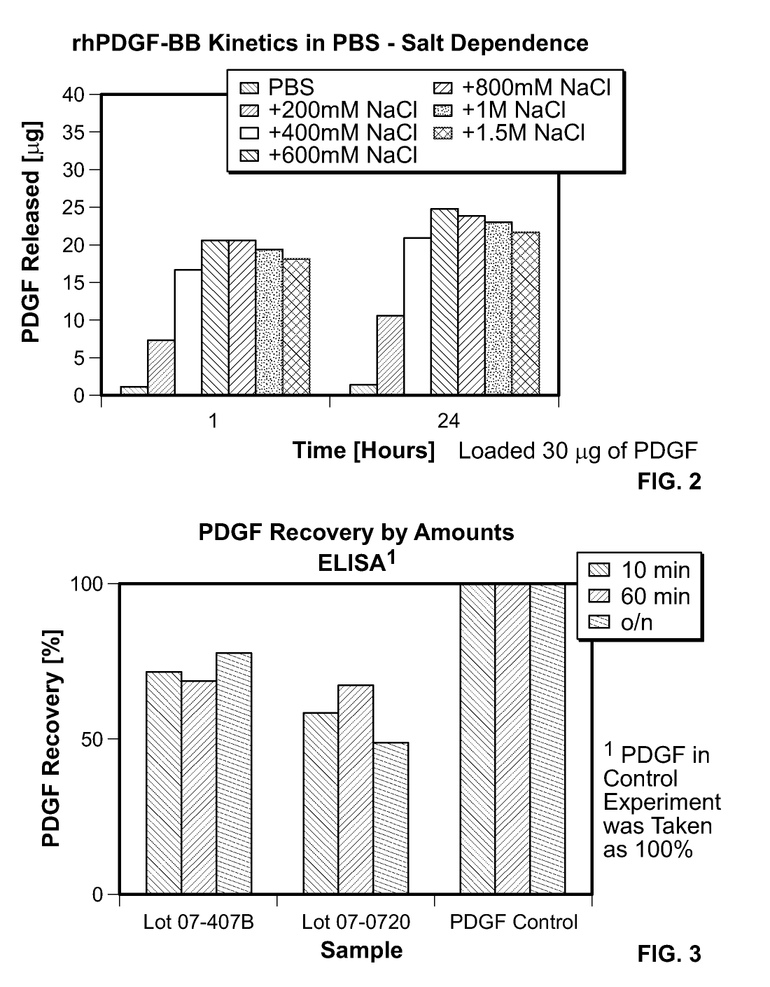

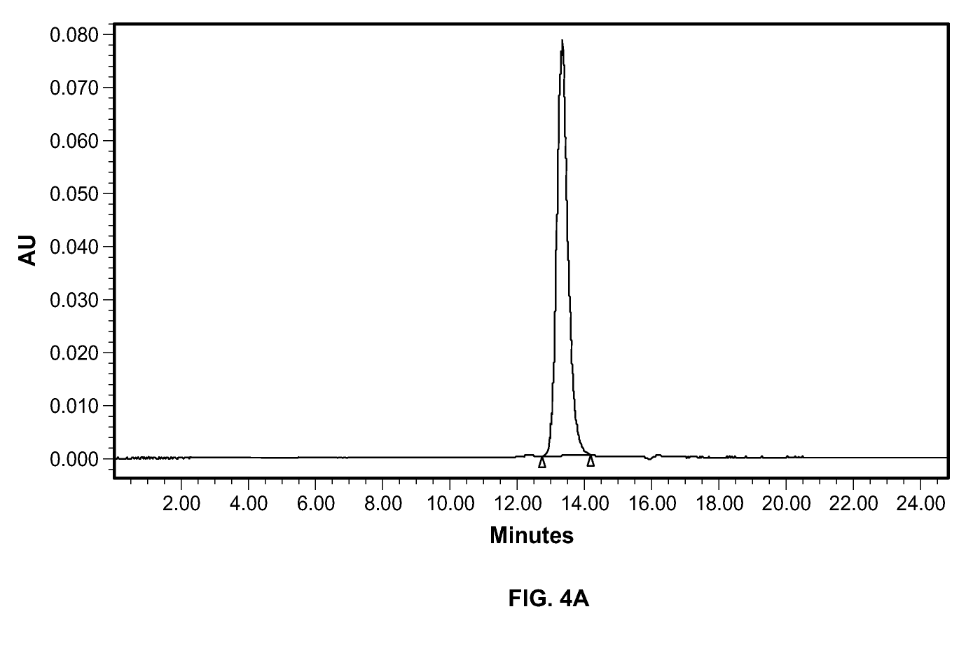

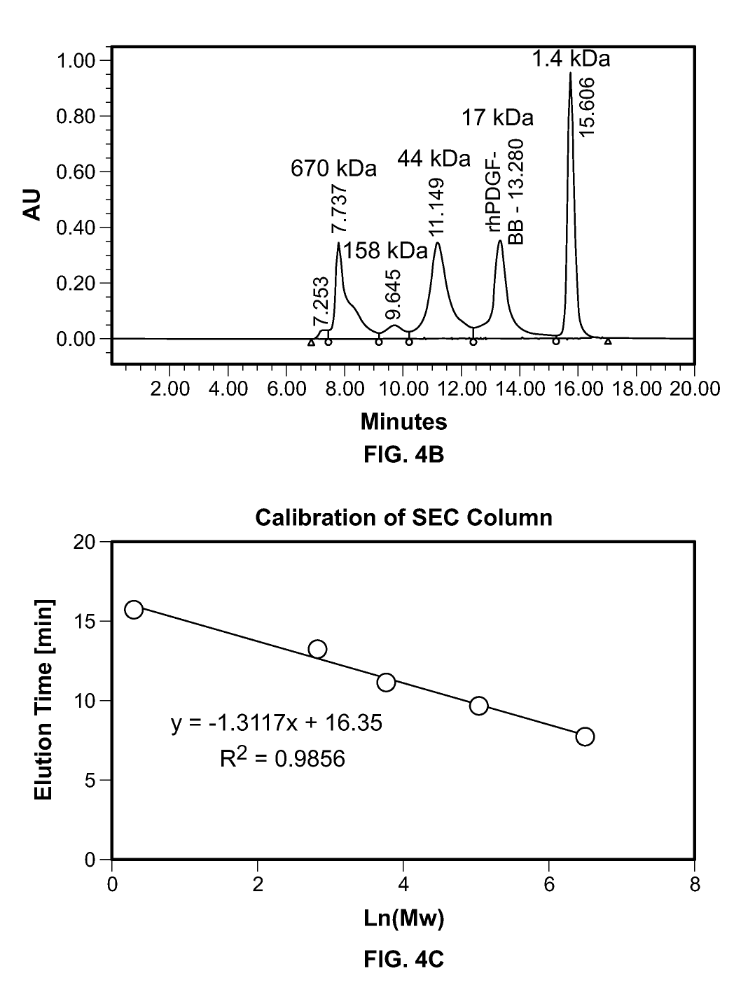

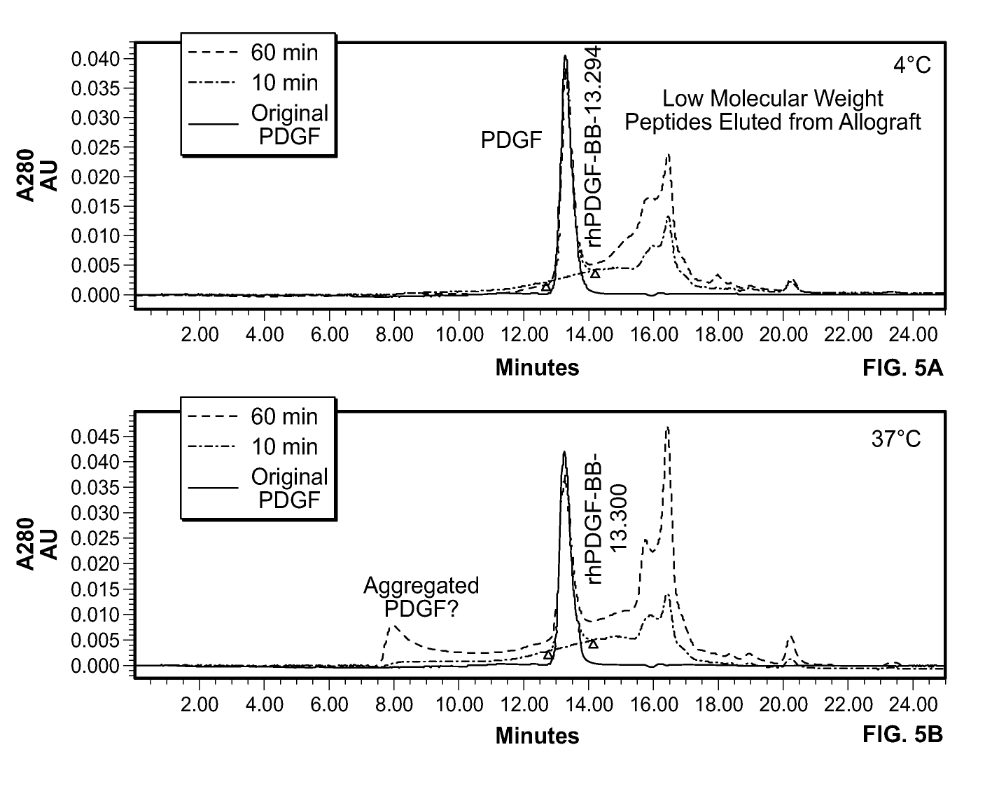

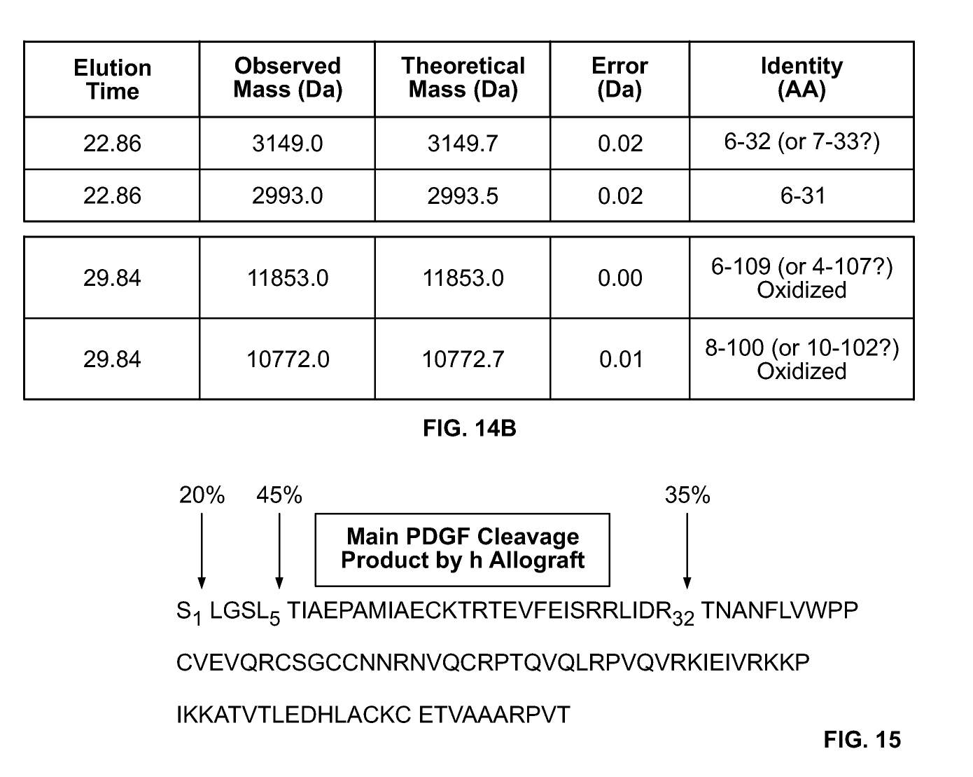

As described further in the Examples, methods were developed to separate PDGF or another polypeptide of interest after it binds a bone graft. For example, when a mixture of rhPDGF-BB and bone graft was washed with a low ionic strength buffer, less than 10% of the rhPDGF-BB that had bound to the bone graft matrix was recovered from the bone graft (by release of the rhPDGF-BB from the bone graft into the solution). Increasing the ionic strength of the buffer increased the amount of rhPDGF-BB that was released from the bone graft (FIG. 2). The optimal concentration of salt (such as NaCl) was 0.6 M, although other concentrations such as between about 0.15 M and about 2.0 M can be used. The release of the rhPDGF-BB from the matrix was almost instantaneous under these conditions (FIG. 3). Other monovalent or bivalent salts can be used to achieve release of rhPDGF or another polypeptide of interest from bone graft such as KCl, LiCl, (NH₄)₂SO₄, NaHPO₄, etc. If desired, an ELISA assay can be used to measure the amount of PDGF or other polypeptide of interest (such as the amount of soluble polypeptide) that is released from the bone graft. The ELISA assay described in the Examples measures the binding of PDGF to its receptor, allowing the amount of PDGF that is still able to bind its receptor to be measured. The Examples describe reverse phase HPLC (RPHPLC) and SEC methods, e.g., high performance size exclusion chromatography (HPSEC) that were used to separate cleaved PDGF and uncleaved PDGF from the bone graft, allowing the percentage of PDGF that was cleaved by a protease associated with the bone graft to be determined. For example, these methods allowed changes in the structure (such as cleavage or chemical modification) and the extent of polypeptide aggregation to be determined. For example, if PDGF aggregates, its size increases and it elutes at earlier elution times as shown in FIG. 5B at 37° C. Any changes in the RPHPLC profile (such as the appearance of new peaks) indicate a possible change in the structure of rhPDGF-BB, most likely due to a proteolytic cleavage or chemical modification of some amino acid residues or both. These methods also allowed separation of rhPDGF-BB from endogenous bone graft polypeptides that may otherwise hinder the analysis of rhPDGF-BB. To determine the amount of rhPDGF-BB using the SEC method, the peaks corresponding to rhPDGF-BB were integrated, and the rhPDGF-BB concentration was determined by using a calibration curve calculated from rhPDGF-BB standards of known concentrations as shown in FIGS. 4A-4C. To determine the amount of rhPDGF-BB using the RPHPLC method, the sum of all peak areas belonging to the components of rhPDGF-BB is used. Indeed, some bone grafts induced proteolytic cleavage of rhPDGF-BB, as demonstrated by the appearance of new peaks in the RPHPLC profile (FIG. 14A). Because the new peaks can be separated in the RPHPLC profile, the RPHPLC method can be used for identification of the new polypeptide peaks by mass spectrometry (FIG. 14B) or any other standard method (e.g., Edman N-terminal sequencing). Thus, cleavage sites in rhPDGF-BB (FIG. 15) or another polypeptide of interest and/or the protease(s) causing the cleavage can be identified. If desired, functional properties of the polypeptide of interest can be measured after it is released from the bone graft using standard cell-based assays, such as assays that measure cell proliferation in response to incubation with a growth factor of interest (e.g., cell-based alkaline phosphatase bioassays). For example, a bioassay measuring the stimulatory effect of rhPDGF-BB on the growth of MG-63 cells can be used.

In some embodiments, the method for measuring the protease activity associated with a bone graft includes (a) removing at least a portion of the total amount of a protease associated with the bone graft from the bone graft; and (b) measuring the amount of a polypeptide substrate that is cleaved by the removed protease, thereby determining the amount of protease activity associated with the bone graft. In some embodiments, step (a) comprises increasing the ionic strength of the solution comprising the bone graft and protease. In some embodiments, step (a) comprises incubating the bone graft and protease in a salt solution. In some embodiments, the salt solution is a NaCl solution, such as between about 0.15 M NaCl and about 1.5 M NaCl or about 0.3 M NaCl and about 1.5 M NaCl. In some embodiments, step (b) comprises separating the cleaved polypeptide substrate, the uncleaved polypeptide substrate, and the bone graft. In some embodiments, HPLC or SEC is used to separate the cleaved polypeptide substrate, the uncleaved polypeptide substrate, and the bone graft. In some embodiments, the method for measuring the protease activity associated with a bone graft includes (i) removing at least a portion of the total amount of a protease associated with the bone graft from the bone graft; and (ii) measuring the amount or concentration of one or more proteases removed from the bone graft, thereby determining the amount of protease activity associated with the bone graft. In some embodiments, the polypeptide substrate is PDGF.

As described further in the Examples, methods were developed to remove at least a portion of the protease activity from a bone graft. The protease activity was almost completely eluted from human bone graft using 0.3 M NaCl, although other concentrations of salt can be used, such as between about 0.15 M and about 1.5 M. Eighty minutes was the optimal time for assaying protease activity at 37° C. using PDGF as the substrate, although other incubation times and temperatures can be used. If desired, standard Edman N-terminal sequencing can be used to identify the protease after it is removed from the bone graft. Alternatively or additionally, mass spectrometry can be used to determine the size and identity of the protease after it is removed from the bone graft using standard methods such as those described herein.

In one aspect, the invention features methods of selecting a bone graft for administration to an individual in conjunction with a polypeptide of interest (e.g., PDGF). In some embodiments, the method involves measuring the protease activity associated with a bone graft, whereby the amount of protease activity associated with the bone graft determines whether the bone graft is selected for administration to the individual in conjunction with the polypeptide of interest. In some embodiments, the method involves selecting a bone graft with an acceptable level of protease activity for administration to the individual in conjunction with the polypeptide of interest. In some embodiments, the protease activity of two or more bone grafts is measured, and the bone graft with the lowest protease activity is administered to the individual in conjunction with the polypeptide of interest. In some embodiments, the protease activity of the selected bone graft is less than about 50 trypsin equivalents. In some embodiments, the protease activity of the selected bone graft is between about 50 to about 65 trypsin equivalents (such as about 50 to about 55, about 55 to about 60, or about 60 to about 65 trypsin equivalents). In some embodiments, the protease activity of the selected bone graft is about any of 50, 55, 60, or 65 trypsin equivalents. In some embodiments, the protease activity of the selected bone graft is less than about 50 trypsin equivalents (such as less than about 45, less that about 40, less than about 35, less that about 30, less than about 25, less than about 20, less than about 15, less than about 10, less than about 5, about 0 trypsin equivalents). In some embodiments, the method also involves selecting a bone graft that binds an acceptable amount of the initial polypeptide of interest and/or that releases an acceptable percentage of the polypeptide of interest that bound to the bone graft.

Exemplary Methods for Decreasing the Protease Activity Associated with a Bone Graft

In one aspect, the invention features methods for decreasing the protease activity associated with a bone graft. In some embodiments, the method includes removing at least a portion of the total amount of a protease associated with the bone graft from the bone graft. In some embodiments, removing the protease comprises increasing the ionic strength of the solution comprising the bone graft and protease. In some embodiments, removing the protease comprises incubating the bone graft and protease in a salt solution (such as a NaCl solution). In some embodiments, the salt solution contains between about 0.15 M NaCl and about 1.5 M NaCl or between about 0.3 M NaCl and about 1.5 M NaCl. In some embodiments, the salt solution contains about 0.3 M NaCl. In some embodiments, the method includes measuring the amount of protease that remains associated with the bone graft. In some embodiments, the bone graft retains at least a portion of its osteoinductive activity after the protease is removed. In some embodiments, the bone graft retains at least a portion of the endogenous polypeptides associated with it (such as BMPs) after the protease is removed. In some embodiments, the bone graft is then administered to an individual as described below.

In some embodiments, a protease inhibitor may be added to the bone graft. For example, a protease inhibitor(s) specific to one or more proteases in the bone graft may be added, e.g. a protease inhibitor for cathepsin G, matrix metalloprotease-9, and/or chymase.

### Exemplary Methods for Washing Bone Graft

In some embodiments, the bone graft (such as human bone allograft) is washed (such as washed in water, saline, or elution buffer) prior to the addition of a polypeptide of interest. This washing step may be in addition to or instead of the removal of a portion of the protease activity from the bone graft. In some embodiments, this washing step removes an acidic residue from the bone graft. In some embodiments, this washing step improves the ability of the bone graft to retain a polypeptide of interest.

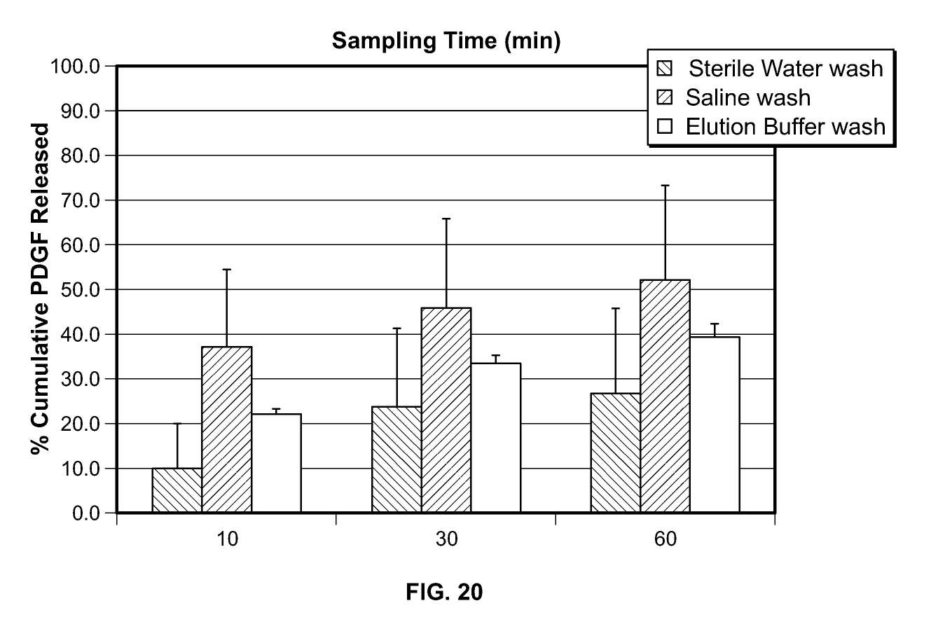

For example, FIG. 20 summarizes how washing demineralized human bone graft in either water, saline, or an elution buffer affects the binding of PDGF to the bone graft. The trends indicated that washing the bone graft for 5 minutes in sterile water led to a decrease in the amount of PDGF released from DM bone graft at the end of the 60 minute study. More PDGF was released after a 5 minute saline wash, and washing in an elution buffer was somewhere in the middle between water and saline washes.

The washes were conducted as follows. The bone graft sample (−0.1 g) was placed in a small plastic tube and then either 1.0 ml of water, saline solution, or elution buffer was added to the sample. The mixture was allowed to sit at room temperature for 5 minutes with occasional gently mixing by hand. At the end of the 5 minutes, the fluid was pulled off the bone graft material using a pipet, and the remaining fluid was removed by compressing the material with a sterile cotton Q-tip applicator. Following this, a solution of 0.3 mg/ml rhPDGF-BB was added to the washed bone graft and samples were removed for quantification of PDGF by ELISA over 60 minutes.

### Exemplary Treatment Methods

In another aspect, the invention provides methods for treating an individual using any of the bone grafts and one or more of the polypeptides of interest described herein. In some embodiments, the method includes administering a bone graft and a polypeptide of interest (e.g., PDGF) to an individual. In some embodiments, the bone graft has been selected based on the level of protease activity. In some embodiments, at least a portion of the total amount of a protease associated with the bone graft has been removed from the bone graft prior to administering the bone graft to the individual. In some embodiments, the protease activity of two or more bone grafts is measured, and the bone graft with the lowest protease activity is administered to the individual in conjunction with the polypeptide of interest. In some embodiments, the protease activity of the selected bone graft is less than about 50 trypsin equivalents. In some embodiments, the protease activity of the selected bone graft is between about 50 to about 65 trypsin equivalents (such as about 50 to about 55, about 55 to about 60, or about 60 to about 65 trypsin equivalents). In some embodiments, the protease activity of the selected bone graft is about any of 50, 55, 60, or 65 trypsin equivalents. In some embodiments, the protease activity of the selected bone graft is less than about 50 trypsin equivalents (such as less than about 45, less that about 40, less than about 35, less that about 30, less than about 25, less than about 20, less than about 15, less than about 10, less than about 5, about 0 trypsin equivalents). In various examples, the activity of the polypeptide of interest (e.g. PDGF) may be maintained, after contact with the bone graft, for at least about 30 min, at least about 1 hour, at least about 2 hours, at least about 4 hours, at least about 8 hours, at least about 24 hours, at least about 2 days, at least about 3 days, at least about 5 days, at least about 1 week.

A mixture composed of bone graft (such as human bone allograft) and rhPDGF-BB can be used for the treatment of bones; promotion of the growth of bone, periodontium, ligament, or cartilage; bone augmentation; arthrodetic procedures; treatment of the vertebral column; treatment of osteonecrosis (ONJ) or osteoradionecrosis of the jaw (ORNJ); treatment of tendons or rotator cuff injuries; or distraction osteogenesis (see, for example, U.S. Pub. No. 2006/0084602, filed Jun. 23, 2005; U.S. Pub. No. 2007/0207185, filed Feb. 9, 2007; U.S. Pub. No. 2007/0129807, filed Nov. 17, 2006; PCT Pub. No. WO 2008/073628, filed Nov. 5, 2007; PCT App. No. WO PCT/US2008/065666, filed Jun. 3, 2008; PCT Pub. No. WO 2008/103690, filed Feb. 20, 2008; U.S. Pub. No. 2008/0027470, filed Jul. 2, 2008; U.S. App. No. 61/026,934, filed Feb. 7, 2008; which are each hereby incorporated by reference in their entireties, particularly with respect to treatment of bones; promotion of the growth of bone, periodontium, ligament, or cartilage; bone augmentation; arthrodetic procedures; treatment of the vertebral column; treatment of ONJ or ORNJ; treatment of tendons or rotator cuff injuries; or distraction osteogenesis). In addition to or as an alternative to PDGF, any other polypeptide of interest can be administered with a bone graft for treating, stabilizing, preventing, and/or delaying a bone, periodontium, ligament, cartilage, or tendon condition. Accordingly, the present invention also provides methods of treating bone (such as impaired or osteoporotic bone), fractures of the distal radius, vertebral bodies, periodontium, ligament, or cartilage. Additionally, the present invention provides methods of bone augmentation, arthrodetic procedures, treatment of osteonecrosis or osteoradionecrosis, treatment of tendons or rotator cuff injuries, and distraction osteogenesis.

In one embodiment, a method for treating bone comprises providing a composition comprising a polypeptide of interest and a bone graft (such as a polypeptide of interest disposed in a bone graft) and applying the composition to bone. In some embodiments, applying the composition to impaired bone can comprise molding the composition to the contours of the impaired bone. A composition, for example, can be molded into a bone fracture site thereby filling the volume created by the fracture. A method for treating bone, in another embodiment, comprises providing a composition comprising a polypeptide of interest and a bone graft, disposing the composition in a syringe, and injecting the composition at a site of impaired bone. In one embodiment, a composition comprising a polypeptide of interest and a bone graft can be injected into the volume created by a bone fracture. Injecting the composition, in some embodiments, can comprise penetrating tissue surrounding or covering a site of impaired bone with the syringe and depositing the composition at the site of impaired bone. In one embodiment, for example, a syringe can penetrate the skin and underlying tissue, such as muscle, covering a bone fracture site and subsequently deposit a composition of the present invention in and around the fracture. In such an embodiment, invasive techniques used to expose the fracture site for treatment, such as incisions and tissue removal, can be minimized.

In some embodiments, the composition is applied directly into a damaged site and the polypeptide of interest is released to facilitate bone healing. In one embodiment, the compositions of the present invention may be applied directly to impaired, damaged, injured, or fractured bone. In another embodiment, the compositions of the present invention may be applied to hardware used to facilitate fracture stabilization, for example, intramedullary nails, screws, and other hardware used by a physician of ordinary skill in the art, such as an orthopedic surgeon. In another embodiment, the compositions may be applied to openings in bone, such as sites of evulsion fractures, holes for screws, holes to receive intramedullary nails, or to the medullary canal.

The compositions of the present invention are used to facilitate healing of bone, including bone fractures, bone defects and bone fusions. Any bone may be treated with the compositions of the present invention, including but not limited to the humerus, ulna, radius, femur, tibia, fibula, patella, ankle bones, wrist bones, carpals, metacarpals, phalanges, tarsals, metatarsals, ribs, sternum, vertebrae, scapula, clavicle, pelvis, sacrum, and craniofacial bones. In some embodiments, the treated individual has osteoporosis.

The present invention also provides methods for the treatment of fractures, damage, or injury of the radius, particularly the distal radius and associated anatomical structures of the wrist. The present methods may accelerate the healing response in fractures of the distal radius, including bony union of the fracture site. Fractures of the distal radius, according to embodiments of the present invention, comprise all fracture types, including intra-articular and extra-articular fractures, as described by the AO classification system of distal radius fractures. In some embodiments, a distal radius fracture comprises a Type A fracture (extra-articular). In other embodiments, a distal radius fracture comprises a Type B fracture (partial articular). In another embodiment, a distal radius fracture comprises a Type C1 fracture (complete articular, simple articular, and metaphyseal fracture). In a further embodiment, a distal radius fracture comprises a Type C2 fracture (complete articular, simple articular with complex metaphyseal fracture). In some embodiments, a distal radius fracture comprises a Type C3 fracture (complete articular, complex articular, and metaphyseal fracture).

In another embodiment, a method for treating a fracture of the distal radius comprises providing a composition comprising a polypeptide of interest and a bone graft (such as a polypeptide of interest disposed in a bone graft) and applying the composition to a fracture in the distal radius. In some embodiments, applying the composition comprises injecting the composition into the fracture of the distal radius. In one embodiment, injecting comprises percutaneous injection of the composition into the fracture site. In another embodiment, the composition is injected into an open or surgically exposed fracture of the distal radius. In a further embodiment, applying comprises disposing the composition in the fracture with a spatula or other device. In some embodiments, a method for treating a fracture of the distal radius further comprises reducing the fracture and/or stabilizing the fracture. Reducing the fracture, according to some embodiments, comprises open reduction. In other embodiments, reducing the fracture comprises closed reduction. Moreover, stabilizing the distal radius fracture, in some embodiments, comprises applying an external or internal fixation device to the fracture, such as a volar plate. In another embodiment, a method for treating a fracture of the distal radius comprises accelerating new bone fill in the fracture, wherein accelerating comprises providing a composition comprising a polypeptide of interest and a bone graft and applying the composition to the fracture.

The present invention provides methods useful for treating structures of the vertebral column, including vertebral bodies. In some embodiments, methods are provided for promoting bone formation in a vertebral body. In other embodiments, methods are provided for preventing or decreasing the likelihood of vertebral compression fractures. In another embodiment, methods are provided for preventing or decreasing the likelihood of secondary vertebral compression fractures associated with vertebroplasty and kyphoplasty. The present methods are useful in treating vertebral bodies of individuals with osteoporosis. In some embodiments, the present invention provides methods for promoting bone formation in a vertebral body comprising providing a composition comprising a polypeptide of interest and a bone graft (such as a polypeptide of interest disposed in a bone graft) and applying the composition to at least one vertebral body. Applying the composition to at least one vertebral body, in some embodiments, comprises injecting the composition into the at least one vertebral body. In some embodiments, the composition can be applied to a plurality of vertebral bodies. Applying the composition, in some embodiments, comprises injecting at least one vertebral body with the composition. Compositions of the present invention, in some embodiments, are injected into the cancellous bone of a vertebral body. Vertebral bodies, in some embodiments, comprise thoracic vertebral bodies, lumbar vertebral bodies, or combinations thereof. Vertebral bodies, in some embodiments, comprise cervical vertebral bodies, coccygeal vertebral bodies, the sacrum, or combinations thereof.

In another aspect, the present invention provides methods comprising preventing or decreasing the likelihood of vertebral compression fractures, including secondary vertebral compression fractures. Preventing or decreasing the likelihood of vertebral compression fractures, according to embodiments of the present invention comprises providing a composition comprising a polypeptide of interest in a bone graft and applying the composition to at least one vertebral body. In some embodiments, applying the composition to at least one vertebral body comprises injecting the composition into the at least one vertebral body. In one embodiment, the composition is applied to a second vertebral body, in some instances an adjacent vertebral body, subsequent to a vertebroplasty or kyphoplasty of a first vertebral body. In some embodiments, a composition comprising a polypeptide of interest disposed in a bone graft is applied to at least one high risk vertebral body. “High risk vertebral bodies” (HVB), as used herein, refer to vertebral bodies of vertebrae T5 through T12 as well as L1 through L4, which are at the greatest risk of undergoing secondary vertebral compression fracture.

In some embodiments, a composition of the present invention is applied to a second vertebral body subsequent to vertebroplasty or kyphoplasty of a first vertebral body. In some embodiments, the second vertebral body is adjacent to the first vertebral body. In other embodiments, the second vertebral body is not adjacent to the first vertebral body. In a further embodiment, a composition of the present invention is applied to a third vertebral body subsequent to vertebroplasty or kyphoplasty of a first vertebral body. In some embodiments, the third vertebral body is adjacent to the first vertebral body. In other embodiments, the third vertebral body is not adjacent to the first vertebral body. Embodiments of the present invention additionally contemplate application of compositions provided herein to a plurality of vertebral bodies, including high risk vertebral bodies, subsequent to vertebroplasty or kyphoplasty of a first vertebral body. It is to be understood that first, second, and third vertebral bodies, as used herein, do not refer to any specific position in the vertebral column as methods for inhibiting vertebral compression fractures, including secondary compression fractures, can be applied to all types of vertebral bodies including thoracic vertebral bodies, lumbar vertebral bodies, cervical vertebral bodies, coccygeal vertebral bodies, and the sacrum.

The invention also provides methods for promoting growth of bone, periodontium, ligament, or cartilage in a mammal by applying to the bone, periodontium, ligament, or cartilage a composition comprising a polypeptide of interest and a bone graft (such as a polypeptide of interest disposed in a bone graft). In some embodiments, the method includes the healing of bone, periodontium, ligament, or cartilage, and/or the regeneration of such tissues and structures. In some embodiments, the bone, periodontium, ligament, or cartilage is damaged or wounded and requires regeneration or healing.

The present invention also provides methods of performing bone augmentation procedures. In one embodiment, a method of performing a bone augmentation procedure comprises providing a composition comprising a polypeptide of interest and a bone graft (such as a polypeptide of interest disposed in a bone graft), and applying the composition to at least one site of desired bone augmentation. In some embodiments, a method of performing a bone augmentation procedure comprises applying the composition to at least one site of bone augmentation in the maxilla or mandible. In some embodiments, the composition is packed into a site of desired bone augmentation in the maxilla or mandible. In another embodiment, the polypeptide of interest is applied to the implantation site before, and optionally after placement of the composition comprising the polypeptide of interest and the bone graft into the implantation site. By enhancing the deposition of bone in the maxilla or mandible, the alveolar ridge may be enhanced so as to subsequently receive an implant. Such implants may be used for a variety of purposes, including as a support for a tooth or other dental device, and for various oral and maxillofacial applications, including extraction sockets, sinus elevation, and ridge augmentation.

The present invention also provides methods of performing arthrodetic procedures. In one embodiment, a method of performing an arthrodetic procedure comprises providing a composition comprising a polypeptide of interest and a bone graft (such as a polypeptide of interest disposed in a bone graft) and applying the composition to a site of desired bone fusion in a joint. In some embodiments, a method of performing an arthrodetic procedure comprises applying the composition to a site of desired bone fusion in a plurality of joints. In some embodiments, the composition is packed into a site of desired bone fusion in a joint. In some embodiments, the composition can be packed such that the composition is in contact with the entire surface area of the bones to be fused in the joint. The composition may additionally be applied to the vicinity of the bone fusion site to further strengthen the fused joint. In some embodiments, a method of performing an arthrodetic procedure further comprises aligning the joint and inserting at least one fixation device, such as a screw, into at least one bone of the joint. In some embodiments, a plurality of screws are inserted into at least one bone of the joint. In another embodiment, a method of the present invention comprises accelerating bony union in an arthrodetic procedure wherein accelerating bony union comprises providing a composition comprising a polypeptide of interest and a bone graft (such as a polypeptide of interest disposed in a bone graft) and applying the composition to at least one site of bone fusion in a joint.

Bones in any joint may be fused using the compositions and methods of the present invention. Such joints include, but are not limited to joints of the foot, toes, ankle, knee, hip, spine, rib, sternum, clavicle, joint, shoulder, scapula, elbow, wrist, hand, fingers, jaw and skull. It is to be understood that the present invention may apply to any desired site for arthrodesis in the appendicular or spinal skeleton. In one embodiment of the present invention, arthrodetic procedures comprise arthrodesis of the foot and ankle including subtalar arthrodesis, talonavicular arthrodesis, triple arthrodesis, and ankle arthrodesis.

The invention also provides methods for treating, preventing, or slowing the progression of ONJ or ORNJ. The compositions may be administered through any appropriate means. In one embodiment, administration of the composition comprising a polypeptide of interest and a bone graft (such as a polypeptide of interest disposed in a bone graft) may occur through direct application of the composition at the desired site. In another embodiment, administration of the composition comprising a polypeptide of interest and a bone graft (such as a polypeptide of interest disposed in a bone graft) may occur through direct application of the composition at the desired site. Such sites include, but are not limited to, the maxilla, the mandible and their adnexia which includes the alveolar structures, and any other bone or soft tissues affected by ONJ or ORNJ. In the mandible, sites anterior to the retromolar pad may constitute a desired site. For example, when a surgical field is open in the maxilla or mandible of a patient with ONJ or ORNJ, and a necrotic site is debrided and prepared, the composition may be applied through a syringe delivery, through a needle or cannula, by direct application with a spatula, forceps, spoon or other acceptable means. In other embodiments, when a site predicted to be vulnerable to ONJ or ORNJ is identified, the site may be exposed surgically and the composition applied, or the composition may be applied by syringe and needle injection through the skin to the vicinity of the desired site without surgically exposing the site in the mandible or maxilla. In other embodiments, the composition may be applied to the desired site through direct percutaneous administration.

In some embodiments, the composition is administered concurrently with the dental procedure or shortly after the dental procedure. For example, a patient at risk and having a dental surgical procedure such as an extraction has the polypeptide-containing composition, in one embodiment, co-administered with, for example, a dental extraction medicament or dressing. Yet another example is an oro-dental cystectomy where the polypeptide-containing composition is placed into the cystic cavity. Yet another example includes a periodontal procedure where gingival tissues were incised and alveolar and/or inter-radicular osseo-dental surgery were performed and the polypeptide-containing composition is co administered with the periodontal therapy dressing.

In some embodiments, the quantity of the composition administered is determined by the bone volume that had been surgically removed, for example from an extraction socket, a cystrectomy, or during periodontal bone surgery. In some embodiments, for the prophylactic treatment of ORNJ or ONJ with a composition, radiographic determination of a thickening of the periodontal ligament may be considered a diagnostic criterion.

The present invention also provides methods for the attachment or reattachment of tendons to bone, the strengthening of tendon attachment to bone as well as the treatment of tendons, such as tendons exhibiting tearing, delamination, or any other strain or deformation. In one embodiment, a method for reattaching a tendon to bone comprises providing a composition comprising a PDGF solution disposed in a biocompatible matrix and applying the composition to at least one site of tendon reattachment on the bone. In another embodiment, a method of strengthening the attachment of a tendon to a bone comprises providing a composition comprising a PDGF solution disposed in a biocompatible matrix and applying the composition to at least one site of tendon attachment to bone. Methods of strengthening tendon attachment to bone, in some embodiments, assist in preventing or inhibiting tendon detachment from bone, such as in rotator cuff injuries.

The present invention also provides methods of treating rotator cuff tears. In one embodiment, a method for treating rotator cuff tears comprises providing a composition comprising a polypeptide of interest and a bone graft (such as a polypeptide of interest disposed in a bone graft) and applying the composition to at least one site of tendon reattachment on the humeral head. In some embodiments, applying the composition to at least one site of tendon reattachment can comprise molding the composition to the contours of the reattachment site on the humeral head. A composition, for example, can be molded into a channel formed on a surface of the humeral head for receiving the detached tendon. The composition may be applied to the vicinity of the insertion site of the tendon into bone to further strengthen the attachment.

In some embodiments, a method for treating rotator cuff tears further comprises disposing at least one anchoring means, such as a bone anchor in the humeral head, wherein the bone anchor further comprises a polypeptide of interest (such as a polypeptide of interest disposed in a bone graft), and coupling at least one detached tendon to the bone anchor. In embodiments of the present invention, tendons can be secured to bone anchors through sutures. Sutures may also be soaked in solutions of a polypeptide of interest (such as PDGF) or coated in polypeptide-compositions before use.

In another embodiment, a method of treating a tendon comprises providing a composition comprising a polypeptide of interest and a bone graft (such as a polypeptide of interest disposed in a bone graft) and applying the composition to a surface of at least one tendon. In some embodiments, the at least one tendon is an injured or damaged tendon, such as tendon exhibiting tearing, delamination, or any other deformation.

In some embodiments, a method for stimulating and/or accelerating osteogenesis comprises providing a composition comprising a polypeptide of interest and a bone graft (such as a polypeptide of interest disposed in a bone graft) and applying an effective amount of the composition to at least one site of bone distraction. In some embodiments, the composition comprising a polypeptide of interest and a bone graft (such as a polypeptide of interest disposed in a bone graft) is applied during bone distraction. In other embodiments, the composition is applied after bone distraction. In one embodiment, an effective amount of the composition is applied during and after bone distraction.

The present invention also provides methods of accelerating bone union following bone distraction. In some embodiments, a method for accelerating bone union following bone istraction comprises providing a composition comprising a polypeptide of interest and a bone graft (such as a polypeptide of interest disposed in a bone graft) and applying an effective amount of the composition to at least one site of bone distraction.

The present invention additionally provides methods of performing osteodistraction procedures. In one embodiment, a method of performing an osteodistraction procedure comprises (a) partitioning a bone into a first bone segment and a second bone segment, (b) moving at least one of the first and second bone segments to produce a space between the first and second bone segments, and (c) stimulating osteogenesis in the space, wherein stimulating osteogenesis comprises providing a composition a polypeptide of interest and a bone graft (such as a polypeptide of interest disposed in a bone graft) and at least partially disposing an effective amount of the composition in the space. In some embodiments, steps (b) and (c) can be repeated as many times as necessary to lengthen the bone any desired amount.

In some embodiments of methods of the present invention, applying the composition comprises injecting the composition in a site of bone distraction. In one embodiment, injecting comprises percutaneous injection of the composition in the distraction site. In another embodiment, the composition is injected into an open or surgically exposed site of bone distraction. In a further embodiment, applying the composition comprises disposing the composition in a site of bone distraction with a spatula or other device.

In some embodiments, a composition of the present invention is applied to at least one site of bone distraction during the distraction phase of an osteodistraction procedure. In other embodiments, a composition of the present invention is applied to at least one site of bone distraction during the consolidation phase following bone distraction. In a further embodiment, a composition of the present invention is applied to at least one site of bone distraction during the distraction and consolidation phases.

As provided herein, osteodistraction procedures, according to embodiments of the present invention, comprise those used in the treatment of bilateral mandibular hypoplasia, hemifacial microsomia, congenital short femur, fibular hemimelia, hemiatrophy, achondroplasia, neurofibromatosis, bow legs, growth plate fractures, bone defects, craniofacial applications, osteomyelitis, septic arthritis, and poliomyelitis.

In some embodiments, the bone graft is screened using standard methods to make sure it is not contaminated with a virus from the donor. The type of bone to be treated may be the same as, or different from, the type of bone that is used as the source of the bone graft.

The methods of the present invention can be used to treat any individual. For use herein, unless clearly indicated otherwise, “an individual” as used herein intends a mammal, including but not limited to, a primate (e.g., a human, monkey, gorilla, ape, lemur, etc.), a bovine, an equine, a porcine, an ovine, a canine, and a feline. Thus, the invention finds use in both human medicine and in the veterinary context, including use in agricultural animals and domestic pets. The individual may have been diagnosed with, is suspected of having, or is at risk of developing an indication, such as a bone, periodontium, ligament, cartilage, or tendon condition. The individual may exhibit one or more symptoms associated with the indication. The individual can be genetically or otherwise predisposed to developing such a condition.

As used herein, “in need thereof” includes individuals who have a condition or disease or are “at risk” for the condition or disease. As used herein, an “at risk” individual is an individual who is at risk of development of a condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). These risk factors include, but are not limited to, age, sex, race, diet, history of previous disease, presence of precursor disease, genetic (i.e., hereditary) considerations, and environmental exposure.

As used herein, “treatment” or “treating” is an approach for obtaining beneficial or desired results, including desirably clinical results. For purposes of this invention, beneficial or desired clinical results include, but are not limited to, one or more of the following: decreasing symptoms resulting from the disease, increasing the quality of life of those suffering from the disease, decreasing the dose of other medications required to treat the disease, and/or delaying the progression of the disease.

As used herein, “delaying development of a disease” means to defer, hinder, slow, retard, stabilize, and/or postpone development of the disease (such as a bone, periodontium, ligament, cartilage, or tendon condition). This delay can be of varying lengths of time, depending on the history of the disease and/or individual being treated. As is evident to one skilled in the art, a sufficient or significant delay can, in effect, encompass prevention, in that the individual does not develop the disease.

As used herein, an “effective dosage” or “effective amount” of bone graft, polypeptide, drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective dosage can be administered in one or more administrations. For purposes of this invention, an effective dosage of bone graft, polypeptide, drug compound, or pharmaceutical composition is an amount sufficient to accomplish prophylactic or therapeutic treatment either directly or indirectly. As is understood in the clinical context, an effective dosage of a bone graft, polypeptide, drug, compound, or pharmaceutical composition may or may not be achieved in conjunction with another bone graft, polypeptide, drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality (such as a bone graft) in addition to another treatment modality (such as a polypeptide of interest). As such, “in conjunction with” refers to administration of one treatment modality before, during, or after administration of the other treatment modality to the individual. In some embodiments, a bone graft and a polypeptide of interest are administered simultaneously, sequentially, or concurrently. In some embodiments, the polypeptide of interest binds to or becomes disposed in the bone graft before both the polypeptide of interest and bone graft are simultaneously administered to an individual. In some embodiments, the bone graft is administered to the individual (with or without bound polypeptide of interest), and then the polypeptide of interest is administered at or near the site of the bone graft in the individual.

In some embodiments, solutions comprising the polypeptide of interest are formed by solubilizing the polypeptide of interest in one or more buffers. Buffers suitable for use in the polypeptide solutions of the present invention can comprise, but are not limited to, carbonates, phosphates (e.g., phosphate-buffered saline), histidine, acetates (e.g., sodium acetate), acidic buffers such as acetic acid and HCl, and organic buffers such as lysine, Tris buffers (e.g., tris(hydroxymethyl)aminoethane), N-2-hydroxyethylpiperazine-N′-2-ethanesulfonic acid (HEPES), and 3-(N-morpholino) propanesulfonic acid (MOPS). Buffers can be selected based on biocompatibility with the polypeptide of interest and the buffer's ability to impede undesirable polypeptide modification. Buffers can additionally be selected based on compatibility with host tissues. In one embodiment, sodium acetate buffer is used. The buffers may be employed at different molarities, for example about 0.1 mM to about 100 mM, about 1 mM to about 50 mM, about 5 mM to about 40 mM, about 10 mM to about 30 mM, about 15 mM to about 25 mM, or any molarity within these ranges. In some embodiments, an acetate buffer is employed at a molarity of about 20 mM. In another embodiment, solutions comprising the polypeptide of interest may be formed by solubilizing lyophilized the polypeptide of interest in water, wherein prior to solubilization the polypeptide of interest is lyophilized from an appropriate buffer.

Compositions and methods provided by the present invention may comprise a bone graft and a solution of the polypeptide of interest, wherein the solution is dispersed in the bone graft. In some embodiments, the polypeptide of interest (such as PDGF) is present in the solution in a concentration ranging from about 0.01 mg/ml to about 10.0 mg/ml, from about 0.05 mg/ml to about 5.0 mg/ml, or from about 0.1 mg/ml to about 1.0 mg/ml. In some preferred embodiments, the polypeptide of interest is present in the solution at a concentration of 0.3 mg/ml. In other embodiments, the polypeptide of interest is present in the solution at any one of the following concentrations: about 0.05 mg/ml, about 0.1 mg/ml, about 0.15 mg/ml, about 0.2 mg/ml, about 0.25 mg/ml, about 0.3 mg/ml, about 0.35 mg/ml, about 0.4 mg/ml, about 0.45 mg/ml, about 0.5 mg/ml, about 0.55 mg/ml, about 0.6 mg/ml, about 0.65 mg/ml, about 0.7 mg/ml, about 0.75 mg/ml, about 0.8 mg/ml, about 0.85 mg/ml, about 0.9 mg/ml, about 0.95 mg/ml, or about 1.0 mg/ml. It is to be understood that these concentrations are simply examples of particular embodiments, and that the concentration of the polypeptide of interest may be within any of the concentration ranges stated above or at any other suitable concentration. Various amounts of the polypeptide of interest may be used in the compositions of the present invention. Amounts of the polypeptide of interest that could be used include amounts in the following ranges: about 1 μg to about 50 mg, about 10 μg to about 25 mg, about 100 μg to about 10 mg, and about 250 μg to about 5 mg.

In some embodiments, about 1.5 mL of a solution of a polypeptide of interest (such as PDGF or another growth factor) is combined with about 2 cubic centimeters (cc) of bone graft. In various embodiments, the ratio of the amount of solution of a polypeptide of interest (such as PDGF or another growth factor) to bone graft is about 1:2, 3:4, or 1:1 (ratio of liquid volume (mL) to dry volume (cc)). In some embodiments, the concentration of PDGF in the solution is between about 0.1 to about 1.0 mg/mL.

The concentration of the polypeptide of interest (such as PDGF or other growth factors) in embodiments of the present invention can be determined by using an enzyme-linked immunoassay as described in U.S. Pat. Nos. 6,221,625; 5,747,273; and 5,290,708 (which are each hereby incorporated by reference in their entireties, particularly with respect to ELISA assays), or any other assay known in the art for determining polypeptide concentration. When provided herein, the molar concentration of PDGF is determined based on the molecular weight of PDGF dimer (e.g., PDGF-BB, MW about 25 kDa).

Solutions comprising the polypeptide of interest (such as PDGF), according to embodiments of the present invention, can have a pH ranging from about 3.0 to about 8.0. In one embodiment, a solution comprising the polypeptide of interest has a pH ranging from about 5.0 to about 8.0, more desirably about 5.5 to about 7.0, most desirably about 5.5 to about 6.5, or any value within these ranges. The pH of solutions comprising the polypeptide of interest, in some embodiments, can be compatible with the prolonged stability and efficacy of the polypeptide of interest or any other desired biologically active agent. For example, PDGF is generally more stable in an acidic environment. Therefore, in accordance with some embodiments, the present invention comprises an acidic storage formulation of the polypeptide solution (such as a PDGF solution). In accordance with some embodiments, the solution desirably has a pH from about 3.0 to about 7.0, and more desirably from about 4.0 to about 6.5. The biological activity of the polypeptide of interest, however, can be optimized in a solution having a neutral pH range. Therefore, in other embodiments, the present invention comprises a neutral pH formulation of the polypeptide solution. In accordance with this embodiment, the polypeptide solution desirably has a pH from about 5.0 to about 8.0, more desirably about 5.5 to about 7.0, most desirably about 5.5 to about 6.5.

In some embodiments, the pH of the polypeptide containing solution may be altered to optimize the binding kinetics of the polypeptide of interest to a matrix substrate. If desired, as the pH of the material equilibrates to adjacent material, the bound the polypeptide of interest may become labile. The pH of solutions comprising the polypeptide of interest, in some embodiments, can be controlled by the buffers recited herein. Various polypeptides demonstrate different pH ranges in which they are stable. Polypeptide stabilities are primarily reflected by isoelectric points and charges on the polypeptides. The pH range can affect the conformational structure of a polypeptide and the susceptibility of a polypeptide to proteolytic degradation, hydrolysis, oxidation, and other processes that can result in modification to the structure and/or biological activity of the polypeptide.

Compositions and methods of the present invention, according to some embodiments, can further comprise one or more biologically active agents in addition to the polypeptide of interest. Exemplary biologically active agents include organic molecules, inorganic materials, polypeptides, peptides, nucleic acids (e.g., genes, gene fragments, small-interfering ribonucleic acids (siRNAs), gene regulatory sequences, nuclear transcriptional factors and antisense molecules), nucleoproteins, polysaccharides (e.g., heparin), glycoproteins, and lipoproteins. Non-limiting examples of biologically active compounds that can be incorporated into compositions of the present invention, including, e.g., anti-cancer agents, antibiotics, analgesics, anti-inflammatory agents, immunosuppressants, enzyme inhibitors, antihistamines, hormones, muscle relaxants, prostaglandins, trophic factors, osteoinductive polypeptides, growth factors, vitamins (such as vitamin D₃), calcium supplements, osteoclast inhibitors (such as bisphosphonates), and vaccines, are disclosed in U.S. Pub. No. 2006/0084602, filed Jun. 23, 2005 (which is hereby incorporated by reference in its entirety, particularly with respect to biologically active agents). In other embodiments, the compositions and methods of the preset invention can further comprise cell culture media, stabilizing polypeptides such as albumin, antibacterial agents, protease inhibitors (e.g., ethylenediaminetetraacetic acid (EDTA), ethylene glycol-bis(beta-aminoethylether)-N,N,N′,N′-tetraacetic acid (EGTA), aprotinin, E-aminocaproic acid (EACA), etc.), peptide or organic molecules containing protease inhibitors (such as alpha 1 antitrypsin (trypsin/elastase inhibitor), ovomucoid, pancreatic inhibitor, amastin-HCl (metalloprotease inhibitors), antipain (serine and cysteine protease inhibitor), aprotinin (serine protease inhibitor)), and/or other growth factors such as fibroblast growth factors (FGFs), epidermal growth factors (EGFs), transforming growth factors (TGFs), keratinocyte growth factors (KGFs), insulin-like growth factors (IGFs), hemostasis factors such as FXIII, bone morphogenetic polypeptides (BMPs), or other PDGFs including compositions of PDGF-AA, PDGF-BB, PDGF-AB, PDGF-CC and/or PDGF-DD. In some embodiments, the compositions and methods of the invention further comprise a stabilizing agent, such as a polypeptide that is cleaved by one or more proteases associated with the bone graft. In some embodiments, one or more polypeptides that are cleaved by a protease associated with the bone graft are added to the bone graft to reduce the amount of the polypeptide of interest (such as PDGF) that is cleaved by the protease.

In some embodiments, the compositions and methods of the invention further comprise at least one contrast agent. In one embodiment, contrast agents are optionally combined with the compositions of the present invention in order to facilitate visualization of the applied or injected composition. Contrast agents, according to embodiments of the present invention, are substances operable to at least partially provide differentiation of two or more bodily tissues when imaged. Contrast agents, according to some embodiments, comprise cationic contrast agents, anionic contrast agents, nonionic contrast agents, or mixtures thereof. In some embodiments, contrast agents comprise radiopaque contrast agents. Radiopaque contrast agents, in some embodiments, comprise iodo-compounds including (S-N,N′-bis[2-hydroxy-1-(hydroxymethyl)-ethyl]-2,4,6-triiodo-5-lactamid-oisophthalamide (Iopamidol) and derivatives thereof (see, for example, U.S. Pub. No. 2007/0207185, filed Feb. 9, 2007, which is hereby incorporated by reference in its entirety, particularly with respect to contrast agents).

In some embodiments, at least one agent (such as a biologically active agent, protease inhibitor, or contrast agent) is administered locally. In such embodiments, the agent can be incorporated into the bone graft or otherwise disposed in and around a site of a bone to be treated. In other embodiments, at least one agent (such as a biologically active agent) is administered systemically, orally, or intravenously to an individual. In various embodiments, one or more protease inhibitors are added to the bone graft, before, during, or after the addition of the polypeptide of interest to the bone graft.

### Exemplary Kits

In another aspect, the present invention provides kits comprising a first container comprising a polypeptide of interest described herein (such as PDGF) and a second container comprising a bone graft described herein (such as a human bone graft). In some embodiments, the first container has a solution that comprises a predetermined concentration of the polypeptide of interest (such as PDGF). The concentration of polypeptide of interest, in some embodiments, can be predetermined according to the nature of the bone, periodontium, ligament, cartilage, or tendon condition being treated. In some embodiments, the bone graft comprises a predetermined amount according to the nature of the bone, periodontium, ligament, cartilage, or tendon condition being treated. In some embodiments, there may be more than one container comprising different types of bone grafts. In some embodiments, the protease activity of the selected bone graft is less than about 50 trypsin equivalents. In some embodiments, the protease activity of the selected bone graft is between about 50 to about 65 trypsin equivalents (such as about 50 to about 55, about 55 to about 60, or about 60 to about 65 trypsin equivalents). In some embodiments, the protease activity of the selected bone graft is about any of 50, 55, 60, or 65 trypsin equivalents. In some embodiments, the protease activity of the selected bone graft is less than about 50 trypsin equivalents (such as less than about 45, less that about 40, less than about 35, less that about 30, less than about 25, less than about 20, less than about 15, less than about 10, less than about 5, about 0 trypsin equivalents). A syringe, in some embodiments, can facilitate dispersion of a solution of the polypeptide of interest in the bone graft for application at a surgical site, such as a site of bone damage or injury.

The kit may also contain instructions for use. The instructions relating to the use of a bone graft and polypeptide of interest to treat a bone, periodontium, ligament, cartilage, or tendon condition generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert indicates that the composition is used for treating, preventing, or delaying development of a bone, periodontium, ligament, cartilage, or tendon condition described herein. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. A kit or container may have a sterile access port (for example the container may be an intravenous solution bag or a vial having a stopper pierceable by a hypodermic injection needle).

The kit may further comprise a biologically active agent (in addition to the polypeptide of interest), a contrast agent, a protease inhibitor, buffer, or any combination of the foregoing.

The following Examples are provided to illustrate but not limit the invention.

### EXAMPLES

### Example 1

### Binding and Release of PDGF from a Freeze-Dried Bone Graft (“FDBA”) Matrix

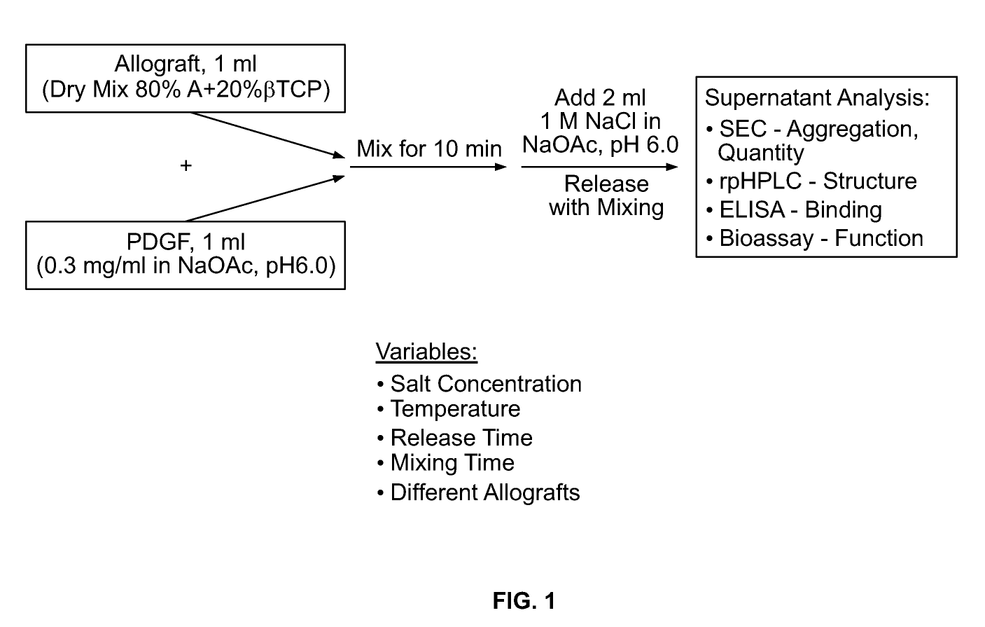

FIG. 1 summarizes an exemplary protocol for studying the binding and release of PDGF from bone graft. For some experiments, the amounts were scaled down two or ten times to preserve the bone graft materials. In particular, 100 μl of human bone graft (LifeNet, Inc.) was mixed with 100 μl of PDGF at a concentration of 0.3 mg/mL in 20 mM NaOAc, pH 6.0 for ten minutes. The bone graft consisted of dry particles. The bone graft was freeze-dried before being sold (referred to as freeze-dried bone allograft, or FDBA). Then, 200 μl of NaCl (at various concentrations) in 20 mM NaOAc, pH 6.0 or phosphate buffered saline (PBS) (without NaOAc) was added. Different concentrations of NaCl were used to give the final concentrations of NaCl listed in FIG. 2. The solution was then incubated for either 1 hour or 24 hours. PDGF in solution was separated from insoluble human bone graft by centrifugation at ˜2000×g for 5 minutes at 4° C., and then the amount of rhPDGF-BB released from the matrix was determined by colorimetric ELISA Quantikine assy (R & D Systems, Minneapolis, Minn.) using plates coated with the receptor to human PDGF-BB. FIG. 2 indicates that a significant amount of PDGF was released by the salt incubation and that the release is both fast and salt dependent.

FIG. 3 indicates that the PDGF release is independent of the mixing time and that the recovery was 40-60%. In particular, PDGF (0.5 ml of 0.3 mg/ml) was incubated with the freeze-dried bone graft (˜0.5 ml) and mixed for 10 minutes, 60 minutes, or overnight at room temperature. Then 1 mL of 1 M NaCl was added as described above for FIG. 1. The final NaCl concentration was 667 mM. PDGF was separated from the bone graft by centrifugation at 15,334×g for 2 minutes and the supernatant containing PDGF was analyzed by SEC, RPHPLC, ELISA, and non-reducing SDS-PAGE. The PDGF in a control experiment was normalized as 100%. rhPDGF-BB eluted from human FDBA was electrophoretically indistinguishable from a control sample of rhPDGF-BB by non-reducing SDS-PAGE electrophoresis (data not shown). Furthermore, rhPDGF eluted from FDBA with NaCl after ten minutes retained its biopotency compared to a control sample of rhPDGF-BB as determined with a standard MG-63 cell-based bioassay (data not shown). Released PDGF was measured using a standard ELISA assay. The ELISA assay was performed using Quantikine® Human PDGF-BB Immunoassay from R&D Systems, Inc. according to the manufacturer's protocol (see, for example, world wide web at rndsystems.com/pdf/dbb00.pdf). Briefly, a receptor for human PDGF-BB was coated onto a plate, then PDGF was bound and evaluated with a secondary antibody-HRP conjugate and the chromogen tetramethylbenzidine for detection. FIG. 3 suggests that some PDGF remains bound to the bone graft. Alternatively, the concentrations of the rhPDGF-BB in supernatants were determined using the size exclusion HPLC (SEC) on TOSOH Biosep TSK-Gel, 7.8 mm×3 cm HPLC column and TOSOH Biosep TSK-Gel, 6.0 mm×4.0 cm precolumn (Tosoh Bioscience, San Francisco, Calif.) by integration of the elution profile of PDGF and using calibration at 5 different concentrations of standard PDGF as shown in FIG. 4C.

SEC was used to quantify the amount of PDGF eluted from the freeze-dried bone graft matrix and to analyze its native structure and aggregation (FIGS. 4A-4C, 5A, 5B, 6A, and 6B). Bone graft (˜1 ml) was mixed with PDGF (0.3 mg/ml; 1:1 vol/vol), eluted instantly with 2 ml of 1 M NaCl, and centrifuged at 15,334×g for 2 minutes. The 100 ul of supernatants were analyzed by SEC. SEC was performed using a TOSOH BiosepTSK-Gel, 7.8 mm×30 cm HPLC column with TOSOH Biosep TSK-Gel 6.0 mm×4.0 cm HPLC guard column (TOSOH Bioscience, S. San Francisco, Calif.) with the mobile phase 0.4 M NaCl in 0.05 M sodium acetate, pH 4.0 at a flow rate of 0.8 ml/min at room temperature. FIG. 4C is a graph showing the calibration of the SEC column.

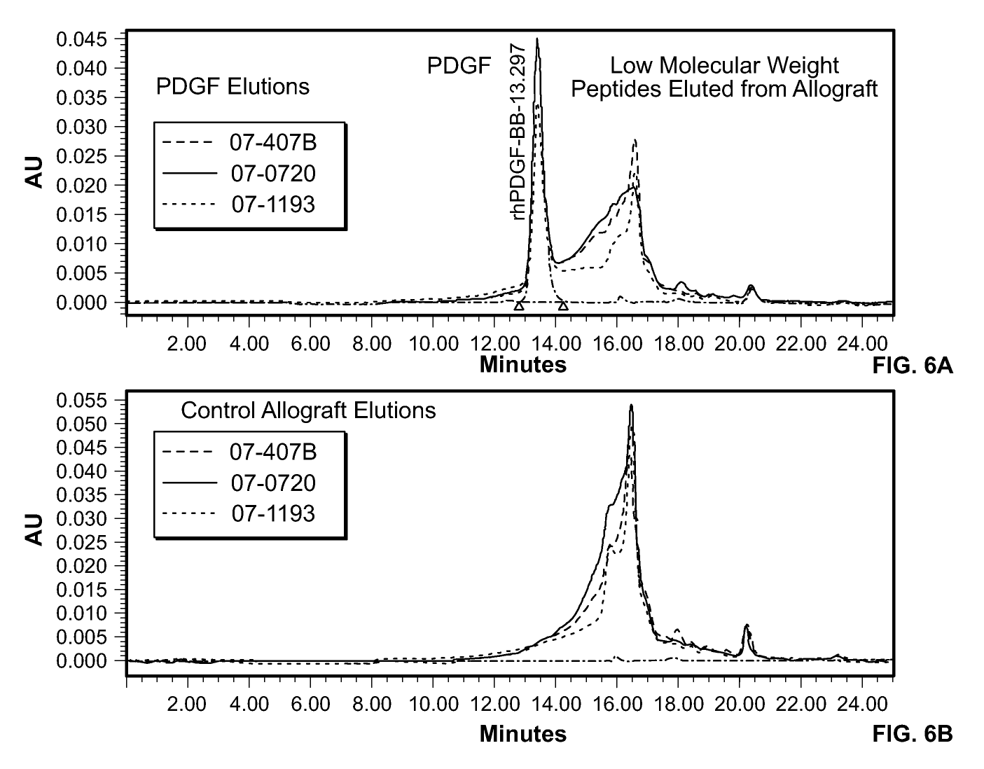

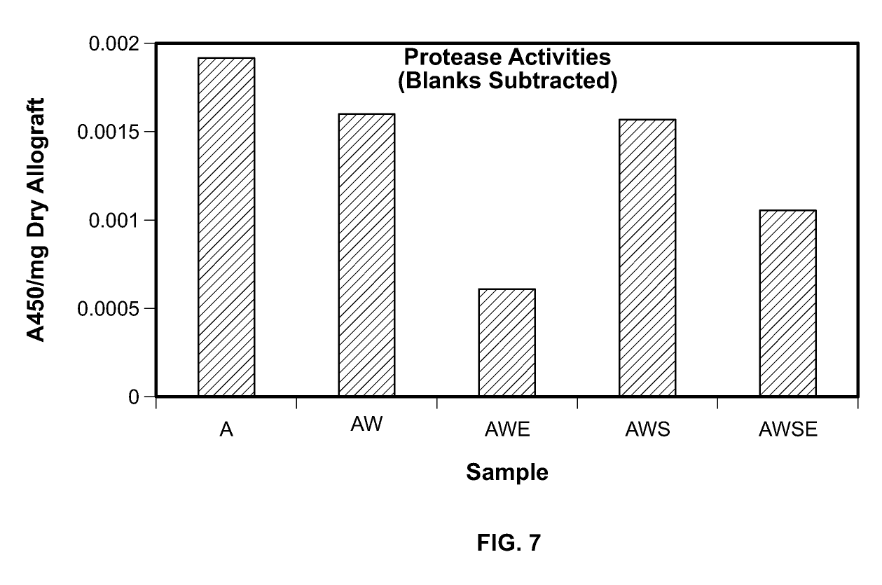

SEC shows the native size of PDGF and its interactions, PDGF aggregation, and other components of the sample (FIGS. 4A and 4B). For example, if a new high molecular weight peak appears in the SEC profile of bone graft and PDGF compared to the bone graft control or the PDGF control, then either interaction of bone graft with PDGF occurred and/or PDGF itself aggregated into conglomerates of dimers due to its interaction with the bone graft. A new high molecular weight peak was observed under certain conditions. The SEC profile was temperature and release time dependent (FIGS. 5A and 5B). These chromatograms show significant elution of non-specific polypeptides at higher temperatures and longer release times. Elution of PDGF remains approximately the same under the conditions tested for FIGS. 5A and 5B. The SEC profile was also sample dependent (FIGS. 6A and 6B).

Other compounds, such as MEM and acetic acid, were tested in place of NaCl and also resulted in the release of some PDGF from the freeze-dried bone graft.

### Example 2

### Binding and Release of PDGF from a Bone Graft

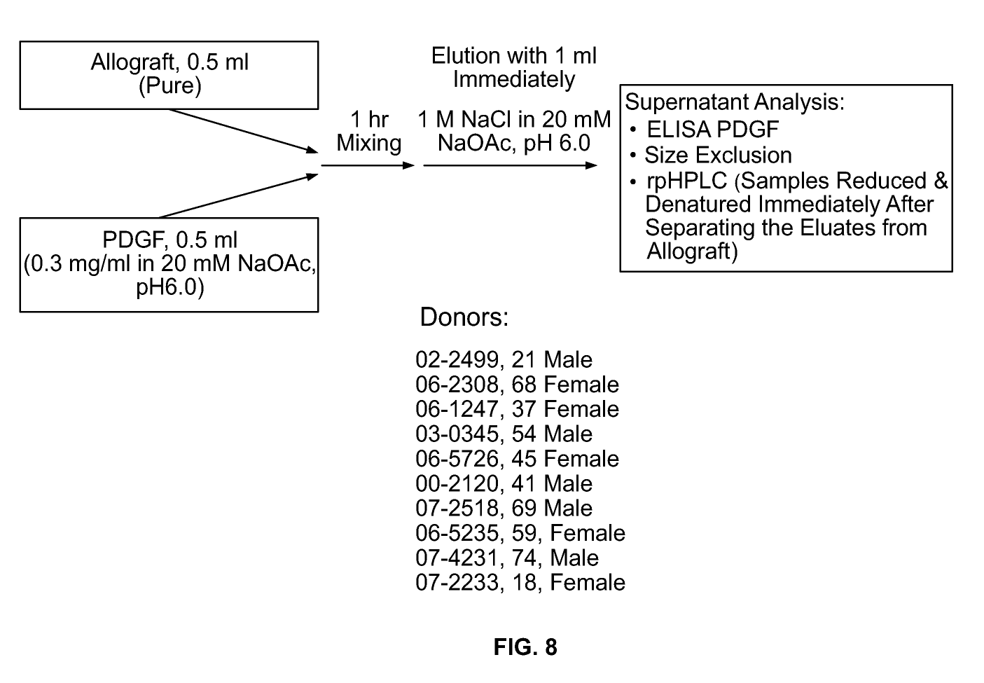

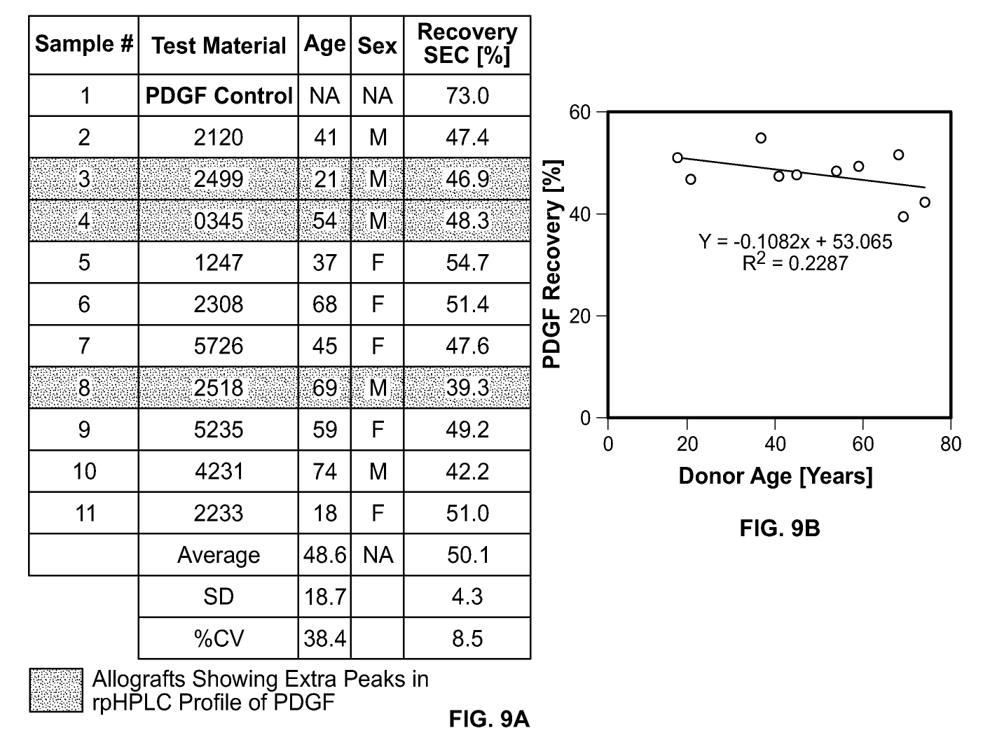

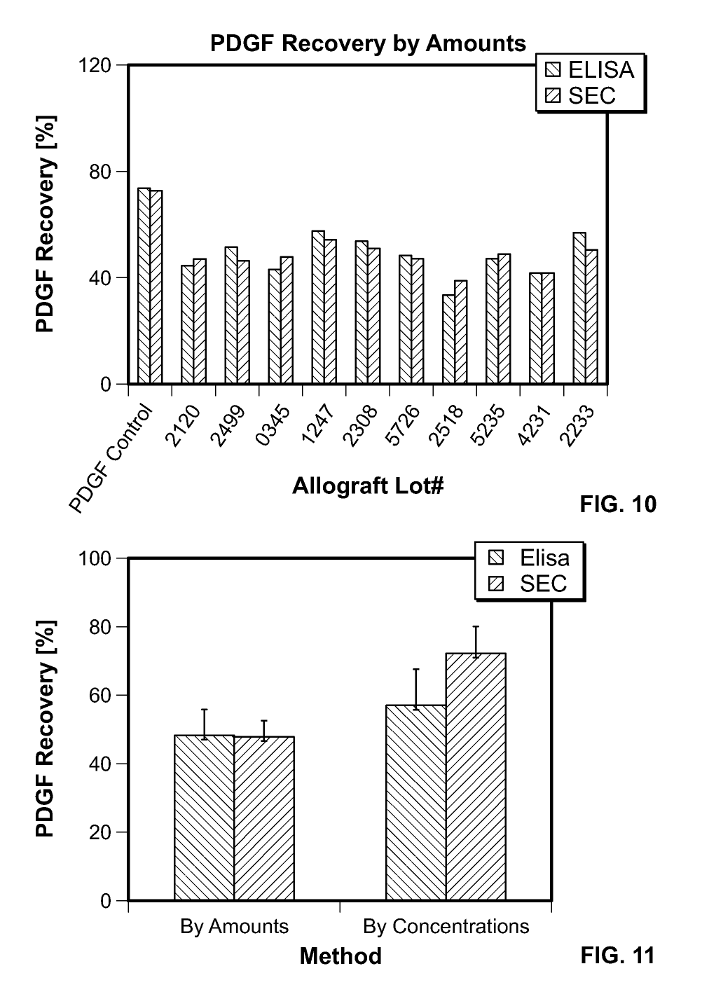

FIG. 8 summarizes an exemplary protocol for studying the binding and release of PDGF from bone graft for different lots of bone graft. Specifically, 0.5 ml of human bone graft was mixed with 0.5 ml of PDGF at a concentration of 0.3 mg/mL in 20 mM NaOAc, pH 6.0 for one hour. Then, 1 mL of 1M NaCl in 20 mM NaOAc, pH 6.0 was added. After adding the salt, the supernatant was immediately separated from the bone graft by centrifugation at 15,334×g and subjected to further analysis (SEC, RPHPLC, and ELISA). There was no statistically significant age/gender effect of human bone graft on PDGF release (FIGS. 9A and 9B). The PDGF was quantitated for FIGS. 9A and 9B based on total peak area using SEC and RPHPLC. FIG. 10 shows the recovery of PDGF from bone grafts measured by ELISA or SEC as described in Example 1 for various bone graft samples. The original amount of PDGF used in the experiment was normalized as 100%. This data is also summarized in FIG. 11 for 10 different bone graft samples.

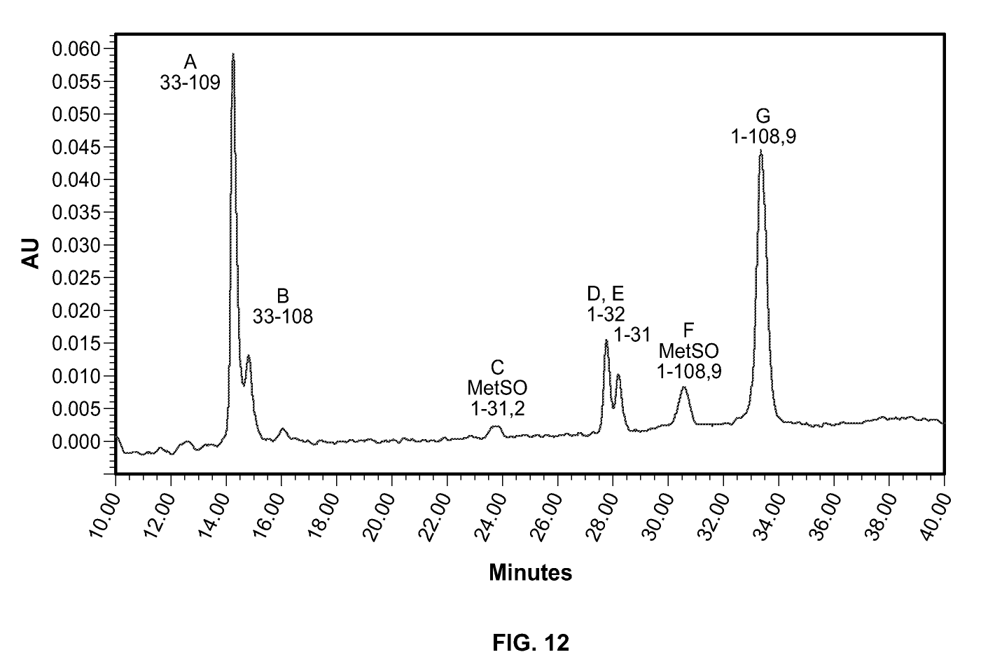

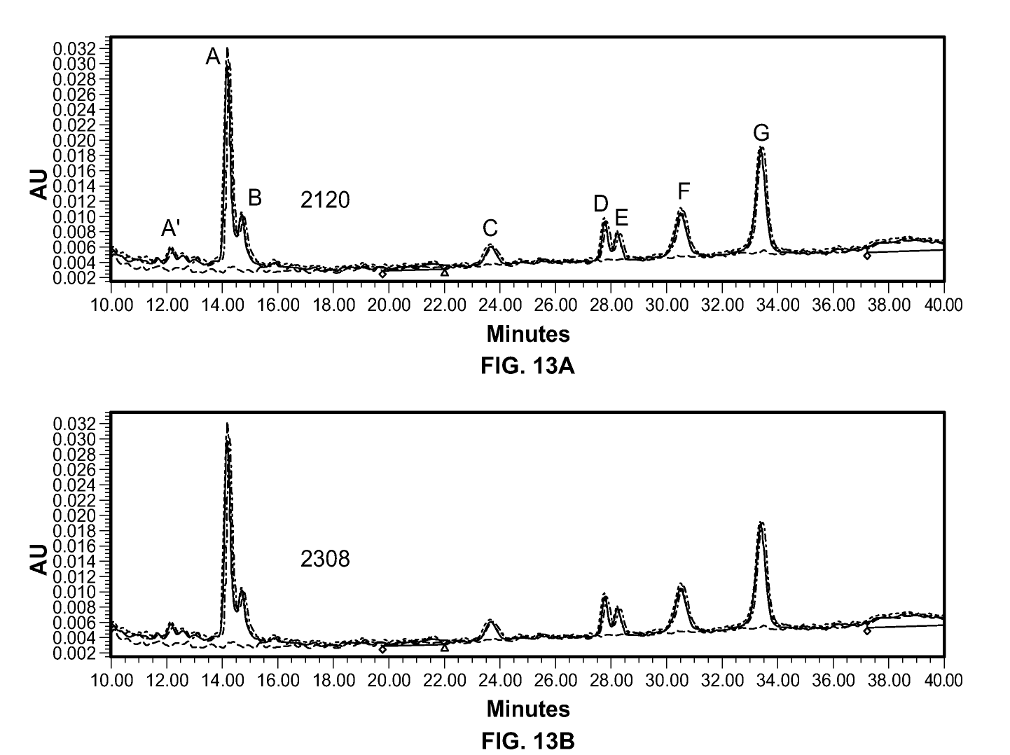

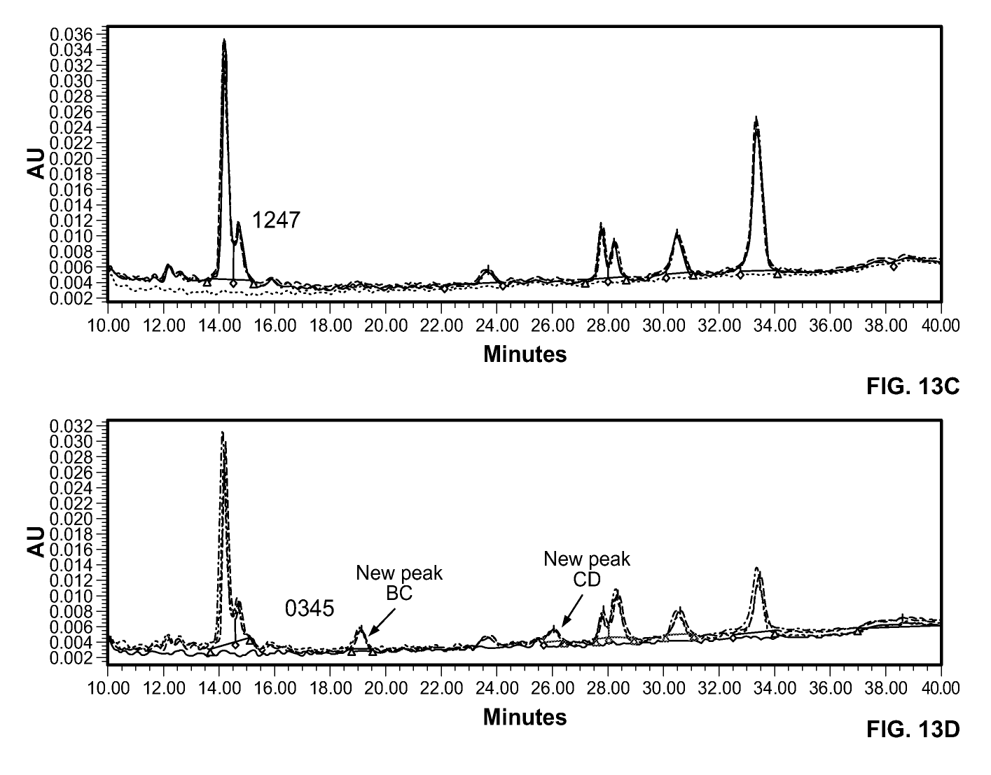

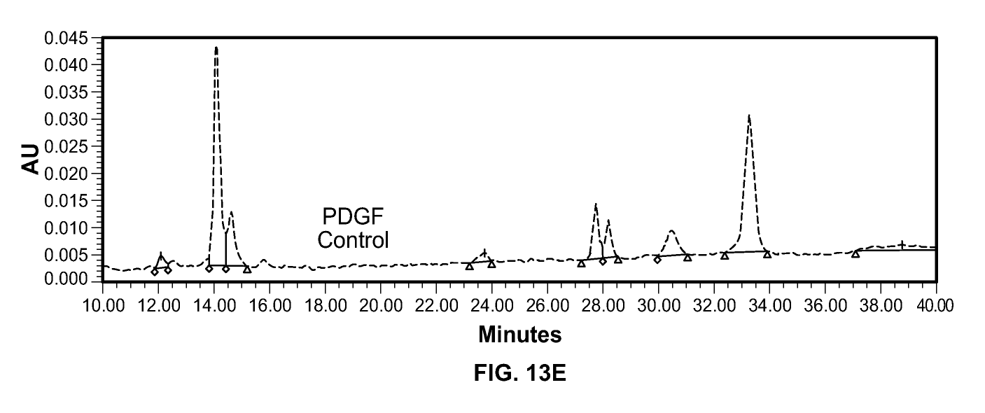

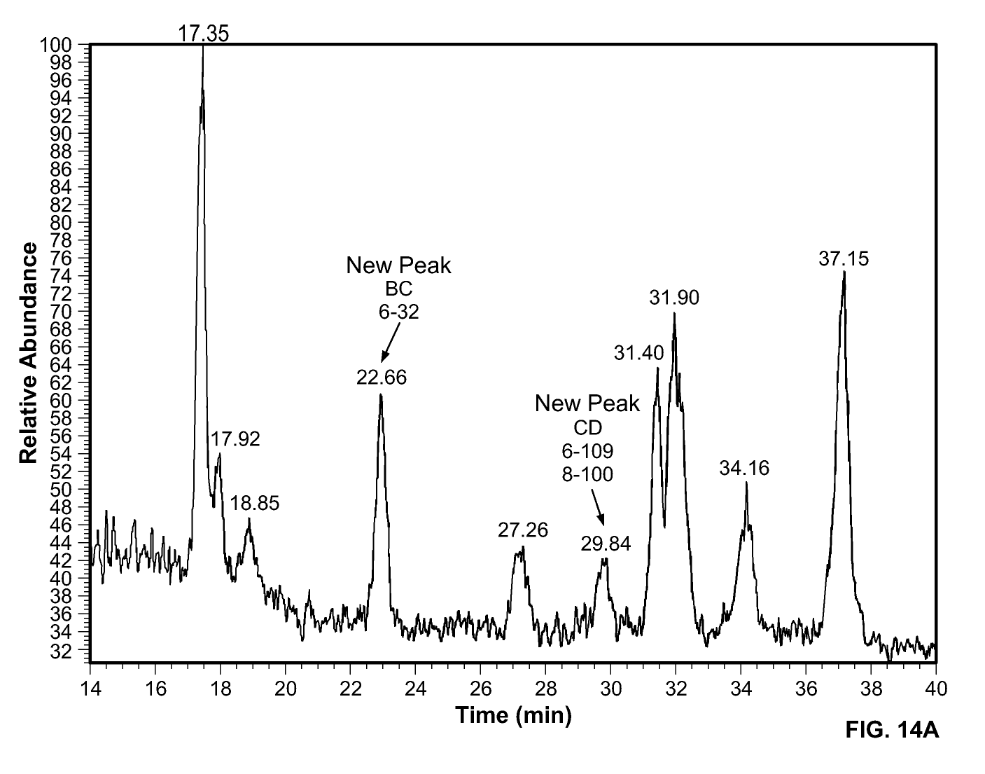

Reversed phase HPLC was also used to quantify the amount of PDGF and changes in its structure due to proteolytic cleavage and/or chemical modification (FIGS. 12 and 13A-13E). Samples were reduced by 200 mM DTT and 4 M guanidine HCl, pH 8.8 for 5 minutes at 50° C. Reversed phase HPLC was performed using a Vydac C₁₈ column 5 μm 4.6 mm×250 mm with a 5 μm guard cartridge (Grace Davison Discovery Sciences, Hesperia, Calif.) using a gradient of 24-80% acetonitrile in 0.06% trifluoracetic acid for 60 minutes at a flow rate of 1.2 ml/min at 37° C. FIG. 12 illustrates several possible PDGF fragments or chemically modified polypeptides. For FIGS. 13A-13D, triplicate runs are compared to a PDGF control (FIG. 13E).

PDGF cleavage products were also identified using ESI LC/MS on Thermo LCA Deca XP MAX, with Tune and Xcalibur software after separation on the Vydac C₁₈ column 5 μm 4.6 mm×250 mm with a 5 μm guard cartridge (Grace Davison Discovery Sciences, Hesperia, Calif.) using Agilent 1100 series, with Agilent G1312A Bin Pump, Agilent G1329A ALS, Agilent G1330B ALS, and Agilent G1314A VWD. Data was acquired from m/z 200-2000 for MS mode (FIGS. 14A and 14B).

### Example 3

### Binding and Release of PDGF from a Bone Graft

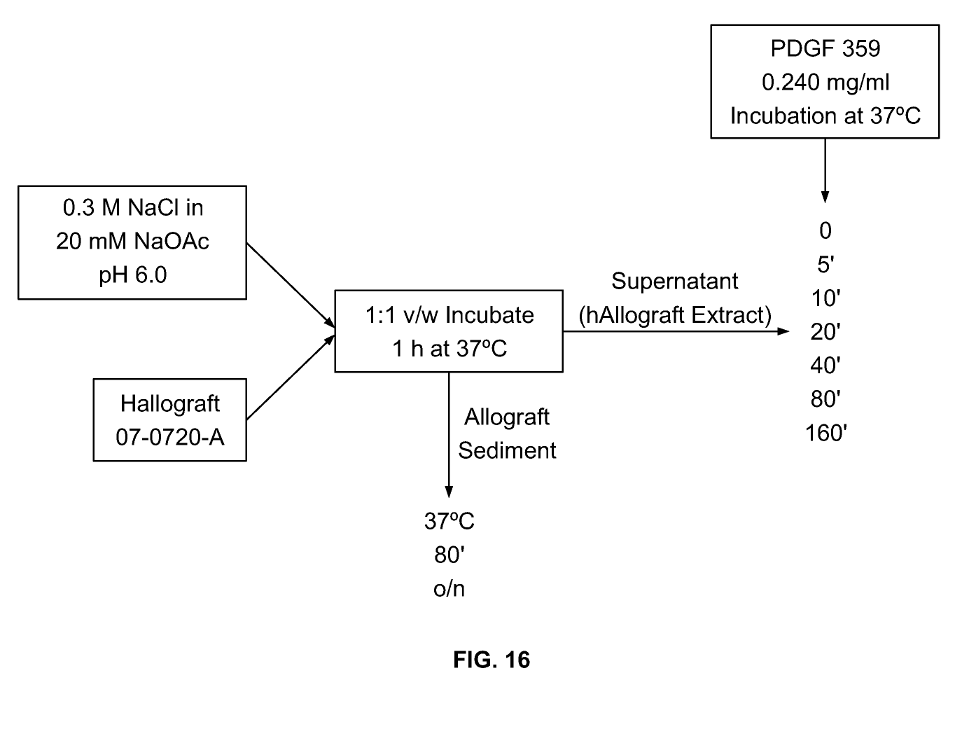

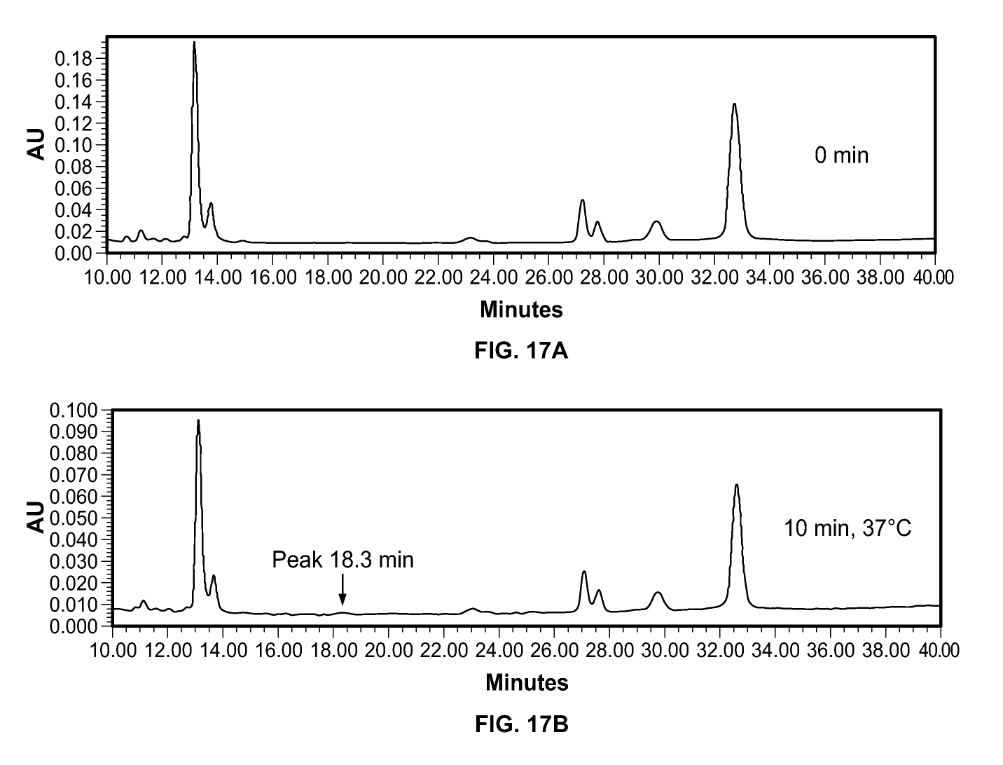

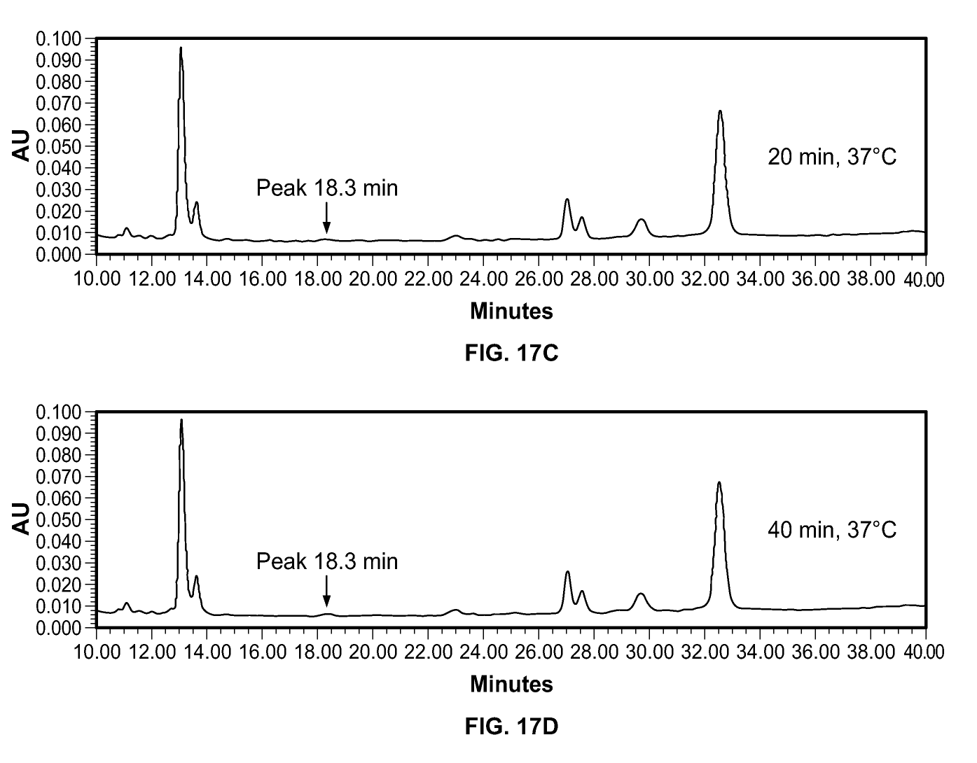

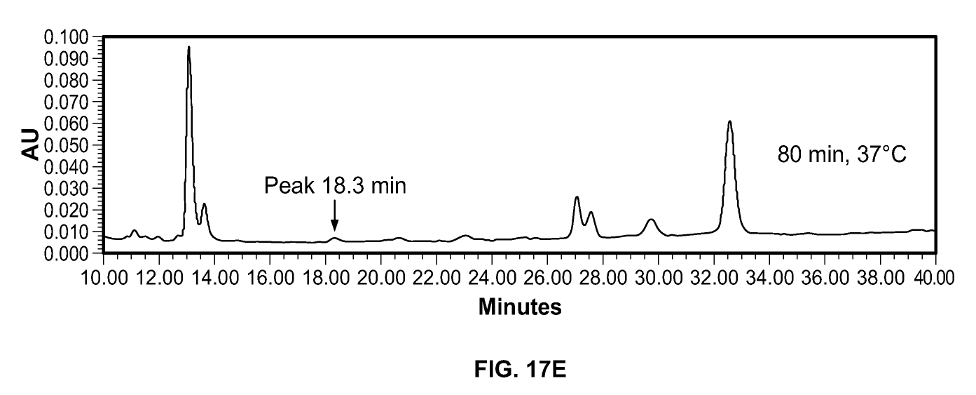

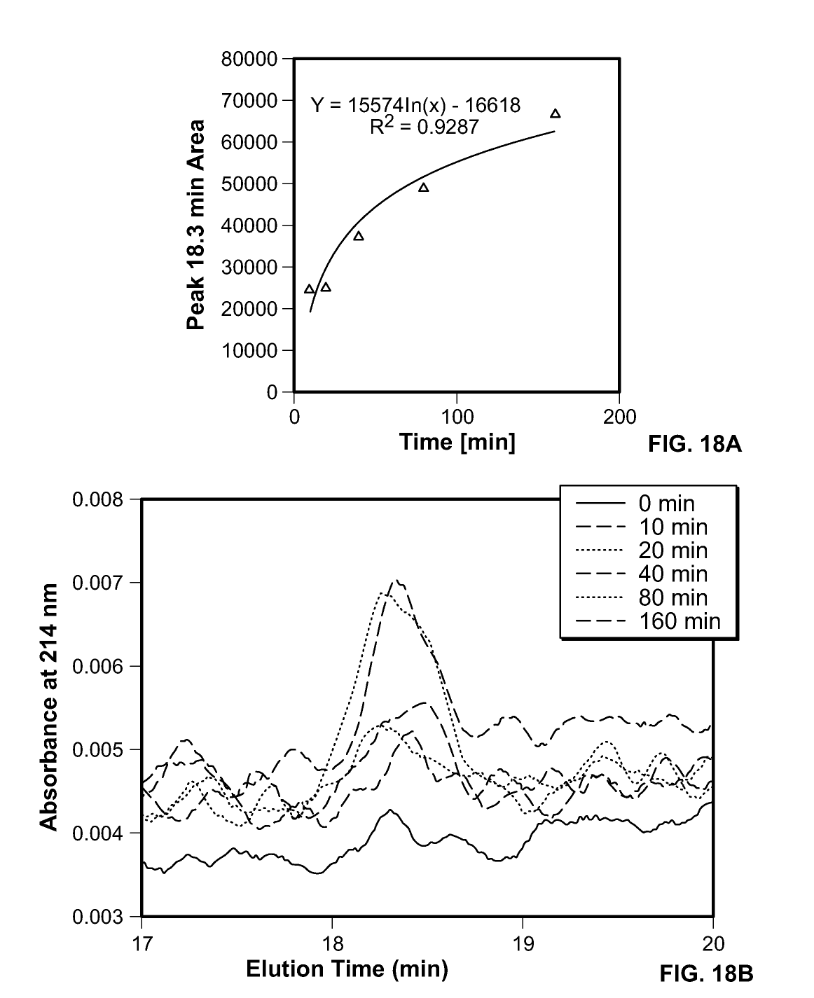

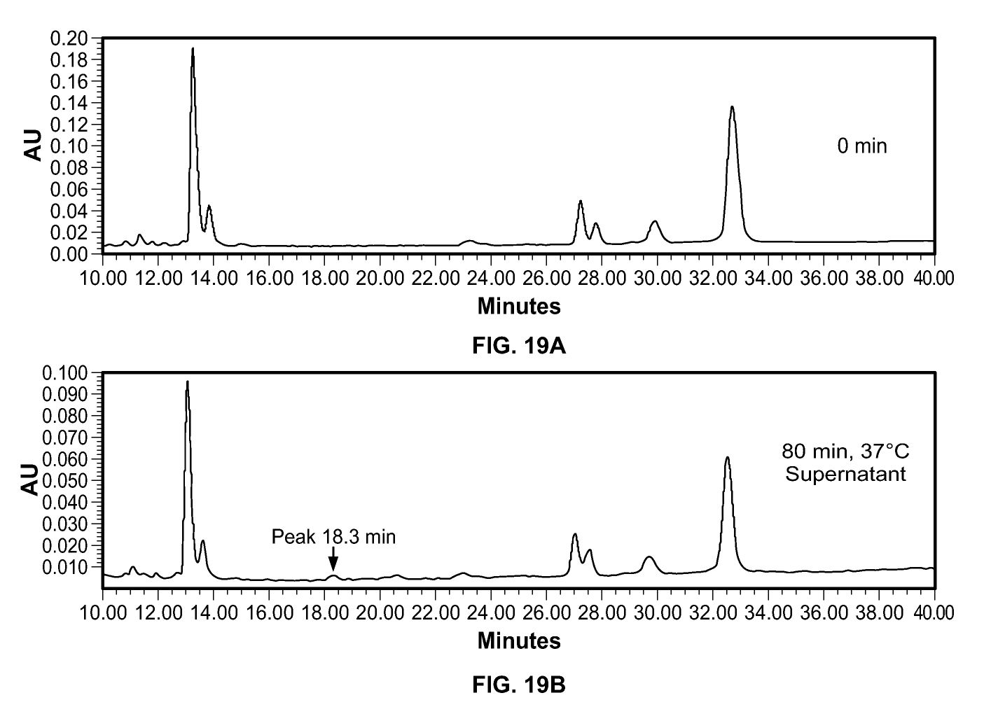

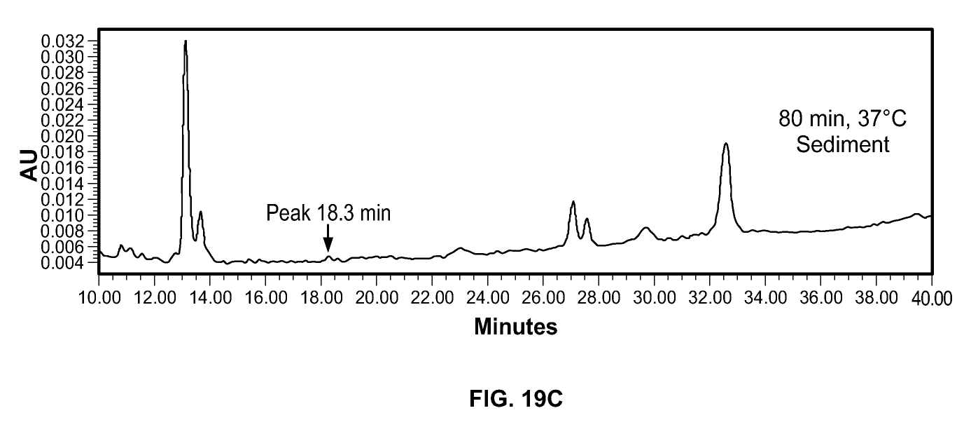

FIG. 16 summarizes an exemplary protocol for studying the removal of protease activity from bone graft. In particular, a 1:1 (volume/weight) mixture of (i) 0.3 M NaCl in 20 mM NaOAc, pH 6.0 and (ii) human bone graft sample 07-0720-A were incubated for one hour at 37° C. Then, the solid bone graft was sedimented using standard methods to separate it from the liquid supernatant (bone graft extract). The solid bone graft sediment was incubated with PDGF at 0.240 mg/mL at 37° C. for either 80 minutes or overnight. The supernatant (bone graft extract) was incubated with PDGF at 0.240 mg/mL at 37° C. for either 0, 5, 10, 20, 40, 80, or 160 minutes. Reversed phase HPLC was then performed on the samples as described in Example 2. Reversed phase HPLC profiles of PDGF incubated for different times with supernatant (bone graft extract) are shown in FIGS. 17A-17E. PDGF cleavage by bone graft extract/supernatant was time dependent (FIGS. 18A and 18B). Reversed phase HPLC profiles of PDGF incubated for different times with bone graft sediment are shown in FIGS. 19A-19C. In FIGS. 17A-17E and 19A-19C, the peak at 18.3 minutes represents cleaved PDGF. FIGS. 19A-19C indicate that little protease activity remains in the bone graft sediment after the 0.3 M salt elution.

### Example 4

### Binding and Release of PDGF from a Bone Graft

An exemplary assay for protease activity is described below using the QuantiCleave™ Protease Assay Kit, Pierce, Cat. #23263. Human bone graft sample 07-0720 was included in a suspension of 80% bone graft and 20% 13-TCP (similar to that described in Example 1). Assay buffer was prepared by dissolving a BupH borate buffer pack in 500 ml of DI water to make 50 mM borate, pH 8.5. Succinylated casein solution was prepared by dissolving one vial (10 mg) of lyophilized succinylated casein in 5 ml of allograft resuspension buffer to make a 0.2 mg/ml solution (this solution can be used for 48 samples in a 96 well microplate). Trypsin stock solution was prepared by dissolving lyophilized TPCK Trypsin in 1 ml of the assay buffer to make a 50 mg/ml stock solution. Aliquots (10-50 uL) of this stock were frozen and stored at −80° C. A trypsin standard was prepared by serial dilution of the trypsin standard starting from 10 ug/ml. TNBSA working solution was prepared by adding 100 uL of supplied TNBSA stock solution to 14.9 ml of the assay buffer. Allograft resuspension buffer (ARB) was prepared by mixing one volume of 20 mM NaOAc, pH 6.0 with two volumes of 20 mM NaOAc and 1 M NaCl. EDTA 100 mM stock solution was prepared by weighing 2.92 g of EDTA and adding 80 ml water. This EDTA solution was titrated with 2.5 M NaOH to pH 7.00, and the final volume was adjusted to 100 ml.

The 80% bone graft and 20% TCP suspension was weighed (50, 25, or 12.5 mg) into 1.5 ml centrifuge tubes, and 200 uL of allograft resuspension buffer was added. Substrate solution (100 uL) was incubated with 50 uL of trypsin standard or 50 uL of bone graft supernatant (prepared as described in Example 2) and 200 uL of the succinylated casein substrate with mixing at room temperature for 20 minutes. TNBS (50 uL) was added to each standard and the supernatant. TNBS (100 uL) was added to each bone graft suspension. Mixtures were incubated for another 20 minutes at room temperature. Bone grafts were centrifuged, and 200 uL of the resulting supernatants were added to the microplate with the trypsin standards. Absorbance at 450 nm was measured on the Spectramax plate reader (Molecular Devices).

For this assay, the following samples and controls were analyzed. “A” denotes a control bone graft without a wash. A complementary volume of ARB was added just before adding the substrate. “Ac” denotes a control that is the same as “A” except that an equal volume of ARB was added instead of the substrate. “AW” denotes a bone graft incubated with 150 uL of ARB for 60 minutes at room temperature and then washed 3 times with 1 ml NaOAc, pH 6.0, bone graft was separated from supernatant (“AWS)” after spinning at 15,344×g for 2 minutes and then assayed as done for bone graft alone (A). All controls (denoted by “c” in the sample name) were prepared the same as the regular samples, except instead of the protease substrate (solution of succinylated casein) only ARB was added into the incubation mixture. These controls account for the peptides eluted from the bone graft with ARB that give false positive signals with trinitrobensene sulfonic acid (TNBS, which specifically reacts with N-terminal amino groups of peptides giving a signal at 450 nm). The sample values from which those controls were subtracted are shown in FIG. 7. “AWEc” denotes a control that is the same as “AW” except that an equal volume of ARB was added instead of the substrate. “AWES” denotes the supernatant from initial incubation of “AWE” for 60 minutes with 100 uL of ARB. “AWEcSc” denotes a control that is the same as “AWES” except that an equal volume of ARB was added instead of the substrate. Samples denoted by “E” in the sample name were obtained like AW and AWS samples except that the protease assay was performed in the presence of 5 mM EDTA (added with the protease substrate into the reaction mixture).

FIG. 7 shows protease activity measured using this method. The signals from controls of no protease substrate were subtracted to generate the data for this figure since the peptides that eluted from the bone graft produced a large signal in the assay. Protease activity was evenly distributed between the soluble supernatant from the salt wash and insoluble bone graft sediment. The soluble protease is expected to exhibit faster cleavage kinetics due to the enhanced substrate diffusion compared to the insoluble protease remaining on or in the bone graft.

### Example 5

### Exemplary Protease Determination Methods

If desired, standard methods can be used to identify one or more proteases associated with a bone graft and/or one or more other polypeptides present. In particular, the following methods allow the identification of most or all of the polypeptides associated with a bone graft. These polypeptides may include one or more proteases that can cleave a polypeptide of interest (such as PDGF).

In principle, the salt eluents from bone graft prepared as described in Example 3 are concentrated at least ˜100× using a 1000Da cut off ultrafiltration device and then used either directly in an MudPIT (Mulditimensional protein identification technology) LC MS/MS experiment (world wide web at fields.scripps.edu/mudpit/ or cshprotocols.cshlp.org/cgi/content/full/2006/28/pdb.prot4555, which are each hereby incorporated by reference in their entireties, particularly with respect to LC MS/MS methods) or separated on a SDS PAGE, cut off, digested in gel with trypsin, and followed either by LC MS/MS or MALDI MS determination of polypeptides in the sample using standard protocols. In a MudPIT experiment, the diluted sample is loaded on an ionic exchange capillary column. Then the fractions from the column are separated on a C18 column and injected into the mass spectrometer. In some embodiments, trypsin digestion of the bone graft eluate is performed and then the resulting mixture is analyzed using LC MS/MS.

To confirm the data, multiple orthogonal methods may be applied and cross-referenced. In some embodiments, final confirmation of the protease identification is an experiment in which both a polypeptide of interest (such as PDGF) as the substrate and a specific peptide substrate for the suspected protease identified by MS are used. Cleavage by a bone graft salt eluate is compared to cleavage by a purified suspected protease purchased from a vendor. In some embodiments, multiple proteases simultaneously contribute to the proteolytic degradation of a polypeptide of interest (such as PDGF).

For example, the following protocol may be used. This exemplary protocol involves SDS-PAGE protein separation, in gel trypsin digestion, and MALDI MS. If desired, the samples can be scaled up or down, depending on the concentration of protease present in the bone graft.
1) Weigh one aliquot of bone graft (such as LifeNet, # 07-720B-320) into a 15 mL conical tube.
2) Add 1:1 v/w of Resuspension Buffer, mix well, and incubate for 1 hour at room temperature on a rotator.
   a. For the preparation of Resuspension Buffer, mix 1 volume of 20 mM NaOAc solution to 2 volumes 20 mM NaOAc and 1 M NaCl solution.
3) Spin bone allograft samples and controls at maximum speed for 2 minutes.
4) Concentrate the supernatant using a 1000 dalton ultrafiltration device into 100 ul.
5) Run gel to test for presence of a protease.
   a. Combine 10 μL of supernatant from bone graft sample to 10 μL of Laemmli Sample Buffer (BioRad #161-0737).
   b. Incubate at 90° C. for 5 minutes.
   c. Spin sample at 14×g for 1 minute.
   d. Load onto 10-well 12% Tris-HCl gel (BioRad #161-1156).
   e. Run at 200 V for ±30 minutes.
   f. Wash 3 times with H₂O for 20 minutes each wash.
   g. Stain overnight with Coomassie stain (BioRad #161-0786).
6) Process gel using standard methods.

In-gel trypsin digestion for subsequent analysis by mass spectrometry can be performed as follows. For this method, use clean reagents of the highest purity, and dedicate them to this procedure. Shaking and/or sonication is not necessary.

### General Reagents

The following general reagents are used.
1) Ammonium bicarbonate. Dissolve 0.79 g in 100 mL milliQ water, filter sterilize (optional), and place into a tightly-capped bottle. Replace after 1 month.
2) Acetonitrile.
3) Dithiothreitol (DTT, 154.2 g/mol). Make fresh 45 mM solution in a 1.5 mL tube by weighing less than 10 mg and adding appropriate amount of milliQ water. DTT is only necessary if sample has not been previously reduced and alkylated during 2D gel electrophoresis.
4) Iodoacetamide (IA, 185 g/mol). Make fresh 100 mM solution in a 1.5 mL tube by weighing less than 10 mg and adding appropriate amount of milliQ water. IA is only necessary if sample has not been previously reduced and alkylated during 2D gel electrophoresis.
5) Trifluoroacetic acid (TFA) (10×1 mL ampules, Pierce cat #28904). In a fume hood, open a 1 mL ampule and mix 50:50 with milliQ water for a 50% stock solution, then dilute 1:5 in milliQ water to make a 10% working stock solution. All can be stored at −20° C.
6) Modified trypsin (5 vials each containing 20 μs lyophilized typsin; Promega cat #V5111, use trypsin gold). Resuspend to make a working 0.1 mg/mL stock solution. Carefully aliquot 10×20 μL into 0.5 mL tubes and store at −20° C.

### Reagents Specific to Silver-Stained Proteins

The following reagents are specific to silver-stained proteins: potassium ferricyanide and sodium thiosulfate. Dissolve 50 mg potassium ferricyanide and 80 mg sodium thiosulfate in 5 mL milliQ water in a clean 15 mL tube. This solution is unstable and should be made fresh every time and used within 30 minutes.

### Reagents Specific to Peptide Clean-Up and Concentration

The following are recommended: 10 μL pipettor (or one that holds 10 μL tips) and ZipTipC18 pipet tips (Millipore cat #ZTC18S096).

### Working Solutions

The following working solutions usually only require 1 mL for 10-20 reactions. Make fresh daily:
1) 50 mM ammonium bicarbonate, 50% acetonitrile. Mix equal volumes of 100 mM ammonium bicarbonate and 100% acetonitrile.
2) 25 mM ammonium bicarbonate. Combine 250 μL 100 mM ammonium bicarbonate and 750 μL milliQ water.
3) 0.1% trifluoroacetic acid (TFA). Dilute 10 μL 10% TFA into 990 μL milliQ water.
4) 60% acetonitrile, 0.1% TFA. Combine 600 μL acetonitrile, 390 μL milliQ water, and 10 μL 10% TFA.

Pipet tips do not need to be changed between samples for steps 1-6 below. There is minimal risk of cross-contamination of samples until trypsin protease has entered the gel slices and begin to digest the samples. Shaking and/or sonication is not necessary.

Step 1. Band Excision. For wet gels, insert the corner edge of a straight razor at the top corner of the band and chop down along the length of the band (do not slice razor through gel). It is best to minimize excess gel and better to waste a bit of protein if necessary. This helps reduce background (and increase overall protein concentration). Chop sides of band, and then cut band into 1 mm cubes and place into a 0.5 mL tube. For gels archived in acetate sheets, slice bands using the corner edge of a straight razor and place into a 0.5 mL tube.

Step 2. Equilibration. Use 100 μL of 50 mM ammonium bicarbonate for 15 minutes. For gels archived in acetate sheets, remove the acetate pieces using forceps, remove reswelled gel slice, cut into 1 mm cubes, and place back into tube. Discard the wash.

A separate step is not needed to de-stain coomassie blue-stained proteins. Sufficient de-staining is accomplished during processing steps 4-5.

Step 3. Silver removal (adapted from Gharandaghi et al., Electrophoresis 20:601). Add 100 μL of the potassium ferricyanide/sodium thiosulfate solution to the tube containing the gel slices and let sit for 5 minutes (or longer if brownish color is not removed from the gel). Discard the solution and replace with excess (250 μL) 100 mM ammonium bicarbonate for 10 minutes. Remove the ammonuim bicarbonate and replace with 100 mM ammonuim bicarbonate for 10 minutes. Repeat this ammonium bicarbonate washing until the yellow coloration is gone from the gel slices. Discard last wash.

Step 4. Reduction/Alkylation. (This step can be skipped if samples come from a 2DE protocol which includes reduction and alkylation). Add 150 μL 50 mM ammonium bicarbonate to sample. Add 10 μL 45 mM DTT and incubate at 50° for 15 minutes. Then add (to the same tube) 10 μL of 100 mM iodoacetamide and place in the dark at room temperature for 15 minutes. This results in carbamidomethylation of the cysteine residues (net addition of 57 daltons to each cysteine residue).

Step 5. Equilibration/Dehydration. Replace liquid with 50-100 μL 100% acetonitrile for 10 minutes, or until the gel slices turn white (may have to repeat once). Remove liquid and desiccate for 5 minutes in a vacuum centrifuge. Dehydrated gel slices can be stored for months in capped 0.5 mL tubes at −20° C.

As an optional step, prior to 100% acetonitrile, remove liquid and replace with 100 μL 50% acetonitrile, 25 mM ammonium bicarbonate (1:1 mix with 50 mM acetonitrile), and let sit for 15 minutes. This can be repeated once to further remove residual coomassie stain.

Step 6. Digest. Reswell the dehydrated gel slice in 10-15 μL of 0.01 μg/μL modified trypsin (Promega) in 12.5 mM ammonium bicarbonate. Dilute the appropriate amount of 0.1 mg/mL stock trypsin solution 1:10 into 12.5 mM ammonium bicarbonate and carefully add 10-15 μL per sample. This is the critical step when trypsin enters the gel; use only what is necessary to cover the gel slices. It is better to have all of the solution enter the gel than to have excess remaining after 20 minutes. Digestion is complete by 2 hours at 37°, or can go overnight if necessary.

Starting with step 7, use a fresh pipet tip for every sample.

Step 7. Overlay (optional). If necessary, after all of the trypsin solution has entered the gel slice (−20 minutes), add additional 12.5 mM ammonium bicarbonate (without additional trypsin) at 5 μL intervals until the gel slice is just covered (do not add excess).

Step 8. Peptide Extraction. Remove the supernatant, which may contain some of the peptides that have diffused out of the gel slices, to a new labeled 0.5 mL tube. Extract peptides from the gel slice with 15-25 μL 60% acetonitrile, 0.1% TFA. After 15 minutes, remove the extract and combine with the supernatant in the new tube, and repeat with a second extraction as above. When transferring the second extraction into the new tube containing the first extraction and the supernatant, pipet up and down a few times to ensure complete mixing of the reagents. Finally, dry down the extract/supernatant in the new tube using a vacuum centrifuge to dryness, but don't let it go too long after the liquid has evaporated (times vary with different vacuum centrifuges, and could take anywhere from 30-60 minutes using the volumes listed above; check as necessary).

Step 9. Reconstitution and Mass Analysis. Dissolve peptides in 4 μL 0.1% TFA. Samples can be directly analyzed by LC/MS. Abundant samples can be analyzed directly by MALDI-TOF MS, less abundant samples usually require clean-up and concentration using ZipTipC18 (Millipore, catalog number ZTC18S096) pipette tips (requires a 10 μL pipettor):
a. Equilibrate tip in 60% acetonitrile, 0.1% TFA (2×10 μL).
b. Wash in 0.1% TFA (2×10 μL).
c. Load up to 10 μL sample with repetitive pipetting (−5 times).
d. Wash in 0.1% TFA (2×10 μL).
e. Elute into 2 μl 60% acetonitrile, 0.1% TFA with repetitive pipetting. (2 μL placed into fresh tube prior to elution).

Step 10. Sample preparation for MALDI-TOF MS. Apply 0.4 μL peptide mixture to a MALDI target, and overlay with 0.4 μL α-cyano-4-hydroxycinnamic acid matrix (5 mg/mL in 60% acetonitrile and 0.1% TFA, supplemented with 1 mg/mL ammonium citrate).

MALDI-TOF MS. Peptide mixtures were analyzed by matrix-assisted laser desorption time of flight (MALDI-TOF) and TOF/TOF tandem mass spectrometry using an Voyager 4700 mass spectrometer (Applied Biosystems, Framingham Mass.). Mass spectral data, in the form of peptide mass maps of the intact molecular peptide ions (M+H), as well as fragmentation data derived from individual peptide ions, were used to interrogate the Swiss-Prot and NCBInr protein databases for statistically significant protein matches using GPS Explorer software (Applied Biosystems) running the MASCOT search engine (Matrix Science). Trypsin autolytic peptide ions m/z=842.51, 2211.10) were used for internal calibration of the peptide mass maps, allowing for searches to be performed with mass accuracy of less than 20 ppm. Searches also allowed for 1 missed cleavage, complete carbamidomethylation of cysteine sulfhydryls, and partial oxidation of methionine residues.

### Example 6

### Analysis of Protein Components in Mineralized Human Bone Allograft by Mass Spectrometry

The objective of this study was to identify major components of the peptide fraction eluted from human allograft under the conditions of elution of rhPDGF-BB from this material, and compare by LC/MS/MS the peptide profiles of the eluates from the allografts with and without proteolytic activity, identifying potential candidates for proteases causing the cleavage of rhPDGF-BB.

### Materials and Equipment

The following materials were used to conduct the testing:

Test Materials & Lot Number

Ground Cortical Bone, Particle size = 250-1000 μm, Manufacturer =
LifeNet BN: 07-0720
Ground Cortical Bone, Particle size = 250-1000 μm, Manufacturer =
LifeNet BN: 07-2518
Ground Cortical Bone, Particle size = 250-1000 μm, Manufacturer =
LifeNet BN: 06-5726
Ground Cortical Bone, Particle size = 250-1000 μm, Manufacturer =
LifeNet BN: 06-1247

The following supplies were used to conduct the testing:

| Supplies | Catalogue Number |
|---|---|
| 1000 MWCO centrifuge filters | Pall, catalogue # OD001C41 |
| BCA Protein Assay Reagent A | Pierce, catalogue # 23228 |
| BCA Protein Assay Reagent B | Pierce, catalogue # 1859078 |
| Albumin Standard, 2.0 mg/mL | Pierce, catalogue # 23209 |
| Sodium Chloride (NaCl), reagent | Fisher, catalogue # S271-10 |
| grade or equivalent | |
| Trichloroacetic Acid (TCA), ACS | Sigma-Aldrich, catalogue # |
| grade or equivalent | T6399-5G |
| Acetone | Burdick and Jackson, catalogue # |
| | 67-64-1 |
| Eppendorf International 1.5 mL | VWR, catalogue # 89000-040 |
| Standard Micro Test Tube, or | |
| equivalent | |
| Tris(hydroxymethyl)aminomethane | Fisher, catalogue # BP152-500 |
| [Tris (Base)] | |
| Urea | Fisher, catalogue # BP169-500 |
| Water, HPLC grade or equivalent | J. T. Baker, catalogue # |
| | 4218-02 |
| Bond Breaker ™ TCEP Solution | Fisher, catalogue # 77720 |
| Iodoacetamide (IAA) | Sigma-Aldrich, catalogue # |
| | I1149-25G |
| Calcium Chloride (CaCl₂) | Sigma-Aldrich, catalogue # |
| | C5670-100G |
| Hydrochloric Acid (HCl), ACS | EMD, catalogue # HX0607-1 |
| grade or equivalent | |
| Formic Acid | EMD, catalogue # 11670-1 |
| Fused silica, 360 OD, 100 ID | Polymicro Technologies, |
| | catalogue # TSP100375 |
| Methanol, HPLC grade or | VWR, catalogue # JT9093-03 |
| equivalent | |
| Kasil 1, potassium silicate | PQ Corporation |
| Formamide | Sigma-Aldrich, catalogue # |
| | F-5786 |
| Trypsin Gold | Promega, catalogue # V5280 |
| Glacial Acetic Acid, sequencing | Fisher, catalogue # BP1185-500 |
| grade or equivalent | |
| Formic Acid, 0.1% in water, HPLC | VWR, catalogue # JT9834-03 |
| grade or equivalent | |
| Formic Acid, 0.1% in acetonitrile, | VWR, catalogue # JT9832-02 |
| HPLC grade or equivalent | |

The following equipment was used to conduct the testing:

| | Equipment |
|---|---|
| Equipment | Identification |
| Beckman Coulter bench top centrifuge | SN: ALP05E15 |
| (Allegra X-15R), or equivalent | |
| Beckman Coulter Microfuge 22R Centrifuge, | Model # 368826 |
| or equivalent | |
| Jupiter HPLC Column, 3 μm C18 300Å | Phenomenex, bulk |
| New Objective High Pressure Cell | New Objective, |
| | catalogue # PIP-500 |
| Jupiter HPLC Column, 5 μm C18 300Å | Phenomenex, bulk |
| Luna HPLC Column, 5 μm SCX 100Å | Phenomenex, bulk |
| Beckman 300 Series pH meter, or equivalent | Beckman Coulter, |
| | catalogue # 511211 |
| Thermo Savant SPD Speedvac SPDIIIV vacuum | Model # |
| centrifuge | SPD111V-115 |
| Thermo Finnigan Surveyor MS Pump Plus HPLC | SN: 50221 |
| Eksigent NanoLC-1Dplus Orbi pump | SN: 07-01-08-163 |
| Thermo Finnigan LTQ | SN: LTQ10129 |
| Thermo Orbi LTQ XL | SN: ORB131274 |

### Sample Preparation

Upon determining the identity of the unknown peaks, human bone allograft samples were chosen to be submitted to a more thorough MuDPIT analysis. Each allograft sample was mixed with 1:3 (w/v) 20 mM NaOAc, pH 6.0 (containing no rhPDGF-BB), and allowed to incubate for 1 hr at RT. Following the incubation, the samples were centrifuged at 14,000 rpm.

The supernatant was removed and split between two 1.5 μL tubes and centrifuged at 14000 rpm for 1 min. The supernatant was then removed from any allograft particulates and added to the 1000 MWCO filter and centrifuged at 4750 rpm overnight or until sample was concentrated to ˜100 μL. Following concentration, the samples were frozen at −80° C.

The concentration of each sample was determined by a BCA protein assay, using albumin as the standard. A calibration curve was made using albumin from 1-1200 μg/mL. Samples were diluted by varying degrees using sample buffer (07-0720 diluted 1:2 & 1:4, 07-2518 diluted 1:10, 06-5726 diluted 1:10, and 06-1247 diluted 1:4). The BCA analysis of each sample was performed, and the volumes of each sample determined to equal 50 μg.

The samples were then used in a TCA precipitation. ⅓ of the current volume determined for 50 μg for each sample was then diluted up to 200 μL with 25% TCA. The samples were incubated at 4° C. for 1 hr. Following incubation the samples were centrifuged at 4° C. for 30 min at 14000 rpm. The supernatant was discarded. The samples were washed with 500 μL cold acetone, and spun again at 14000 rpm at 4° C. for 30 min. Once again the supernatant was discarded and the wash repeated. The protein pellet did not stick to the side of the tube easily, so the supernatant was removed as much as possible and the sample allowed to dry in a vacuum centrifuge.

Following completion of the drying step, the samples were resuspended in 40 μL of 8 M urea and 100 mM Tris-HCl, pH 8.5. To this solution was added 0.4 μL of 500 mM TCEP and incubated for 20 min at RT. Following incubation, the protein samples were alkylated with 0.8 μL of 500 mM iodoacetamide and incubated for 20 min in the dark. The samples were then diluted with 120 μL of 100 mM Tris-HCl, pH 8.5. To the samples were added 1.6 μL 100 mM CaCl₂ and 0.5 μg of trypsin, and incubated for digestion overnight at 37° C. The following morning the samples were removed immediately from the incubator and placed at −80° C.

### RPHPLC Procedure

The HPLC system was set up according to the following conditions: Injection Amount: 12.5 μg; Run Time: ˜120 min/pulse; Gradient: Time 0: % A (100), % B (O), 500 μL/min; Time 10: % A (100), % B (O), 500 μL/min; Time 10.5: % A (100), % B (O), 300 μL/min; Time 115: % A (60), % B (40), 300 μL/min; Time 115.1: % A (100), % B (O), 300 μL/min; Time 120: % A (100), % B (O), 300 μL/min.

### MS Procedure

The instrument was tuned using the instrument and software autotune feature, on the 433 peak of angiotensin. The instrument parameters were determined by the automatic tuning feature on the instrument, and are included in the Appendix.

Tips were pulled from 100 μm ID fused silica. The front reverse-phase column was packed with 5 μm C18 Jupiter resin, and packed tightly using a pressure cell. The tip was packed down and clipped back to 20 cm (the same analytical column was used for all 4 samples).

For preparation of the MuDPIT column, a long piece of fused silica was rinsed under pressure with methanol and dried. The fused silica was then cut into 20 cm pieces. 170 μL of Kasil was combined with 30 μL of formamide and vortexed for 30 s. The fused silica was placed in the Kasil mixture (capillary action creates the frit). The Kasil-infused silica was then baked at 100° C. for 4 hrs. Following the baking procedure the frit was trimmed back to 0.5 cm. The column was then packed with 3-4 cm of SCX Luna resin with a pressure cell; the resin was packed down with methanol under pressure. Another 3-4 cm was then packed with C18 Jupiter resin using the pressure cell. The column was then equilibrated with 0.1% formic acid for 5 min.

Prior to loading the sample onto the column, 40 μL of the sample was removed from the freezer and quenched with 1.5 μL of formic acid. 12.5 μg was then added to the MuDPIT column via pressure cell. The MuDPIT column was then placed inline upstream of the analytical column.

MuDPIT analysis consisted of a series of salt pulses of which 5 μL of 0 mM, 25 mM, 50 mM, 75 mM, 100 mM, 150 mM, 200 mM, 300 mM, 500 mM, 750 mM, and 1000 mM was injected onto the sample. For each salt pulse, the gradient was used as shown in the HPLC gradient above.

### Results

The results of the MuDPIT analysis were complex, with high levels of endogenous proteins such as collagen or keratin. Allograft Lots had the following number of “hits” of each protein in the specific samples, where a protein was identified from a database based on a confirmed peptide sequence.: /0720 (2218); /1247 (755); /2518 (2197); /5726 (929). Allografts 07-0720 and 07-2518 contained considerably more proteins/peptides than allografts 06-5726 and 06-1247. Allografts 07-0720 and 07-2518 are also the two lots that showed high levels of proteolytic cleavage of rhPDGF-BB when the two are combined.

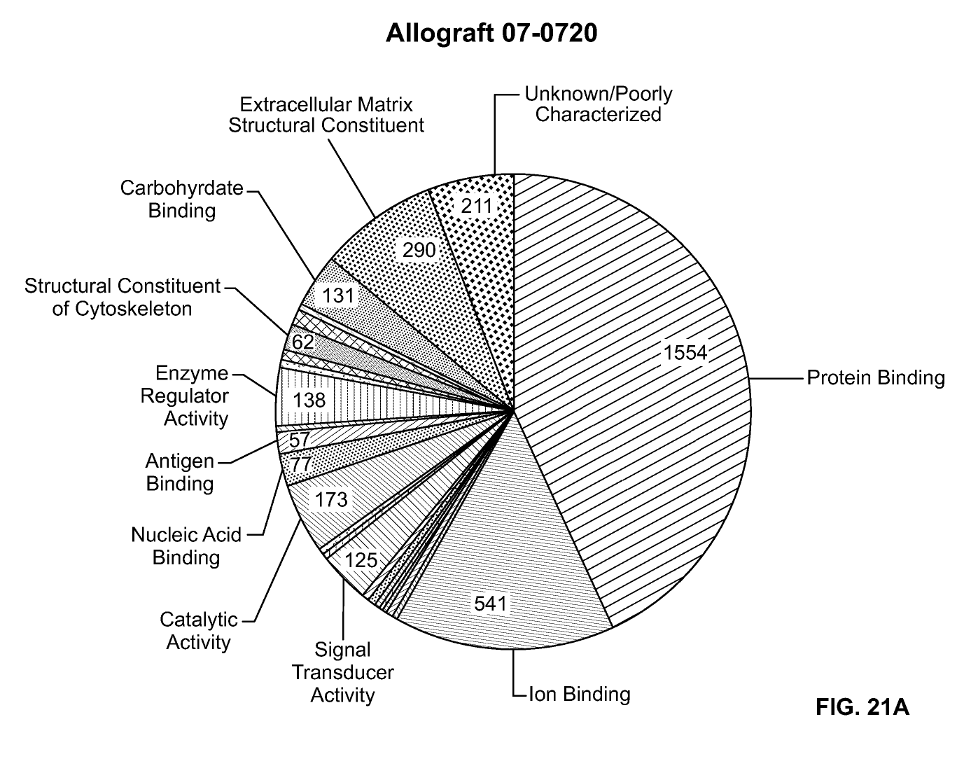

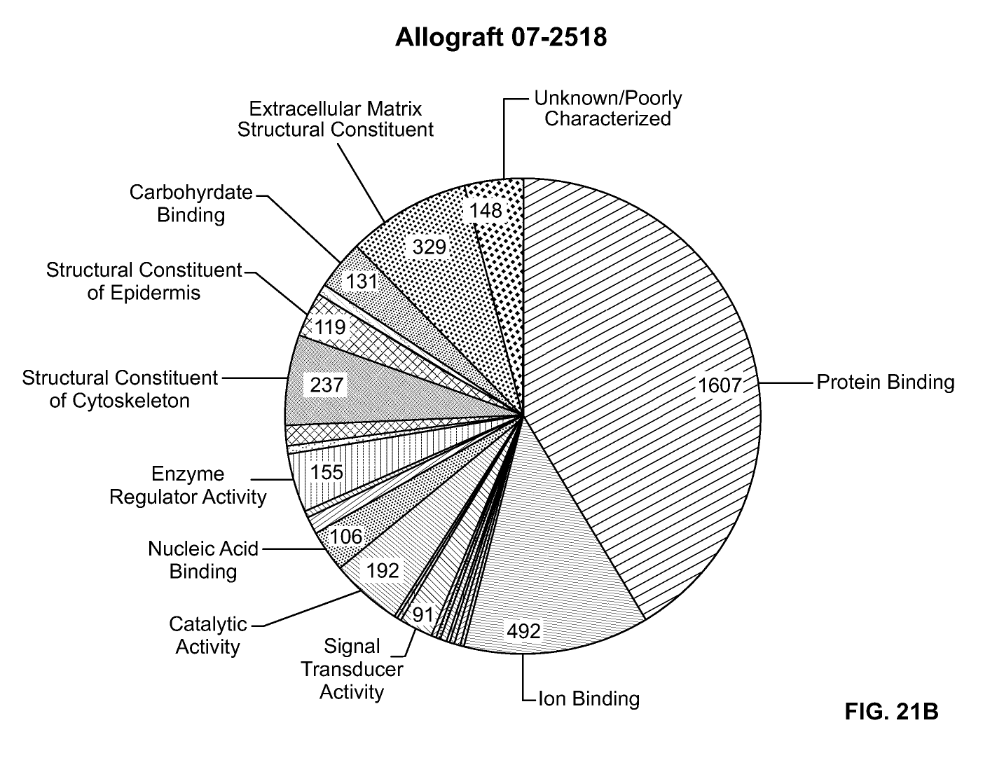

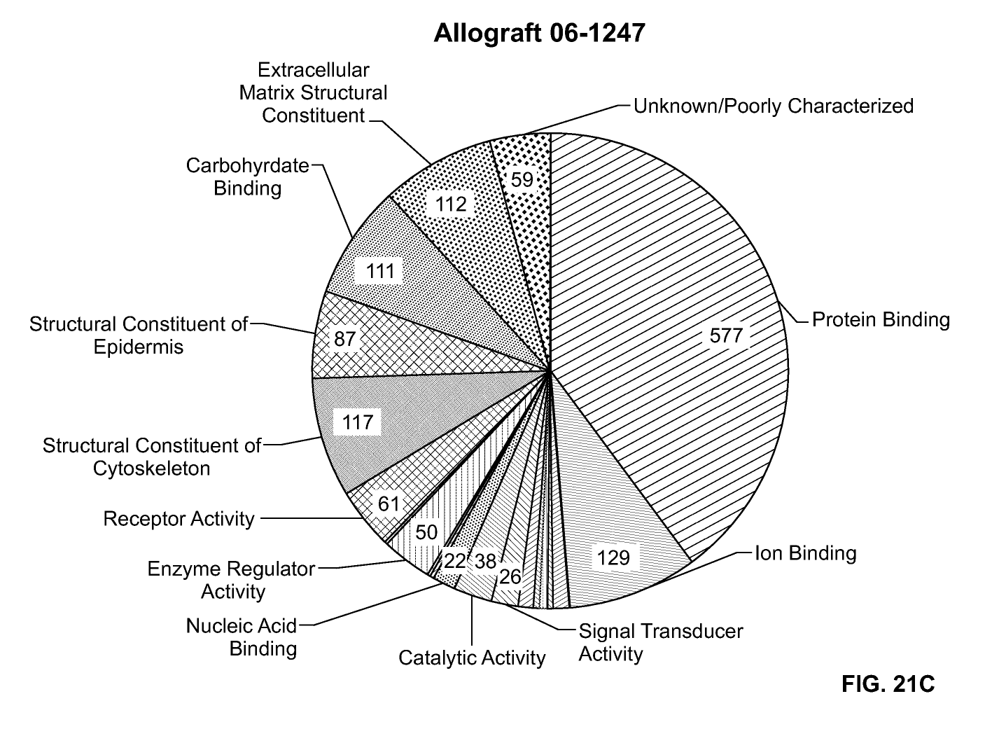

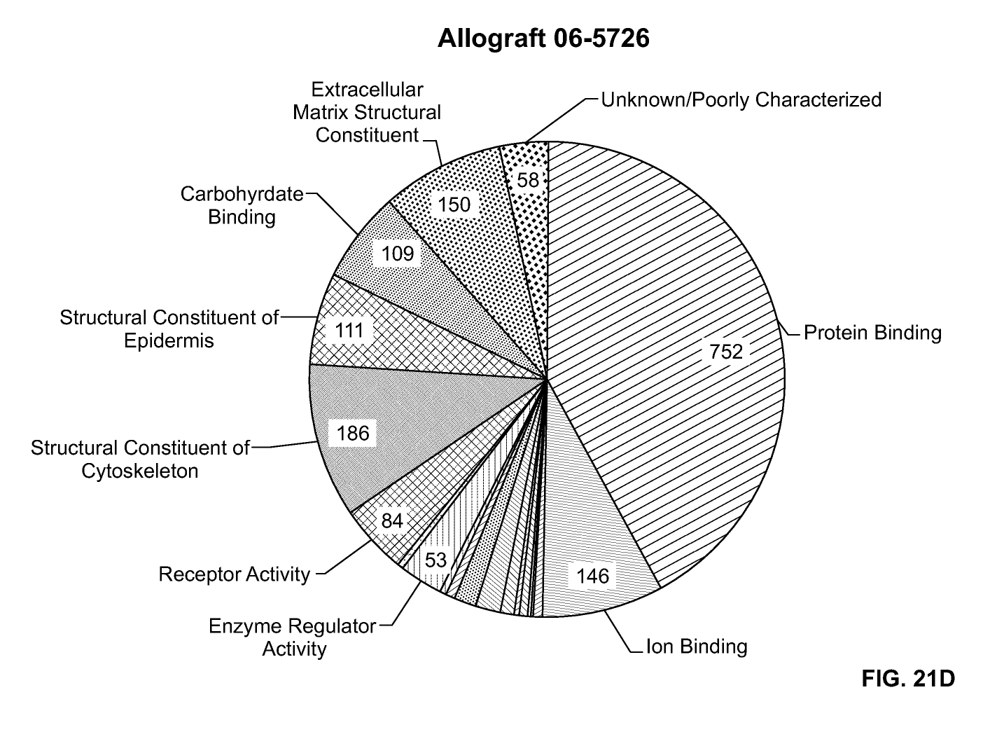

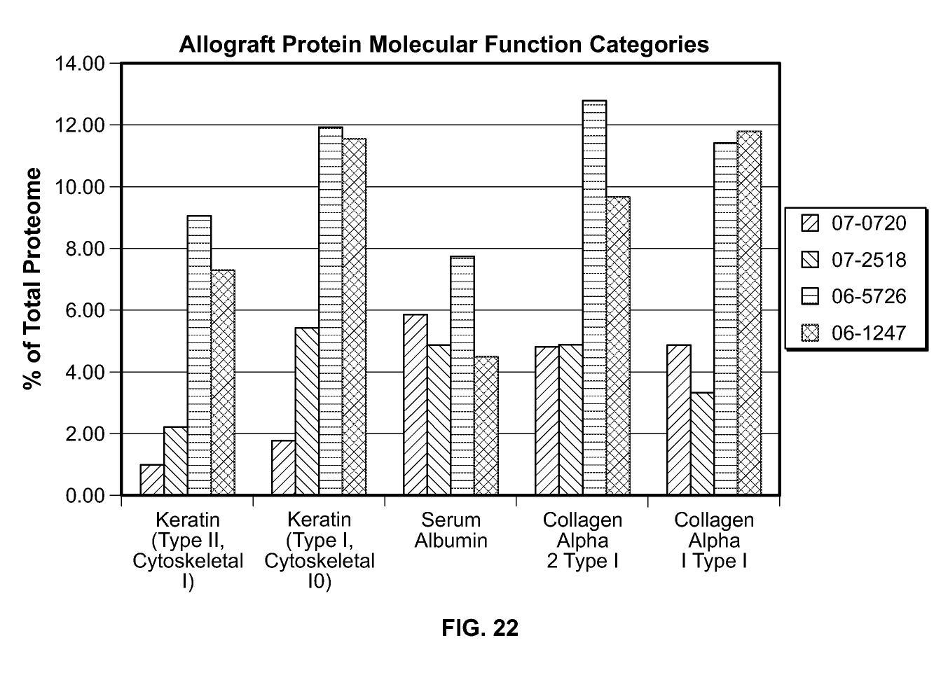

A comparison was made based on Gene Ontology (GO) annotations (http://amigo.geneontology.org/cgi-bin/amigo/browse.cgi, GO database release 2009-07-02) which draws comparisons of proteins based on molecular function, biological process, or cellular component. The allografts which showed high levels of proteolytic cleavage activity (07-0720 and 07-2518) had a higher percentage of proteins that partake in nucleic acid binding, catalytic activity, signal transducer activity, and ion binding (see FIG. 21). In contrast, the allografts that do not show proteolytic cleavage (FIGS. 21C and 21D) had a higher percentage of proteins that are part of tissue structural components. This suggests that some allografts are not cleaned as sufficiently as others, leaving proteins/peptides behind that reactive negatively towards any potential active ingredient. Proteins that make up higher than 5% of at least one allograft sample (keratin, collagen, and serum albumin) were compared (FIG. 22). Samples that have fewer proteins/peptides (and have no proteolytic activity) have higher percentages of these proteins, whereas the more “contaminated” allograft lots have a lower concentration of these proteins.

Three proteases (Cathepsin G, Matrix metalloproteinase-9, Chymase), or proteins known to cause cleavage, were found in samples 07-0720 and 07-2518, and were not detected in samples 06-5726 and 06-1247.

Allografts 07-0720 and 07-2518 also had a higher percentage of other proteins/peptides eluted from the allografts in HPSEC compared to those eluted from the allografts 06-5726 and 06-1247: 87.72 and 90.10 percent, respectively. Conversely, allografts 06-5726 and 06-1247, had 86.14 and 84.03 percent other proteins/peptides, respectively, by HPSEC.

The RPHPLC profile of allograft 07-0720 is not identical to 07-2518, suggesting proteolytic cleavage may have occurred by different proteases between the two samples.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

All references, publications, patents, and patent applications disclosed herein are hereby incorporated by reference in their entirety.

As used herein, the singular form “a”, “an”, and “the” includes plural references unless indicated otherwise.

Reference to “about” a value or parameter herein includes (and describes) embodiments that are directed to that value or parameter per se. For example, description referring to “about X” includes description of “X.”

It is understood that aspect and variations of the invention described herein include “consisting” and/or “consisting essentially of” aspects and variations.

What is claimed is:

1. A method of selecting a bone graft for administration to an individual in conjunction with a polypeptide of interest comprising measuring the protease activity associated with a bone graft, whereby the amount of protease activity associated with the bone graft determines whether the bone graft is selected for administration to the individual in conjunction with the polypeptide of interest.

2. The method of claim 1, further comprising administering the selected bone graft and the polypeptide of interest to the individual.

3. The method of claim 1, wherein the protease activity of two or more bone grafts is measured, and the bone graft with the lowest protease activity is administered to the individual in conjunction with the polypeptide of interest.

4. The method of claim 1, wherein the protease activity of the selected bone graft is less than about 50 trypsin equivalents.

5. The method of claim 1, wherein measuring the protease activity associated with a bone graft comprises:
(a) removing at least a portion of the total amount of a protease associated with the bone graft from the bone graft; and
(b) measuring the amount of a polypeptide substrate that is cleaved by the removed protease, thereby determining the amount of protease activity associated with the bone graft.

6. The method of claim 5, wherein step (a) comprises increasing the ionic strength of the solution comprising the bone graft and protease.

7. The method of claim 6, wherein step (a) comprises incubating the bone graft and protease in a salt solution.

8. The method of claim 7, wherein the salt solution is a NaCl solution.

9. The method of claim 8, wherein the salt solution contains between about 0.15 M NaCl and about 1.5 M NaCl.

10. The method of claim 9, wherein the salt solution contains about 0.3 M NaCl.

11. The method of claim 5, wherein step (b) comprises separating the cleaved polypeptide substrate, the uncleaved polypeptide substrate, and the bone graft.

12. The method of claim 11, wherein high-performance liquid chromatography is used to separate the cleaved polypeptide substrate, the uncleaved polypeptide substrate, and the bone graft.

13. The method of claim 11, wherein size exclusion chromatography is used to separate the cleaved polypeptide substrate, the uncleaved polypeptide substrate, and the bone graft.

14. The method of claim 1, wherein measuring the protease activity associated with a bone graft comprises measuring the amount of a polypeptide substrate that is cleaved by a protease activity associated with the bone graft.

15. The method of claim 14, wherein measuring the amount of cleaved polypeptide substrate comprises:
(a) incubating the polypeptide substrate with the bone graft,
(b) removing at least a portion of the total amount of cleaved polypeptide substrate from the bone graft, and
(c) measuring the amount of cleaved polypeptide substrate.

16. The method of claim 15, wherein step (b) comprises increasing the ionic strength of the solution comprising the bone graft and the polypeptide substrate.

17. The method of claim 16, wherein step (b) comprises incubating the bone graft and the polypeptide substrate in a salt solution.

18. The method of claim 16, wherein the salt solution is a NaCl solution.

19. The method of claim 18, wherein the salt solution contains between about 0.15 M and about 2.0 M NaCl.

20. The method of claim 19, wherein the salt solution contains about 0.6 M NaCl.

21. The method of claim 15, wherein step (c) comprises separating the cleaved polypeptide substrate, the uncleaved polypeptide substrate, and the bone graft.

22. The method of claim 21, wherein high-performance liquid chromatography is used to separate the cleaved polypeptide substrate, the uncleaved polypeptide substrate, and the bone graft.

23. The method of claim 21, wherein size exclusion chromatography is used to separate the cleaved polypeptide substrate, the uncleaved polypeptide substrate, and the bone graft.

24. The method of claim 5 or 14, wherein the polypeptide of interest and the polypeptide substrate are the same.

25. The method of claim 5 or 14, wherein the polypeptide of interest and the polypeptide substrate are different.

26. The method of claim 1, wherein the polypeptide of interest is platelet derived growth factor (PDGF).

27. A method of selecting a bone graft for administration to an individual in conjunction with PDGF comprising selecting a bone graft with a protease activity of less than about 50 trypsin equivalents for administration to an individual in conjunction with PDGF.

28. A method for measuring the protease activity associated with a bone graft, the method comprising:
(a) removing at least a portion of the total amount of a protease associated with the bone graft from the bone graft; and
(b) measuring the amount of a polypeptide substrate that is cleaved by the removed protease, thereby determining the amount of protease activity associated with the bone graft.

29. The method of claim 28, wherein step (a) comprises increasing the ionic strength of the solution comprising the bone graft and protease.

30. The method of claim 29, wherein step (a) comprises incubating the bone graft and protease in a salt solution.

31. The method of claim 30, wherein the salt solution is a NaCl solution.

32. The method of claim 31, wherein the salt solution contains between about 0.15 M NaCl and about 1.5 M NaCl.

33. The method of claim 32, wherein the salt solution contains about 0.3 M NaCl.

34. The method of claim 28, wherein step (b) comprises separating the cleaved polypeptide substrate, the uncleaved polypeptide substrate, and the bone graft.

35. The method of claim 34, wherein high-performance liquid chromatography is used to separate the cleaved polypeptide substrate, the uncleaved polypeptide substrate, and the bone graft.

36. The method of claim 34, wherein size exclusion chromatography is used to separate the cleaved polypeptide substrate, the uncleaved polypeptide substrate, and the bone graft.

37. The method of claim 34, wherein the polypeptide substrate is PDGF.

38. A method for measuring the protease activity associated with a bone graft, the method comprising:
(a) removing at least a portion of the total amount of a protease associated with the bone graft from the bone graft by incubating the bone graft in a salt solution containing about 0.15 M NaCl to about 1.5 M NaCl; and
(b) measuring the amount of PDGF that is cleaved by the removed protease, thereby determining the amount of protease activity associated with the bone graft.

39. A method for measuring the protease activity associated with a bone graft, the method comprising measuring the amount of a polypeptide substrate that is cleaved by a protease activity associated with the bone graft.

40. The method of claim 39, wherein measuring the amount of cleaved polypeptide substrate comprises:
(i) incubating the polypeptide substrate with the bone graft,
(ii) removing at least a portion of the total amount of cleaved polypeptide substrate from the bone graft, and
(iii) measuring the amount of cleaved polypeptide substrate.

41. The method of claim 40, wherein step (ii) comprises increasing the ionic strength of the solution comprising the bone graft and the polypeptide substrate.

42. The method of claim 41, wherein step (ii) comprises incubating the bone graft and the polypeptide substrate in a salt solution.

43. The method of claim 42, wherein the salt solution is a NaCl solution.

44. The method of claim 43, wherein the salt solution contains between about 0.15 M and about 2.0 M NaCl.

45. The method of claim 44, wherein the salt solution contains about 0.6 M NaCl.

46. The method of claim 40, wherein step (iii) comprises separating the cleaved polypeptide substrate, the uncleaved polypeptide substrate, and the bone graft.

47. The method of claim 46, wherein high-performance liquid chromatography is used to separate the cleaved polypeptide substrate, the uncleaved polypeptide substrate, and the bone graft.

48. The method of claim 46, wherein size exclusion chromatography is used to separate the cleaved polypeptide substrate, the uncleaved polypeptide substrate, and the bone graft.

49. The method of claim 46, wherein the polypeptide substrate is PDGF.

50. A method for measuring the protease activity associated with a bone graft, the method comprising
(i) incubating PDGF with the bone graft,
(ii) removing at least a portion of the total amount of cleaved PDGF from the bone graft, and
(iii) measuring the amount of cleaved PDGF.

51. A method for decreasing the protease activity associated with a bone graft comprising removing at least a portion of the total amount of a protease associated with the bone graft from the bone graft.

52. The method of claim 51, further comprising measuring the amount of protease that remains associated with the bone graft.

53. The method of claim 51, wherein removing the protease comprises increasing the ionic strength of the solution comprising the bone graft and protease.

54. The method of claim 53, wherein removing the protease comprises incubating the bone graft and protease in a salt solution.

55. The method of claim 54, wherein the salt solution is a NaCl solution.

56. The method of claim 55, wherein the salt solution contains between about 0.15 M NaCl and about 1.5 M NaCl.

57. The method of claim 56, wherein the salt solution contains about 0.3 M NaCl.

58. A method for decreasing the protease activity associated with a bone graft comprising removing at least a portion of the total amount of a protease associated with the bone graft from the bone graft by incubating the bone graft in a salt solution containing about 0.15 M NaCl to about 1.5 M NaCl.

59. A method for treating an individual, the method comprising administering a bone graft and a polypeptide of interest to an individual, wherein the bone graft has been selected based on the level of protease activity.

60. The method of claim 59, wherein at least a portion of the total amount of a protease associated with the bone graft has been removed from the bone graft prior to administering the bone graft to the individual.

61. The method of claim 59, wherein the protease activity of two or more bone grafts is measured, and the bone graft with the lowest protease activity is administered to the individual in conjunction with the polypeptide of interest.

62. The method of claim 59, wherein the protease activity of the selected bone graft is less than about 50 trypsin equivalents.

63. The method of claim 59, wherein selecting the bone graft with an acceptable level of protease activity comprises:
(i) removing at least a portion of the total amount of a protease associated with the bone graft from the bone graft; and
(ii) measuring the amount of a polypeptide substrate that is cleaved by the removed protease, thereby determining the amount of protease activity associated with the bone graft.

64. The method of claim 63, wherein step (i) comprises increasing the ionic strength of the solution comprising the bone graft and protease.

65. The method of claim 64, wherein step (i) comprises incubating the bone graft and protease in a salt solution.

66. The method of claim 65, wherein the salt solution is a NaCl solution.

67. The method of claim 66, wherein the salt solution contains between about 0.15 M NaCl and about 1.5 M NaCl.

68. The method of claim 67, wherein the salt solution contains about 0.3 M NaCl.

69. The method of claim 63, wherein step (ii) comprises separating the cleaved polypeptide substrate, the uncleaved polypeptide substrate, and the bone graft.

70. The method of claim 69, wherein high-performance liquid chromatography is used to separate the cleaved polypeptide substrate, the uncleaved polypeptide substrate, and the bone graft.

71. The method of claim 69, wherein size exclusion chromatography is used to separate the cleaved polypeptide substrate, the uncleaved polypeptide substrate, and the bone graft.

72. The method of claim 59, wherein selecting the bone graft with an acceptable level of protease activity comprises measuring the amount of the polypeptide substrate that is cleaved by a protease activity associated with the bone graft.

73. The method of claim 72, wherein measuring the amount of cleaved polypeptide substrate comprises:
(i) incubating a polypeptide substrate with the bone graft,
(ii) removing at least a portion of the total amount of cleaved polypeptide substrate from the bone graft, and
(iii) measuring the amount of cleaved polypeptide substrate.

74. The method of claim 73, wherein step (ii) comprises increasing the ionic strength of the solution comprising the bone graft and the polypeptide substrate.

75. The method of claim 74, wherein step (ii) comprises incubating the bone graft and the polypeptide substrate in a salt solution.

76. The method of claim 75, wherein the salt solution is a NaCl solution.

77. The method of claim 76, wherein the salt solution contains between about 0.15 M and about 2.0 M NaCl.

78. The method of claim 77, wherein the salt solution contains about 0.6 M NaCl.

79. The method of claim 73, wherein step (iii) comprises separating the cleaved polypeptide substrate, the uncleaved polypeptide substrate, and the bone graft.

80. The method of claim 79, wherein high-performance liquid chromatography is used to separate the cleaved polypeptide substrate, the uncleaved polypeptide substrate, and the bone graft.

81. The method of claim 79, wherein size exclusion chromatography is used to separate the cleaved polypeptide substrate, the uncleaved polypeptide substrate, and the bone graft.

82. The method of claim 63 or 73, wherein the polypeptide of interest and the polypeptide substrate are the same.

83. The method of claim 63 or 73, wherein the polypeptide of interest and the polypeptide substrate are different.

84. The method of claim 59, wherein the polypeptide of interest is PDGF.

85. A method for treating an individual, the method comprising administering a bone graft and PDGF to an individual, wherein the protease activity of the bone graft is less than about 50 trypsin equivalents.

86. A method for treating an individual, the method comprising administering a bone graft and a polypeptide of interest to an individual, wherein at least a portion of the total amount of a protease associated with the bone graft has been removed from the bone graft.

87. The method of claim 86, further comprising measuring the amount of protease that remains associated with the bone graft.

88. The method of claim 86, wherein removing at least a portion of the total amount of a protease associated with the bone graft comprises increasing the ionic strength of the solution comprising the bone graft and protease.

89. The method of claim 88, wherein removing at least a portion of the total amount of a protease associated with the bone graft comprises incubating the bone graft and protease in a salt solution.

90. The method of claim 89, wherein the salt solution is a NaCl solution.

91. The method of claim 90, wherein the salt solution contains between about 0.15 M NaCl and about 1.5 M NaCl.

92. The method of claim 91, wherein the salt solution contains about 0.3 M NaCl.

93. The method of claim 86, wherein the polypeptide of interest is PDGF.

94. A method for treating an individual, the method comprising administering a bone graft and PDGF to an individual, wherein at least a portion of the total amount of a protease associated with the bone graft has been removed from the bone graft by incubating the bone graft in a salt solution.


SEQUENCE LISTING (1 sequence)

SEQ ID NO: 1 - protein, 108 aa, Artificial Sequence
  Synthetic Construc
SLGSLTIAEPAMIAECKTRTEVFEISRRLIDRTNANFLVWPPCVEVQRCSGCCNNRNVQCRPTQVQLRPVQVRKIEIVRKKPIKKATVTLEDHLACKCETVAAARPVT